SEQUENCE LISTING (19 sequences)

SEQ ID NO: 1 - protein, 9 aa, Bos taurus
DTHKSKEIA

SEQ ID NO: 2 - protein, 17 aa, Bos taurus
  misc_feature (1)..(1): Xaa can be any naturally occurring amino acid
XIXNEEQEYIKTVKXSK

SEQ ID NO: 3 - protein, 21 aa, Bos taurus
  misc_feature (1)..(1): Xaa can be any naturally occurring amino acid
XQTPLEKALNSIIDVYHKLAL

SEQ ID NO: 4 - protein, 16 aa, Bos taurus
  misc_feature (14)..(14): Xaa can be any naturally occurring amino acid
MLTAEEKAAVTAFXGK

SEQ ID NO: 5 - protein, 21 aa, Oryctolagus cuniculus
MPTDLENSLNSIIFVYHKCSL

SEQ ID NO: 6 - protein, 21 aa, Mus musculus
MPSELEKALSNITDVYHNYSN

SEQ ID NO: 7 - protein, 21 aa, Homo sapiens
MLTELEKALNSIIDVYHKYSL

SEQ ID NO: 8 - protein, 21 aa, Bos taurus
  misc_feature (1)..(1): Xaa can be any naturally occurring amino acid
XQTPLEKALNSIIDVYHKLAL

SEQ ID NO: 9 - protein, 29 aa, Oryctolagus cuniculus
MAMVSEFLKQAWFIDNEEQDYINTVKTYK

SEQ ID NO: 10 - protein, 29 aa, Mus musculus
MAMVSEELKQARFLENQEQEYVQAVKSYK

SEQ ID NO: 11 - protein, 29 aa, Homo sapiens
MAMVSEFLKQAWFIENEEQEYVQTVKSSK

SEQ ID NO: 12 - protein, 29 aa, Bos taurus
MAMVSEFLKQAWFIENEEQEYIKTVKGSK

SEQ ID NO: 13 - protein, 17 aa, Bos taurus
  misc_feature (1)..(1): Xaa can be any naturally occurring amino acid
XIXNEEQEYIKTVKXSK

SEQ ID NO: 14 - DNA, 33 bp, Artificial Sequence
  Synthetic
gtcgataacatatggcaatggtatcagaattcc

SEQ ID NO: 15 - DNA, 37 bp, Artificial Sequence
  Synthetic
aacgtacttcatatgttagtttcctccacaaagagcc

SEQ ID NO: 16 - DNA, 15 bp, Artificial Sequence
  Synthetic
gatgctgatgaactt

SEQ ID NO: 17 - DNA, 15 bp, Artificial Sequence
  Synthetic
aagttcatcagcatc

SEQ ID NO: 18 - DNA, 12 bp, Artificial Sequence
  Synthetic
ccacaacttcgc

SEQ ID NO: 19 - DNA, 12 bp, Artificial Sequence
  Synthetic
gcgaagttgtgg

(19) United States
(12) Patent Application Publication
(10) Pub. No.: US 2010/0249383 A1
(43) Pub. Date: Sep. 30, 2010

(54) Novel Ligand Involved In The Transmigration Of Leukocytes Across the Endothelium and Uses Therefor

(75) Inventors: Hudson Freeze, San Diego, CA (US); Geetha Srikrishna, La Jolla, CA (US); Ajit Varki, Del Mar, CA (US); Nissi Varki, Del Mar, CA (US)

Correspondence Address: MEDLEN & CARROLL, LLP, 101 HOWARD STREET, SUITE 350, SAN FRANCISCO, CA, 94105, US

(73) Assignee: The Regents of The University of California and The Burnham Institute

(21) Appl. No.: 12/501,926

(22) Filed: Jul. 13, 2009

Related U.S. Application Data
(62) Division of application No. 10/500,480, filed on Dec. 13, 2004.
(60) Provisional application No. 60/346,405, filed on Dec. 28, 2001.

(51) Int. Cl. C07K 16/00; C12N 5/12

(52) U.S. Cl. 530/388.1; 435/326

(57) ABSTRACT

The present invention provides methods and compositions involved in the inflammatory response, as well as various pathological processes. In particular, the present invention provides novel antibodies directed against novel glycans that are enriched on endothelial cell surfaces. In addition, the present invention provides methods and compositions involved in a previously unrecognized pathway of the inflammatory response and various pathological processes. In addition the present invention provides methods and compositions suitable to mediate the inflammatory response in various settings, as well as methods and compositions for the identification of other inflammatory response mediators.

The present invention was made with government support from the U.S. Public Health Service Grant Nos. P01-CA71932, CA38701, and RO1-NS32717. The United States Government has certain rights in the invention.

### FIELD OF THE INVENTION

The present invention provides methods and compositions involved in the inflammatory response, as well as various pathological processes. In particular, the present invention provides novel antibodies directed against novel glycans that are enriched on endothelial cell surfaces. In addition, the present invention provides methods and compositions involved in a previously unrecognized pathway of the inflammatory response and various pathological processes. In addition, the present invention provides methods and compositions suitable to mediate the inflammatory response in various settings, as well as methods and compositions for the identification of other inflammatory response mediators.

### BACKGROUND OF THE INVENTION

Inflammation, a tissue reaction to injury, comprises a complex series of vascular, humoral, and cellular events at or near the site of injury. Inflammation was described in the first century A.D., by Aurelius Celsus. He described inflamed tissues as being hot, painful, swollen, and red. In the following century, Galen added loss of function to the list of cardinal symptoms of inflammation. Inflammation is commonly caused by microorganisms, physical trauma, thermal injury, radiation, foreign bodies, and immune reactions. Because of the numerous biologically active substances involved, the inflammatory responses caused by microorganisms are complex and variable. Thus, most experimental investigations are conducted using physical or chemical injuries, as the response to these types of injury is less complex.

There are three general classes of inflammation, namely acute, chronic, and granulomatous. Acute inflammation is a short-lived (hours or days) process that develops in response to a single injury episode. Chronic inflammation is prolonged (lasting weeks or years), in response to continuous or multiple injuries. Granulomatous inflammation is a particular type of chronic inflammation that develops in response to certain specific agents (e.g., mycobacteria and many fungi).

The vascular events typically observed in inflammation include transient vasoconstriction, arteriolar dilatation, capillary and venule dilatation, and increased vascular permeability. Vasodilation is associated with an increase in the permeability of blood vessels. During this process, there is a microscopic separation of the vascular endothelial cells and the interendothelial cell space becomes sufficiently wide to allow passage of large protein molecules, including fibrinogen and immunoglobulins. Usually, the cellular events begin after the vascular phase has continued for several hours. The leukocytes typically involved in the cellular events in inflammation include neutrophils, macrophages and monocytes, eosinophils, basophils, and lymphocytes. The cellular events usually follow the sequence of margination, pavementing, emigration, aggregation, and phagocytosis. In margination, the leukocytes in the blood vessels (and occasionally, erythrocytes and platelets) move to the periphery of the vessels, allowing the cells to adhere to the endothelial surface (i.e., pavementing). Thus, during inflammation, the normally mutually repellant action of leukocytes and endothelial cells is lost. Although the basic cause remains unknown, the phenomenon can be almost completely inhibited by corticosteroids, indicating that the leukocyte and/or endothelial cell membranes are altered during the inflammatory response (See e.g., Cawson et al., Pathologic Mechanisms and Human Disease, C. V. Mosby Co., St. Louis, Mo. [1982], pages 46-65).

Once they have adhered to the endothelium, the leukocytes begin to emigrate through the vessel walls into the perivascular tissues. Neutrophils are the most active cells, followed closely by monocytes, and then by lymphocytes. In some cases, erythrocytes also escape from the blood vessel, resulting in tissue hemorrhaging. After emigration, the leukocytes are attracted to the site of injury and use chemotaxis (i.e., leukotaxis) to reach the site. The mechanisms involved in leukotaxis largely remain unknown, although recruitment of leukocytes into sites of inflammation depends on a cascade of molecular events, many of which have been delineated in the last decade. The cloning of various factors, as well as the definition of the roles played by selectins, integrins and the endothelial adhesion molecules have contributed to the vast literature on what is currently known regarding leukocyte emigration (See e.g., Springer, in Paul and Issekutz (eds.), Adhesion Molecules in Health and Disease, Marcel Dekker, Inc., NY, page 1 [1997], for a review). However, little is known about molecules involved in transmigration across the endothelium, and subsequent processes in the migration of leukocytes. How the system down-regulates extravasation, and what leads to the cascade of events being perpetuated in chronic inflammation are also less well established.

Typically, acute bacterial infections stimulate the greatest aggregation of leukocytes at or near the site of injury. Initially, the cells are primarily neutrophils, although monocytes and macrophages become increasingly common as time passes. Lymphocytes are usually rare in sites of acute inflammation except in some viral and bacterial infections. At the injury site, phagocytic cells (e.g., neutrophils and macrophages) are active in removing tissue debris, microorganisms, damaged cells, etc.

Various humoral substances (i.e., chemical mediators of inflammation) are also involved in the stimulation and control of the various aspects of inflammation. These substances cause vasodilation, increased vascular permeability, contraction of smooth muscle, attraction of leukocytes (leukotaxis), and production of pain. Vasoactive compounds are particularly important mediators of inflammation, including histamine, serotonin, proteolytic enzymes, and various peptides. Mediators of leukotaxis include such agents as complement complexes (C5, C6, and C7), fibrin split products, kallikrein, and chemotactic substances produced by microorganisms. Although much knowledge has been gained regarding inflammation, much remains unknown, particularly in the area of the chemical mediators responsible for various stages of inflammation. In addition, much remains to be determined in the area of alleviating inflammation and treating inflammatory processes associated with disease. Thus, what is needed are compositions and methods for assessing the various aspects of inflammation, as well as means to alleviate and/or mediate the inflammatory response.

### SUMMARY OF THE INVENTION

The invention provides compositions and methods for diagnosing, and ameliorating diseases such as inflammation and cancer. In particular, the invention provides a method for purifying a carboxylated glycan, the method comprising: a) providing: i) a molecule comprising a carboxylated glycan; ii) biotinylated diamino pyridine (BAP); and iii) an exoglycosidase; b) conjugating the molecule to the BAP to produce a BAP-glycan conjugate; c) treating the BAP-glycan conjugate with the exoglycosidase to produce a first treated BAP-glycan conjugate comprising a first anionic BAP-glycan conjugate having from 1 to 2 negative charges per molecule; and d) isolating the first anionic BAP-glycan conjugate, thereby purifying a carboxylated glycan. In one embodiment, the method further comprises the steps of e) treating the first anionic BAP-glycan conjugate produced in step c) or step d) with an exoglycosidase to produce a second anionic treated BAP-glycan conjugate comprising a second anionic BAP-glycan conjugate having from 1 to 2 negative charges per molecule; and f) isolating the second anionic BAP-glycan conjugate, thereby purifying a carboxylated glycan. In a more preferred embodiment, the method further comprises repeating steps e) and f) from 1 to 10 times. In an alternative embodiment, the molecule is a glycoprotein or polysaccharide. In another alternative embodiment, the step of isolating comprises fractionating by ion exchange chromatography.

The invention also provides a method for purifying a carboxylated glycan, the method comprising: a) providing a molecule comprising a carboxylated glycan; b) isolating from the molecule a first anionic glycan containing from 1 to 4 negative charges; and c) desialylating the isolated first anionic glycan to produce a desialylated anionic glycan containing from 1 to 4 negative charges, thereby purifying a carboxylated glycan. In one embodiment, the method further comprises d) isolating from the first desialylated anionic glycan a second anionic glycan containing from 1 to 4 negative charges, thereby purifying a carboxylated glycan. In an alternative embodiment, the method further comprises prior to step a) the step of treating the molecule with a proteinase enzyme.

The invention additionally provides a method for identifying a test agent as reducing specific binding of a polypeptide to a carboxylated glycan, comprising: a) providing: i) a carboxylated glycan purified by the any one or more of the above-described methods; ii) an antibody that specifically binds to the carboxylated glycan; and iii) a test agent; b) contacting the purified carboxylated glycan, the antibody, and the test agent; and c) detecting a reduction in the level of binding of the antibody to the carboxylated glycan in the presence of the test agent compared to in the absence of the test agent, thereby identifying the test agent as reducing specific binding of a polypeptide to a carboxylated glycan. In one embodiment, the method further comprises d) identifying the test agent as reducing inflammation or cancer. In an alternative embodiment, the purified carboxylated glycan is attached to a solid surface. In a more preferred embodiment, the carboxylated glycan attached to the solid surface is purified by one or more of the above-described methods. In another alternative embodiment, the molecule comprising the carboxylated glycan is a glycoprotein or polysaccharide. In a more preferred embodiment, the molecule is a glycoprotein. In a yet more preferred embodiment, the glycoprotein is a receptor for advanced glycation end products (RAGE). In a further preferred embodiment, the antibody is monoclonal, preferably, the monoclonal antibody is an IgG antibody. In an alternative embodiment, the antibody is specific for a carboxylated glycan purified by the one or more of the methods described above. In a more preferred embodiment, the antibody is monoclonal, preferably the monoclonal antibody is an IgG antibody, more preferably the monoclonal IgG antibody is mAbEE4.1, mAbGB3.1, mAbB2.6, or mAbEH2.7, and yet more preferably the monoclonal IgG antibody is mAbGB3.1.

Also provided herein is a method for identifying a test agent as reducing specific binding of a polypeptide to a carboxylated glycan, comprising: a) providing: i) a carboxylated glycan purified by any one or more of the above methods; ii) leukocyte cells; and iii) a test agent; b) contacting the purified carboxylated glycan, the leukocyte cells, and the test agent; and c) detecting a reduction in the level of adhesion of the leukocytes to the purified carboxylated glycan in the presence of the test agent compared to in the absence of the test agent, thereby identifying the test agent as reducing specific binding of a polypeptide to a carboxylated glycan. In a preferred embodiment, the method further comprises d) identifying the test agent as reducing inflammation or cancer. Preferably, the carboxylated glycan is attached to a solid surface. In another embodiment, the molecule comprising the carboxylated glycan is isolated from endothelial cells. Alternatively, the molecule comprising the carboxylated glycan is a glycoprotein or polysaccharide. Preferably, the glycoprotein is a receptor for advanced glycation end products (RAGE).

Also provided by the instant invention is a carboxylated glycan purified by a method comprising: a) providing: i) a molecule comprising a carboxylated glycan; ii) biotinylated diamino pyridine (BAP); and an exoglycosidase; b) conjugating the molecule to the BAP to produce a BAP-glycan conjugate; c) treating the BAP-glycan conjugate with the exoglycosidase to produce a first treated BAP-glycan conjugate comprising a first anionic BAP-glycan conjugate having from 1 to 2 negative charges per molecule; and d) isolating the first anionic BAP-glycan conjugate, thereby purifying a carboxylated glycan. In one embodiment, the molecule comprising the carboxylated glycan is a glycoprotein or polysaccharide. More preferably, the glycoprotein is a receptor for advanced glycation end products (RAGE).

The invention additionally provides carboxylated glycan purified by a method comprising: a) providing a molecule comprising a carboxylated glycan; b) isolating from the molecule a first anionic glycan containing from 1 to 4 negative charges; and c) desialylating the isolated first anionic glycan to produce a desialylated anionic glycan containing from 1 to 4 negative charges, thereby purifying a carboxylated glycan. In one embodiment, the molecule comprising the carboxylated glycan is a glycoprotein or polysaccharide. More preferably, the glycoprotein is a receptor for advanced glycation end products (RAGE).

Also provided herein is an antibody produced by EE4.1 cells, GB3.1 cells, B2.6 cells, or EH2.7 cells. Preferably, the antibody is produced by GB3.1 cells.

The invention additionally provides an antibody specific for a carboxylated glycan purified by any one or more of the above described methods. In one embodiment, the binding of the antibody to the carboxylated glycan is reduced by a carboxylated glycan, and the binding is not reduced by a carboxylate-neutralized glycan selected from an alkyl esterified glycan or alkylamidated glycan. Preferably, the alkyl esterified glycan is CONH-methyl-glycan and the alkylamidated glycan is a methylamidated glycan. In an alternative embodiment, the antibody is monoclonal, preferably the monoclonal antibody is an IgG antibody, more preferably the monoclonal IgG antibody is mAbGB3.1 antibody. In an alternative embodiment, the antibody does not specifically bind to glucuronic acid, galacturonic acid, sialic acid, lactic acid, pyruvic acid, or uronic acid. Alternatively, the antibody does not specifically bind to a sulfated glycan, wherein the sulfated glycan is contained in thyroglobulin or neural cell adhesion molecule (N-CAM). In yet another alternative, the antibody does not specifically bind to a glycosaminoglycan, such as a glycosaminoglycan contained in chondrosamine, chondroitin sulfate, chondroitin sulfate tetramer, chorndroitin sulfate octamer, hyaluronic acid tetramer, hyaluronic acid octamer, heparin, or heparan sulfate. In a further alternative, the antibody does not specifically bind to a phosphorylated sugar selected from the group consisting of glucose-1-phosphate, glucose-6-phosphate, mannose-6-phosphate, galactose-6-phosphate, glucose-N-acetyl-1-phosphate, and glucose-N-acetyl-6-phosphate. In yet another alternative, the antibody does not specifically bind to a sulfated sugar selected from the group consisting of glucose-6-sulfate and galactose-6-sulfate. In a further alternative, the molecule is a glycoprotein or polysaccharide, preferably the glycoprotein is a receptor for advanced glycation end products (RAGE), more preferably the antibody is monoclonal, yet more preferably the monoclonal antibody is an IgG antibody.

Also provided by the invention is a hybridoma cell line that produces a monoclonal antibody selected from the group consisting of mAbEE4.1, mAbGB3.1, mAbB2.6, and mAbEH2.7, more preferably the hybridoma cell line produces monoclonal antibody mAbGB3.1.

The invention additionally provides a method for reducing extravasation of leukocyte cells in endothelial tissue, comprising: a) providing: i) endothelial tissue comprising leukocyte cells; and ii) an agent that reduces specific binding of a polypeptide to a carboxylated glycan purified by any one or more of the above described methods; and b) administering the agent to the endothelial tissue such that specific binding of the polypeptide to the carboxylated glycan is reduced, thereby reducing extravasation of the leukocyte cells in the endothelial tissue. In one embodiment, the molecule comprising the carboxylated glycan is a glycoprotein or polysaccharide, preferably the glycoprotein is a receptor for advanced glycation end products (RAGE). Alternatively, the polypeptide comprises S100A8, S100A9, S10012, amphoterin, annexin I, or amino acids 1 to 12 of annexin I. More preferably, the polypeptide comprises a S100A8-S100A9 heterodimer, (S100A8)₂·S100A9 heterotrimer, or (S100A8)₂·(S100A9)₂ heterotetramer. In one alternative, the polypeptide comprises S100A12. In yet another alternative, the polypeptide comprises amino acids 1 to 12 of annexin I, preferably, the polypeptide comprises amino acids 1 to 40 of annexin I, more preferably, the polypeptide comprises annexin I. In another alternative, the polypeptide comprises amphoterin. In one embodiment, the agent is identified by any one or more of the above described methods. In another embodiment, the agent is an antibody specific for the carboxylated glycan, preferably, the antibody is monoclonal, more preferably, the monoclonal antibody is an IgG antibody, yet more preferably, the monoclonal IgG antibody is mAbEE4.1, mAbGB3.1, mAbB2.6, or mAbEH2.7. In another embodiment, the agent is an anti-S100A8 antibody, anti-S100A9 antibody, anti-S100A12 antibody, anti-annexin I antibody, an antibody specific for amino acids 1 to 12 of annexin I, an antibody specific for amino acids 1 to 40 of annexin I, or an anti-amphoterin antibody.

Also provided herein is a method for reducing adherence of leukocyte cells to endothelial cells, comprising: a) providing: i) leukocyte cells; ii) endothelial cells; and iii) an agent that reduces specific binding of a polypeptide to a carboxylated glycan purified by any one or more of the above described methods; and b) contacting the leukocyte cells, the endothelial cells, and the agent such that adherence of the leukocyte cells to the endothelial cells is reduced in the presence of the agent compared to in the absence of the agent. In one embodiment, the polypeptide comprises S100A8, S100A9, S10012, amphoterin, annexin I, or amino acids 1 to 12 of annexin I. In another embodiment, the agent is identified by any one or more of the above described methods. In an alternative embodiment, the agent is an antibody specific for the carboxylated glycan. Preferably, the antibody is monoclonal, more preferably, the monoclonal antibody is an IgG antibody, yet more preferably, the monoclonal IgG antibody is mAbEE4.1, mAbGB3.1, mAbB2.6, or mAbEH2.7.

The invention also provides a method for reducing inflammation in a tissue in a mammalian subject, comprising: a) providing: i) a tissue; and an agent that reduces specific binding of a polypeptide to a carboxylated glycan purified by any one or more of the above-described methods; and b) administering the agent to the tissue such that inflammation in the tissue is reduced in the presence of the agent compared to in the absence of the agent. In one embodiment, the mammalian subject is human. In another embodiment, the agent is identified by any one or more of the above-described methods. In one alternative, the antibody is monoclonal, preferably an IgG antibody, more preferably is mAbEE4.1, mAbGB3.1, mAbB2.6, or mAbEH2.7. In another alternative, the antibody is conjugated to a cytotoxin. Alternatively, the antibody is conjugated to an imaging molecule. In a further alternative, the antibody is chimeric. In one embodiment, the administering is before, concomitant with, and/or after manifestation of inflammation in the tissue. In a further embodiment, the human subject has or is suspected of being capable of developing Crohn's disease, tumor growth, metastasis, diabetes, Alzheimer's disease, dementia, atherogenesis, periodontal disease, skin immune responses, septic shock, heart disease, arthritis, sarcoidosis, tuberculosis, chronic inflammation, acute inflammation, endotoxic shock, ulcerative colitis, multiple sclerosis, anaphylactic reaction, nephritis, asthma, conjunctivitis, systemic lupus erythematosus, ocular inflammation, allergy, emphysema, ischemia-reperfusion injury, fibromyalagia, psoriasis, rheumatoid arthritis, gouty arthritis, juvenile rheumatoid arthritis, and osteoarthritis. In a particular embodiment, the human subject has or is suspected of being capable of developing Crohn's disease or ulcerative colitis. In one alternative embodiment, the polypeptide comprises S100A8, S100A9, S 10012, amphoterin, annexin I, or amino acids 1 to 12 of annexin I.

Also provided by the instant invention is a method for reducing cancer in a mammalian subject, comprising: a) providing: i) a mammalian subject; and ii) an agent that reduces specific binding of a polypeptide to a carboxylated glycan purified by any one or more one of the above described methods; and b) administering the agent to the subject such that cancer in the subject is reduced in the presence of the agent compared to in the absence of the agent. In one embodiment, the mammalian subject is human. In one embodiment, the agent is an antibody specific for the carboxylated glycan. In another embodiment, antibody is monoclonal, preferably an IgG antibody, more preferably is mAbEE4.1, mAbGB3.1, mAbB2.6, or mAbEH2.7. In one embodiment, the antibody is conjugated to a cytotoxin. In another embodiment, the antibody is conjugated to an imaging molecule. In an alternative embodiment, the antibody is chimeric. In a further embodiment, the administering is before, concomitant with, and/or after manifestation of cancer. In further embodiment, the polypeptide comprises S100A8, S100A9, S10012, amphoterin, annexin I, or amino acids 1 to 12 of annexin I.

### DESCRIPTION OF THE DRAWINGS

FIG. 1 provides HPLC results for BAP-coupled anionic bovine lung glycans of moderate negative charge that were treated with multiple exoglycosidases. Panel A provides the results for a sample refractionated on a DEAE-2SW HPLC column. In this Panel, arrowheads indicate the elution positions of standard neutral glycans (0), glycans containing one sulfate (S-1), and glycans containing two sulfates (S-2). Panel B provides the results for aliquots of pools I, II, and III that were collected and analyzed by reverse phase HPLC.

FIG. 2 provides characterizations of mAbGB3.1 reactivity. Panel A shows results for bovine lung proteins probed with mAbGB3.1 hybridoma supernatant (20 ng IgG/ml), in the presence or absence of varying concentrations of bovine lung asialo-COO⁻ and asialo-CONHMe-glycopeptides. Binding in the absence of inhibitor was defined as 100%. Panel B provides results for bovine lung proteins and Dictyostelium discoideum lysates probed with mAbGB3.1, AD7.5 or HF3.3, at different pH values as indicated. Antibody AD7.5, recognizes GlcNAc-1-P residues on Dictyostelium cysteine proteinases (Mehta et al., J. Biol. Chem., 271:10897 [1996]), while HF3.3 is another antibody from the group of hybridomas isolated during the development of the present invention. Its reactivity with bovine lung proteins is unaffected by carboxylate-neutralization. Binding at pH 7.0 was defined as 100% for each. Panel C provides results for bovine lung proteins probed with mAbGB3.1 hybridoma supernatant (20 ng IgG/ml), in the presence or absence of varying concentrations of seven different carboxylate containing compounds. Binding in the absence of inhibitor was defined as 100%.

FIG. 3 provides data that indicate a major fraction of the mAbGB3.1 reactive-epitope is carried on N-linked glycans, including PNGase F resistant ones. Reactivity in control wells was defined as 100%.

FIG. 4 provides Western blot results for homogenized human tissues and 50 μg protein from various extracts probed with mAbGB3.1 (400 ng/ml). The upper portion of this Figure shows results for antibody incubation conducted in the absence of total bovine lung acetone powder glycopeptides, while the lower panel shows the results for antibody incubation conducted in the presence of total bovine lung acetone powder glycopeptides. The blots were then developed with anti-mouse IgG-alkaline phosphatase conjugates and BCIP-NBT substrate.

FIG. 5 provides photomicrographs of frozen sections of various human tissues that were immunostained with mAbGB3.1. The reddish-brown reaction product indicates prominent staining on vascular endothelium. Panel A shows the results with placental tissue, while Panel B provides results for kidney tissues, Panel C provides results for lung tissue, Panel D provides results for heart tissue, Panel E, provides results for spleen tissue, and Panel F provides results for liver tissue.

FIG. 6 shows the expression of GB3.1 reactive epitopes on cultured endothelial cells. Panel A provides the results for CAB4 (negative control) antibody, while Panel B provides the results for purified mAbGB3.1 IgG.

FIG. 7 shows the results of FACS analysis of neutrophils for carboxylated glycan-binding sites. Panel A provides results for resting cells, while Panel B provides results for stimulated cells.

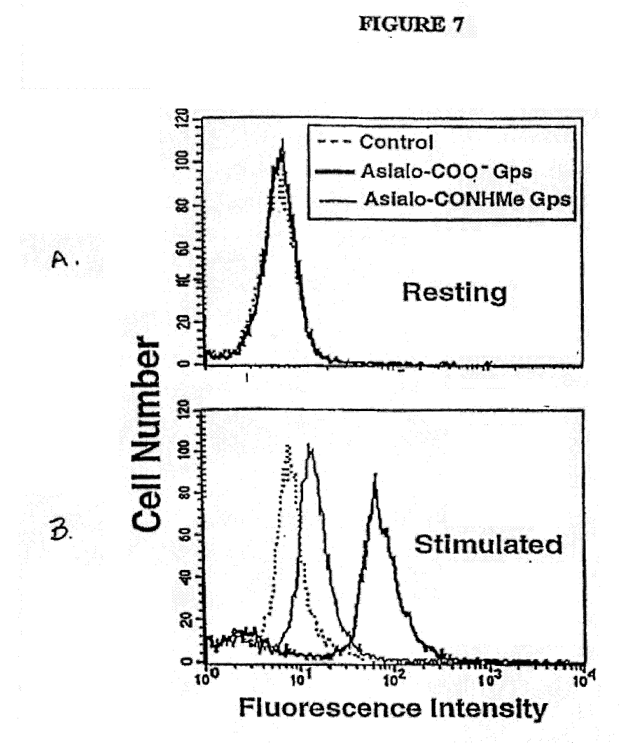

FIG. 8 provides graphs showing the specific adhesion of neutrophils to carboxylated glycans coated on plastic. Panel A provides the binding response in the presence of antibodies, while Panel B provides the binding response in the absence of antibodies. The data are presented as the mean±SD of duplicate assays.

FIG. 9 provides graphs showing the effect of mAbGB3.1 on neutrophil (Panel A) and monocyte (Panel B) accumulation in the inflamed mouse peritoneum. The numbers indicate μg of antibody injected per gram of mouse body weight. The data are presented as the mean±SD of 4 mice per group.

FIG. 10 provides photomicrographs showing the effect of mAbGB3.1 on leukocyte sequestration in mesenteric vasculature during peritoneal inflammation. The upper panels (Panel A) taken at 250× magnification show extravascular infiltration of leukocytes (seen as dark dots) in inflamed tissues, which is reduced by mAbGB3.1 treatment, but unaltered by treatment with a control monoclonal antibody. The lower panels (Panel B) at higher magnification (2500×) of representative veins focus on the accumulation of leukocytes along inflamed vessels in saline and control antibody treated mice, and marked increased numbers of adherent leukocytes along the venular endothelium in mAbGB3.1 treated mice.

FIG. 11 is a graph showing the inhibition of mAbGB3.1 binding to BSA-conjugated bovine lung glycopeptides by various DEAE fractions of bovine lung glycopeptides.

FIG. 12 provides silver-stained SDS-PAGE gels of proteins eluted from bovine lung glycopeptide affinity columns. Panel A contains eluates from a column containing immobilized 0.1 asialo-COO⁻ bovine lung glycopeptides (lane 1); a corresponding control column containing immobilized 0.1 M asialo-CONHMe-glycopeptides (lane 2); a column containing immobilized 0.3 M asialo-COO⁻ bovine lung glycopeptides (lane 3); and a corresponding control column containing 0.3 M asialo-CONHMe-glycopeptides. Panel B provides the results for eluted proteins from lanes 1 and 3 that had been pooled, dialyzed and reloaded onto the column containing immobilized 0.1 M asialo-COO⁻ lung glycopeptides. Unbound fractions are in lane 5, while lane 6 contains proteins eluted by 5 mM citrate in starting buffer. Arrows indicate protein bands that bound and rebound in a carboxylate-dependent manner

FIG. 13 shows results from the N-terminal sequences of bovine lung proteins eluted from the carboxylate-enriched glycopeptide columns. Bovine lung proteins eluted from the glycopeptide affinity column were separated on 12% SDS-PAGE gels and transferred to PVDF membranes. Proteins were visualized using 0.1% Coomassie brilliant blue. Individual bands were excised and N-terminal Edman sequencing was performed on a Model 494 Procise Sequencer. In this Figure, the SDS-PAGE stained bands, N-terminal sequence information, and identity of four protein bands are shown. The 67 kDa protein band was found to have the N-terminal sequence “DTHKSKEIA” (SEQ ID NO:1), and corresponded to albumin. The 37 kDa protein band was found to have the N-terminal sequence “XIXNEEQEYIKTVKXSK” (SEQ ID NO:2), and corresponded to annexin I. The 35 kDa protein band was found to have the N-terminal sequence “XQTPLEKALNSIIDVYBKLAL” (SEQ ID NO:3), and corresponded to S100A8. The <19 kDa protein band was found to have the N-terminal sequence “MLTAEEKAAVTAFXGK” (SEQ ID NO:4), and corresponded to hemoglobin (β-chain).

FIG. 14 provides results showing that human homologues of the S100 proteins and annexin I bind to the novel carboxylated bovine lung glycans. Neutrophil lysates were incubated with BSA-conjugated 0.3 M asialo-COO⁻ glycopeptides immobilized on microtiter plates and the wells were developed with anti-annexin I (Panel A), and anti-S100A8 (Panel B), or anti-S100A9 (not shown). Each point is the mean±S.D. of three determinations. The insets within this Figure provide data for neutrophil lysates loaded onto carboxylated (lanes 1 and 2) or neutralized (lanes 3 and 4) glycopeptide affinity columns and eluted with citrate. Unbound and bound fractions were analyzed by Western blotting using anti-annexin I (Panel A) or anti-S 100A8 (Panel B) antibodies. Lanes 1 and 3 represent unbound fractions, while lanes 2 and 4 represent bound fractions. The identity of the S100A8/A9 heterodimer (marked by an asterisk) in the glycopeptide bound fraction (Panel B, lane 2) was confirmed by a separate immunoblotting with anti-S100A9 (data not shown).

FIG. 15 shows results that indicate human S100A8 binds to the carboxylated glycans as a heteromeric complex with S100A9, and an intact N-terminus may be necessary for optimal human annexin I binding. In Panel A, lane 1 contains a sample assayed at 0 time; lane 2 contains a sample assayed after binding to the glycopeptides for 2 hours; lane 3 contains a sample assayed after binding to the immobilized glycopeptides for 2 hours in the presence of 200 μM 0.3 M asialo-COO⁻ glycopeptides; lane 4 contains a sample assayed after binding to the immobilized glycopeptides for 2 hours in the presence of 200 μM 0.3 M asialo-CONHMe-glycopeptides. Panel B shows the Western blot results for supernatants tested with anti-annexin I. Lane 1 contains a sample assayed at 0 time; lane 2 contains a sample assayed after binding to the glycopeptides for 2 hours; lane 3 contains a sample assayed after binding to the glycopeptides for 2 hours in the presence of 200 μM 0.3 M asialo-COO⁻ glycopeptides.

FIG. 16 provides results showing that an intact N-terminus may be necessary for human annexin I to bind to carboxylated glycans. Freshly activated human neutrophil lysates were incubated with BSA conjugated 0.3 asialo-COO⁻ bovine lung glycopeptides coated on microtiter plates. After incubation, supernatants were collected and analyzed by immunoblots using anti-annexin I, and chemiluminescence detection. Lane 1 shows zero time, while lane 2 provides results after binding to the immobilized glycopeptides for 2 hr, and lane 3 provides results after binding to the immobilized glycopeptides for 2 hr in the presence of 200 μM soluble 0.3 M asialo-COO⁻ glycopeptides.

FIG. 17 shows results that indicate neutrophil adhesion to immobilized carboxylated glycans is blocked by mAbGB3.1 and anti-S100A9. Each point is the mean±SD of two to four determinations.

FIG. 18 provides results showing that annexin I and S100A8 are expressed on the cell surface of freshly isolated neutrophils upon activation. Stimulated or resting neutrophils were incubated with anti-annexin I, anti-S100A8 or an isotype-matched control antibody, followed by FITC-conjugated anti-mouse IgG. Cells stained with secondary antibody only were used as negative controls and provide the background staining in the plot overlays.

FIG. 19 shows results that indicate purified human S100A8/9 and annexin I show specific binding to immobilized bovine lung glycans. Panel A contains purified S100A8/A9 complex and Panel B contains annexin I. Each point is the mean±SD of two determinations.

FIG. 20 shows the cell surface binding of ¹²⁵I-S 100A8/A9 to endothelial cells. Panel A provides results for cells incubated with 4 nM to 80 nM ¹²⁵I-S100A8/A9, while Panel B provides results for cells incubated with 20 nM labeled complex in the absence of modulators, and Panel C provides results for cells incubated with 20 nM labeled complex in the presence of modulators. Each point is the mean±SD of two determinations.

FIG. 21 provides a silver-stained gel (lane 1) and immunoblot (lane 2) showing that amphoterin binds to immobilized carboxylated bovine lung glycans. Bovine lung proteins were loaded on COO⁻ glycopeptide affinity columns and eluted as described herein. Lane 1 contains eluted proteins, while lane 2 provides an immunoblot of the eluates. The 67 kD recognized by anti-amphoterin is a cross-reacting species. The 37 and 35 kDa bands on the silver gels are annexin I and S100A8/A9, respectively.

FIG. 22, Panel A provides a graph showing that carboxylated glycopeptides inhibit mAbGB3.1 binding to immobilized sRAGE, but CONHMe-glycans do not. Each point is the mean±SD of two determinations. Panel B provides and immunoblot showing that bovine lung RAGE carries PNGaseF-sensitive, mAbGB3.1-positive, N-linked oligosaccharides. Bovine lung RAGE was immunoprecipitated using mAbGB3.1. The protein was subjected to PNGase F digestion and the native and digested proteins were immunoblotted with anti-RAGE or mAbGB3.1.

FIG. 23 provides data showing that mAbGB3.1 glycotope is expressed on various tumor cells. Panel A provides data from cultured human tumor cells that were analyzed for surface expression of mAbGB3.1 antigens by FACS. Cells were stained with mAbGB3.1 followed by FITC-conjugated anti-mouse IgG. Cells stained with secondary antibody only were used as negative controls, and provide the background staining in the plot overlays. Panel B provides data showing that mAbGB3.1 and anti-RAGE recognize similar proteins in human tumor cells. Proteins from solubilized membrane fractions were analyzed by Western blotting using mAbGB3.1, or anti-RAGE. Protein bands were detected using anti-mouse Ig peroxidase conjugate and ECL reagents. The tumor cell lines used in these experiments were: T98G, glioblastoma; M21, melanoma; SKNSH, neuroblastoma. Panel C provides data from analysis of [2-³H] mannose labeled N-linked oligosaccharides on RAGE immunoprecipitated from SKNSH cells. Labeled oligosaccharides were released with PNGase F and analyzed on QAE-Sephadex columns before and after desialylation, methyl esterification, and methylesterification followed by regeneration of carboxylates.

FIG. 24 provides data showing that the binding of amphoterin to RAGE is glycan-dependent. Panel A provides saturation binding of ¹²⁵I data for binding of amphoterin to RAGE. Non-specific binding was determined in the presence of 100-fold excess of unlabeled amphoterin. Binding to native RAGE was also measured in the presence of 100 mM COO⁻ or neutral glycans. Non-linear regression transforms of the specific binding data using GraphPad Prism are presented here. The values are mean±SEM of triplicate determinations. Panel B provides a graph showing the effect of various concentrations of COO⁻ or neutral glycans on the binding of 10 nM amphoterin to RAGE. The values are mean±SEM of triplicate determinations and were fitted to a non-linear regression (one-site competitive binding equation using GraphPad Prism). Panel C provides a graph showing the effect of 20 μg/ml mAbGB3.1 or an unrelated anti-carbohydrate monoclonal antibody on the binding of 10 nM amphoterin to RAGE. The values are mean±SEM of triplicate determinations.

FIG. 25 provides results showing that mAbGB3.1 reactivity localizes on the surface and growth cones of embryonic cortical neurons spreading on amphoterin. Panel A shows neurons stained with a non-specific control antibody. Note that mAbGB3.1 immunoreactivity is detected along neurites (arrowheads) and in growth cones (arrows). The surfaces of cell bodies (double arrows) were not always stained. In Panel A, the bar represents 5 mM. Panels B and C provide results for cortical neurons grown on amphoterin and stained with mAbGB3.1. Panel B shows that mAbGB3.1 blocks neurite outgrowth on amphoterin substrate. Cortical neurons were allowed to grow on amphoterin in the absence (Panel D) or presence of 20 mg/ml of a non-specific control IgG (Panel E), or mAbGB3.1 (Panel F). The cells were fixed and stained with a lipophilic dye (DiO). In this Panel, the bar represents 40 mM.

FIG. 26 provides data showing that carboxylated glycopeptides promote outgrowth of mouse embryonic cortical neurons. Cortical neurons were isolated and grown on BSA-conjugated COO⁻ or CONHMe-glycopeptides (20 mg/ml) coated on polylysine matrices. Cells grown on polylysine alone served as controls. Morphometric analysis of neurite lengths was performed on captured images of fixed, Dii stained cultures. Neurite-bearing cells were defined as cells bearing neurites greater than one cell body in diameter. The data are presented as the mean±SD of two replicate analyses (**, P<0.005).

FIG. 27 provides data showing that amphoterin- or glycopeptide-induced neurite outgrowth on RAGE transfected cells. In the top panel: serum-starved N18 neuroblastoma cells were stably transfected either with full-length RAGE (Panels A, B, C, and F), or the cytoplasmic domain deletion mutant of RAGE (Panels D and E). They were grown on amphoterin (20 μg/ml; Panels A, C, D and F) in the absence (A, D) or presence of soluble COO⁻ glycopeptides (COO⁻ Gps; 200 μM; Panel C) or mAbGB3.1 (50 μg/ml; Panel F), or on 200 μM COO⁻ glycan-coated surfaces (Panels B and E). In this Figure, the bar represents 40 μM. In the bottom panel, data are provided for quantitation of cells bearing neurites longer than one diameter of the cell soma. Data are mean±SD of three replicate experiments (***, P<0.0005).

FIG. 28 shows the time-dependent loss of body weight of mice treated with control antibody or with antibody mAbGB3.1.

FIG. 29 shows the histological analysis of the colon from immune-deficient mice treated with a control monoclonal antibody or with mAbGB3.1.

FIG. 30 shows the amino acid sequence of human annexin I (GenBank No. NP_{—}000691).

FIG. 31 shows the amino acid sequence of human annexin I (GenBank No. LUHU).

FIG. 32 shows the amino acid sequence of rat annexin I (GenBank No. LURT1).

FIG. 33 shows the % binding of monoclonal antibodies to immobilized BSA neoglycoproteins in the presence of asialo-COO⁻ glycopeptides (open boxes) in solution, or asialo-CONHMe-glycopeptides (closed boxes). Monoclonal antibodies EE4.1, GB3.1, B2.6, and EH2.7 were raised against the carboxylated glycan, and the control monoclonal antibody HF3.3 was raised against N-acetylglycosamine 1-phosphate linked to serine.

### DEFINITIONS

To facilitate understanding of the invention, a number of terms are defined below.

As used herein, the term “leukocyte” refers to white blood cells, including lymphocytes, neutrophils, eosinophils, basophils, monocytes, and macrophages. In a preferred embodiment, the leukocyte cell is a neutrophil cell.

As used herein, the term “endothelium” refers to the cells that internally line the entire circulatory system of humans and other animals. In humans, these cells are simple squamous epithelial cells. Typically, blood vessels are comprised of the tunica intima, the tunica media, and the tunica adventitia. The tunica intima is comprised of a layer of endothelial cells that line the interior surface (i.e., the lumen) of the vessel. Positioned beneath this layer, there is a subendothelial layer consisting of delicate loose connective tissue that may contain some smooth muscle cells. The tunica media and tunica adventitia are comprised primarily of smooth muscle cells, as well as elastin, collagen, and proteoglycans.

As used herein, the term “inflammation” refers to the tissue response of an organism to an injury.

As used herein, the term “chemical mediator of inflammation” refers to any chemical which is involved in producing, moderating, or terminating the inflammatory response. The term encompasses naturally-occurring, as well as synthetic mediators. The term includes, but is not limited to cytokines and other effector molecules. Indeed, it is intended that the term encompass any molecule or compound that affects the inflammatory response in any aspect or by any mechanism (indeed, an understanding of the mechanism involved is not necessary in order to use the present invention).

As used herein, the term “chemotaxis” refers to the movement of cells in response to a chemical stimulus.

As used herein, the term “leukotaxis” refers to the chemotaxis of leukocytes. In particular, the term refers to the tendency of leukocytes to accumulate in regions of injury and inflammation.

As used herein, the terms “S100A8” (also sometimes referred to as “CP-10” or “MRP8”) and “S100A9” (sometimes referred to as “MRP9”) refer to two calcium-binding proteins that are members of the chemotactic S100 protein family of molecules.

As used herein, the term “RAGE” refers to the “receptor for advanced glycation end products.” RAGE is a physiologically important binding protein for amphoterin (Hori et al., J. Biol. Chem., 270:25752-25761 [1995]; Huttunen et al., J. Biol. Chem., 274:19919-19924 [1999]; and Huttunen et al., J. Biol. Chem., 275:40096-40105 [2000]). It is an immunoglobulin superfamily member consisting of an N-terminal V-type domain and two C-type domains. It also has a single transmembrane spanning domain that anchors the protein, and a cytosolic tail which is essential for signaling (Neeper et al., J. Biol. Chem., 267:14998-15004 [1992]). The extracellular V-type domain is believed to be the principal ligand-binding site that interacts with a diverse group of ligands including advanced glycation end products, β-amyloid peptides, and S100A12, in addition to amphoterin (Kislinger et al., J. Biol. Chem., 274:31740-31749 [1999]; and Schmidt et al., Biochim. Biophys. Acta 1498:99-111 [2000b]). Indeed, RAGE engages a series of structurally unrelated ligands (See e.g., Schmidt et al., Sem. Thrombosis Hemostasis 26:485-493 [2000a]; Schmidt and Stern, Trends Endocrinol. Metab., 11:368-375 [2000]; and Schmidt et al., Biochim. Biophys. Acta 1498:99-111 [2000b], for review).

Full length RAGE has two potential N-linked glycosylation sites; its alternatively spliced and secreted isoform present in human lung and brain has an additional site (Malherbe et al., Brain Res. Mol. Brain Res., 71:157-170 [1999]; and Neeper et al., supra). The two N-linked sites on mature RAGE occur in the principal ligand-binding V-domain (Kislinger et al., supra). Prior to the development of the present invention, there were no previous studies that provide the presence of oligosaccharides on RAGE or on their structure. However, experiments conducted during the development of the present invention show that mature RAGE is glycosylated and that the N-linked glycans have the carboxylated epitope. The abundance of mAbGB3.1 reactivity in bovine lung, endothelial cells, macrophages, tumor and embryonal cells parallels the reported tissue expression of RAGE (Brett et al., Am. J. Pathol., 143:1699-1712 [1993]). The function of the alternatively spliced and secreted isoform of RAGE in human brain and lung with three potential N-glycosylation sites is unknown. Nonetheless, an understanding of the mechanism(s) involved is not necessary in order to use the present invention. Incidentally, the additional N-glycosylation site in the alternatively spliced form is not present on the V-domain of the secreted protein. It has been suggested that the soluble form could act as a physiological antagonist, analogous to the addition of sRAGE in experimental systems, where it competes with cell surface RAGE for amphoterin binding (See, Hori et al., supra; Malherbe et al., .supra; Taguchi et al., supra; and Yan et al., Nat. Med., 6:643-651 [2000]). RAGE also binds to a group of seemingly unrelated ligands including advanced glycation end products, 13-amyloid proteins, and S100A12. The structural basis for the multi-ligand binding properties of RAGE is not understood, but data obtained during the development of the present invention indicate that carboxylated modifications on RAGE and/or other associated glycoproteins could be important modulators. Incidentally, S100A12, another established ligand for RAGE, is most homologous to S100A9, which also binds the carboxylated glycans (Robinson and Hogg, Biochem. Biophys. Res. Commun., 275:865-870 [2000]). Regardless, an understanding of the mechanism(s) is not necessary in order to use the present invention.

As used herein, the term “amphoterin” refers to a protein linked to neuronal development and invasive cell migration (Hori et al., J. Biol. Chem., 270:25752-25761 [1995]; Rauvala et al., Matrix Biol., 19:377-387 [2000]; and Rauvala and Pihlaskari, J.

Biol. Chem., 262:16625-16635 [1987]), differentiation of erythroleukemia cells (Melloni et al., Biochem. Biophys. Res. Commun., 210:82-90 [1995b]), endotoxemia (Abraham et al., J. Immunol., 165:2950-2954 [2000]; and Wang et al., Science 285:248-251 [1999]), and tumor growth and metastasis (Taguchi et al., Nature 405:354-360 [2000]). Amphoterin has a highly dipolar charge distribution, and is a developmentally regulated protein that is abundant in embryonic brain and in transformed cell lines (Parkkinen et al., [1993] infra; Rauvala and Pihlaskari, J. Biol. Chem., 262:16625-16635 [1987]). It has extensive sequence similarity to HMGB1 type DNA binding proteins (Merenmies et al., [1991], infra), but its functional role in the nucleus, if any, remains unclear. Amphoterin is also a cytosolic protein that localizes to growth cones of embryonic neuronal cells and leading edges of tumor cells when extension of cytoplasmic processes are stimulated on appropriate matrices (Merenmies et al., [1991], infra; and Parkkinen et al., [1993], infra). RAGE is a major cellular binding site for amphoterin at the leading edges of invasive cells.

Amphoterin is also a late mediator of endotoxin lethality and acute lung inflammation in mice (Abraham et al., J. Immunol., 165:2950-2954 2000; and Wang et al., Science 285:248-251 [1999]). RAGE-amphoterin interaction is a key checkpoint in tumor growth, invasion and metastasis (Taguchi et al., Nature 405:354-360 [2000]). A ternary complex between amphoterin, plasminogen, and plasminogen activator at the leading edge of neurites and tumor cells activates metalloproteinases (MMP-2 and MMP-9) that degrade extracellular matrix molecules (Rauvala et al., [2000]; and Taguchi et al., [2000], supra).

A “variant” of a protein as used herein, refers to an amino acid sequence that is altered by one or more amino acids. The variant may have “conservative” changes, wherein a substituted amino acid has similar structural or chemical properties, (e.g., replacement of leucine with isoleucine). More rarely, a variant may have “nonconservative” changes (e.g., replacement of a glycine with a tryptophan). Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

The term “biologically active,” as used herein, refers to a protein or other biologically active molecules (e.g., catalytic RNA) having structural, regulatory, or biochemical functions of a naturally occurring molecule. Likewise, “immunologically active” refers to the capability of the natural, recombinant, or synthetic molecule, or any oligopeptide or polynucleotide thereof, to induce a specific immune response in appropriate animals or cells and to bind with specific antibodies.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like. A “biological sample” is a sample that has been obtained from an organism and encompasses any type of material (e.g., including but not limited to body fluids, tissue, bone, bone marrow, etc.).

“In operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the terms “vector” and “vehicle” are used interchangeably in reference to nucleic acid molecules that transfer DNA segment(s) from one cell to another.

“Expression construct,” “expression vector,” and “plasmid” as used herein, refer to one or more recombinant DNA or RNA sequences containing a desired coding sequence operably linked to sequences necessary for the expression of the coding sequence in a cell or host organism (e.g., a mammal). The sequence may be single or double stranded.

“Reporter construct,” “reporter gene,” and “reporter protein” as used herein, refer to DNA or amino acid sequences, as appropriate, that, when expressed in a host cell or organism, may be detected, measured or quantitated.

As used herein, the terms “purified” or “to purify” refers to the removal of one or more (undesired) components from a sample. For example, where recombinant polypeptides are expressed in bacterial host cells, the polypeptides are purified by the removal of host cell proteins thereby increasing the percent of recombinant polypeptides in the sample. For example, a carboxylated glycan is purified by at least a 10%, preferably by at least 30%, more preferably by at least 50%, yet more preferably by at least 75%, and most preferably by at least 90%, reduction in the amount of undesirable proteins, glycoproteins, and/or polysaccharides, such as those present in a nuclear and/or cytoplasmic cell extract. Thus purification of a carboxylated glycan results in an “enrichment,” i.e., an increase in the amount, of carboxylated glycan in the sample. The carboxylated glycan may be purified using methods disclosed herein. The terms “purify” and “purifying” denote carrying out one or more steps to generate a purified molecule.

As used herein, the term “partially purified” refers to the removal of a moderate portion of the contaminants of a sample to the extent that the substance of interest is recognizable by techniques known to those skilled in the art as accounting for a measurable amount of the mixture.

As used herein, the term “substantially purified” refers to molecules, (e.g., nucleic or amino acid sequences) that are removed from their natural environment, isolated or separated, and are at least 60% free, preferably 75% free and more preferably 90% free from other components with which -they are naturally associated. Furthermore, an “isolated” molecule refers to a substantially purified molecule.

As used herein “agent,” “compound,” and “drug” indicate a mixture of chemical compounds, a biological macromolecule, or an extract made from biological materials such as bacteria, plants, fungi, or animal (particularly mammalian) cells or tissues that are suspected of having therapeutic properties. The compound, agent or drug may be purified, substantially purified or partially purified. Additionally, an “agent,” “compound” or “drug” may be substantially pure (i.e., comprised of essentially one component).

A compound is said to be “in a form suitable for administration to an animal” when the compound may be administered to an animal by any desired route (e.g., oral, intravenous, subcutaneous, intramuscular, etc.). In particularly preferred embodiments, the compound or its active metabolites appear(s) in the blood of the animal. Administration of a compound to a pregnant female may result in delivery of the compound to the fetuses of the pregnant animal.

A “therapeutically effective” amount or dose refers to that amount of active ingredient, for example, antibodies or fragments thereof, agonists, antagonists or inhibitors of a molecule or system of interest, which ameliorates the symptoms or condition. Therapeutic efficacy and toxicity may be determined by standard pharmaceutical procedures in cell cultures or experimental animals (e.g., ED50 [the dose therapeutically effective in 50% of the population] and LD50 [the dose lethal to 50% of the population]. The dose ratio between therapeutic and toxic effects is the therapeutic index, and it can be expressed as the ratio, LD50/ED50.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

As used herein “agonist” refers to molecules or compounds which mimic the action of a “native” or “natural” compound. The present invention encompasses agonists that are homologous to these natural compounds in respect to conformation, charge or other characteristics, as well as compounds that are not homologous. Thus, agonists may or may not be recognized by, for example, receptors expressed on cell surfaces. In any event, regardless of whether the agonist is recognized by a natural compound in a manner similar to a “natural” compound or molecule, in some cases the agonist causes physiologic and/or biochemical changes within the cell (i.e., such that the cell reacts to the presence of the agonist) in the same manner as if the natural compound was present.

As used herein “antagonist” refers to molecules or compounds which inhibit the action of a “native” or “natural” compound. As used herein, “antagonist” also encompasses compounds that are homologous to these natural compounds in respect to conformation, charge or other characteristics, as well as, compounds that are not homologous. Thus, antagonists are recognized by the same or different receptors or molecules as recognized by an agonist. Antagonists may have allosteric effects which prevent the action of an agonist (e.g., by modifying a DNA adduct). In addition, in some cases, antagonists prevent the function of the agonist (e.g., by blocking a DNA repair molecule).

As used herein, “patient” and “subject” refer to a human or other animal, such simians, rodents, ovines, bovines, ruminants, lagomorphs, porcines, caprines, equines, canines, felines, ayes, etc. Preferred non-human animals are include guinea pigs and members of the Order Rodentia (e.g., mouse and rat). Thus, the compounds of the invention may be administered by human health professionals as well as veterinarians.In some preferred embodiments, a patient is treated using the methods and compositions of the present invention.

As used herein, “host” refers to a recipient cell or organism.

As used herein, “autoimmune disease” refers to any pathological condition in which an animal produces antibodies that recognize “self” antigens.

As used herein, “immunotherapy” refers to the use of therapy to improve the immune function of a subject. In particularly preferred embodiments, immunotherapy comprises the administration of compounds that stimulate the immune response to produce active immunity. However, it is not intended that the present invention be limited to any particular type of immunotherapy, as any therapy suitable for use with a particular subject is contemplated.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

“Antibody” as used herein, refers to a glycoprotein produced by B cells and plasma cells that binds with high specificity to an antigen (usually, but not always, a peptide) or a structurally similar antigen, that generated its production. Antibodies may be produced by any of the known methodologies and may be either polyclonal or monoclonal. The term antibody includes “antibody fragments” described below.

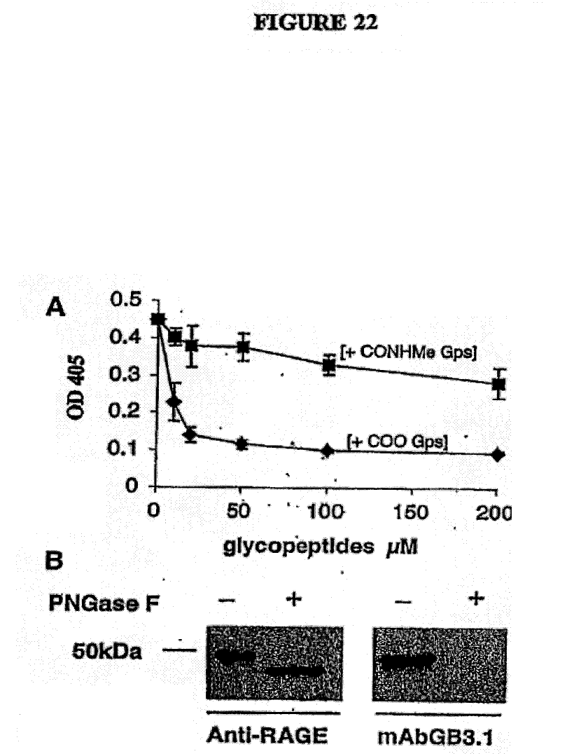

The terms “specific binding,” specifically binding” and grammatical equivalents when used in reference to the interaction of an antibody with a protein or with a carboxylated glycan mean that the interaction is dependent upon the presence of a particular structure (i.e., the antigenic determinant or epitope) on the protein or carboxylated glycan; in other words the antibody is recognizing and binding to a specific protein structure on the protein, and to a carboxyl group on the carboxylated glycan, respectively, rather than respectively to proteins or to other anionic groups on the carboxylated glycan, in general. For example, if an antibody is specific for epitope “A,” the presence of a protein containing epitope A (or free, unlabelled A) in a reaction containing labelled “A” and the antibody will reduce the amount of labelled A bound to the antibody. Similarly, if an antibody is “specific for a carboxylated glycan” (i.e., a “carboxylated glycan-specific antibody”), then the presence of unlabelled carboxylated glycan in a reaction containing a labelled carboxylated glycan will reduce the amount of labelled carboxylated glycan that is bound to the antibody, whereas the presence of unlabelled carboxylated glycan in which the carboxyl group has been modified (e.g., by reversible esterification of the carboxyl group to an alkyl, or by irreversible alkylamidation of the carboxyl group) will not substantially reduce the amount of binding of labelled carboxylated glycan that is bound to the antibody. For example, data herein shows that anti-carboxylate antibody mAbGB3.1 immunoprecipitated bovine RAGE, and the binding of amphoterin to RAGE decreases significantly in the presence of soluble carboxylated glycans or when the receptor is deglycosylated (Example 2; Example 25; FIG. 22, Panel A).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, J. et al., supra, pp 7.39-7.52 [1989]).

“Staining,” as used herein, refers to any number of processes known to those in the field that are used to better visualize a specific component(s) and/or feature(s) of a cell or cells.

“Nucleic acid sequence,” “nucleotide sequence” and “polynucleotide sequence” as used herein refer to an oligonucleotide or polynucleotide, and fragments or portions thereof, and to DNA or RNA of genomic or synthetic origin which may be single- or double-stranded, and represent the sense or antisense strand.

As used herein, the terms “oligonucleotides” and “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

The term “nucleotide sequence of interest” refers to any nucleotide sequence, the manipulation of which may be deemed desirable for any reason, by one of ordinary skill in the art. Such nucleotide sequences include, but are not limited to, coding sequences of structural genes (e.g., reporter genes, selection marker genes, oncogenes, drug resistance genes, growth factors, etc.), and of non-coding regulatory sequences that do not encode an mRNA or protein product (e.g., promoter sequence, enhancer sequence, polyadenylation sequence, termination sequence, etc.).

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product which displays modifications in sequence and or functional properties (Le., altered characteristics) when compared to the wild-type gene or gene product. It is noted that naturally-occurring mutants can be isolated; these are identified by the fact that they have altered characteristics when compared to the wild-type gene or gene product.

“Amino acid sequence,” “polypeptide sequence,” “peptide sequence,” and “peptide” are used interchangeably herein to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. The term “portion” when used in reference to an amino acid sequence refers to fragments of the amino acid sequence. The fragments may range in size from 3 amino acids to the entire amino acid sequence minus one amino acid residue.

An oligonucleotide sequence which is a “homolog” of a first nucleotide sequence is defined herein as an oligonucleotide sequence which exhibits greater than or equal to 50% identity, and more preferably greater than or equal to 70% identity, to the first nucleotide sequence when sequences having a length of 10 by or larger are compared.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects that transcription proceeds in a 5′ to 3′ direction along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “cloning” as used herein, refers to the process of isolating a nucleotide sequence from a nucleotide library, cell or organism for replication by recombinant techniques.

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed using a recombinant DNA molecule.

The term “transfection” as used herein refers to the introduction of foreign DNA into cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-CAGT-3′,” is complementary to the sequence “5′-ACTG-3′.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands may have significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary (i.e., “substantially homologous”) to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ° C. to about 20° C. to 25° C. below Tₘ. As will be understood by those of skill in the art, a stringent hybridization can be used to identify or detect identical polynucleotide sequences or to identify or detect similar or related polynucleotide sequences. Under “stringent conditions” the nucleotide sequence portions thereof, will hybridize to its exact complement and closely related sequences.

Low stringency conditions comprise conditions equivalent to binding or hybridization at 68° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 2.0×SSPE, 0.1% SDS at room temperature when a probe of about 100 to about 1000 nucleotides in length is employed.

It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) are well known in the art. High stringency conditions, when used in reference to nucleic acid hybridization, comprise conditions equivalent to binding or hybridization at 68° C. in a solution consisting of 5×SSPE, 1% SDS, 5× Denhardt's reagent and 100 μg/ml denatured salmon sperm DNA followed by washing in a solution comprising 0.1×SSPE and 0.1% SDS at 68° C. when a probe of about 100 to about 1000 nucleotides in length is employed.

When used in reference to a double-stranded nucleic acid sequence such as a cDNA or genomic clone, the term “substantially homologous” refers to any probe which can hybridize either partially or completely to either or both strands of the double-stranded nucleic acid sequence under conditions of low stringency as described above.

When used in reference to a single-stranded nucleic acid sequence, the term “substantially homologous” refers to any probe which can hybridize (i.e., it is the complement of) the single-stranded nucleic acid sequence under conditions of low stringency as described above.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀t or R₀t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization [1985]). Other references include more sophisticated computations which take structural as well as sequence characteristics into account for the calculation of Tₘ.

The term “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

“Amplification” is defined herein as the production of additional copies of a nucleic acid sequence and is generally carried out using polymerase chain reaction technologies well known in the art (see, e.g., Dieffenbach and Dveksler, PCR Primer, a Laboratory Manual, Cold Spring Harbor Press, Plainview N.Y. [1995]). As used herein, the term “polymerase chain reaction” (“PCR”) refers to the methods of U.S. Pat. Nos. 4,683,195, 4,683,202, and 4,965,188, all of which are hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and of an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double- or single-stranded DNA at or near a specific nucleotide sequence.

As used herein, the term “an oligonucleotide having a nucleotide sequence encoding a gene” means a nucleic acid sequence comprising the coding region of a gene, i.e. the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers, promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers, splice junctions, intervening sequences, polyadenylation signals, etc. or a combination of both endogenous and exogenous control elements.

Transcriptional control signals in eukaryotes comprise “enhancer” elements. Enhancers consist of short arrays of DNA sequences that interact specifically with cellular proteins involved in transcription (Maniatis et al., Science 236:1237 [1987]).

Enhancer elements have been isolated from a variety of eukaryotic sources including genes in plant, yeast, insect and mammalian cells and viruses. The selection of a particular enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York, pp. 16.7-16.8 [1989]). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene.

The term “promoter,” “promoter element,” or “promoter sequence” as used herein, refers to a DNA sequence which when placed at the 5′ end of (i.e., precedes) an oligonucleotide sequence is capable of controlling the transcription of the oligonucleotide sequence into mRNA. A promoter is typically located 5′ (i.e., upstream) of an oligonucleotide sequence whose transcription into mRNA it controls, and provides a site for specific binding by RNA polymerase and for initiation of transcription.

The term “promoter activity” when made in reference to a nucleic acid sequence refers to the ability of the nucleic acid sequence to initiate transcription of an oligonucleotide sequence into mRNA.

As used herein, the terms “nucleic acid molecule encoding,” “nucleotide encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “isolated” when used in relation to a nucleic acid, as in “an isolated oligonucleotide” refers to a nucleic acid sequence that is separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a polypeptide of interest includes, by way of example, such nucleic acid in cells ordinarily expressing the polypeptide of interest where the nucleic acid is in a chromosomal or extrachromosomal location different from that of natural cells, or is otherwise flanked by a different nucleic acid sequence than that found in nature. The isolated nucleic acid or oligonucleotide may be present in single-stranded or double-stranded form. Isolated nucleic acid can be readily identified (if desired) by a variety of techniques (e.g., hybridization, dot blotting, etc.). When an isolated nucleic acid or oligonucleotide is to be utilized to express a protein, the oligonucleotide will contain at a minimum the sense or coding strand (i.e., the oligonucleotide may be single-stranded). Alternatively, it may contain both the sense and anti-sense strands (i.e., the oligonucleotide may be double-stranded).

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” or “structural nucleotide sequence” refers to a DNA sequence coding for RNA or a protein which does not control the expression of other genes. In contrast, a “regulatory gene” or “regulatory sequence” is a structural gene which encodes products (e.g., transcription factors) which control the expression of other genes.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements include splicing signals, polyadenylation signals, termination signals, etc.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the rnRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

A “non-human animal” refers to any animal which is not a human and includes vertebrates such as rodents, non-human primates, ovines, bovines, ruminants, lagomorphs, porcines, caprines, equines, canines, felines, ayes, etc. Preferred non-human animals are selected from the order Rodentia.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

A “transformed cell” is a cell or cell line that has acquired the ability to grow in cell culture for many multiple generations, the ability to grow in soft agar and the ability to not have cell growth inhibited by cell-to-cell contact. In this regard, transformation refers to the introduction of foreign genetic material into a cell or organism. Transformation may be accomplished by any method known which permits the successful introduction of nucleic acids into cells and which results in the expression of the introduced nucleic acid. “Transformation” includes but is not limited to such methods as transfection, microinjection, electroporation, and lipofection (liposome-mediated, gene transfer). Transformation may be accomplished through use of any expression vector. For example, the use of baculovirus to introduce foreign nucleic acid into insect cells is contemplated. The term “transformation” also includes methods such as P-element mediated germline transformation of whole insects. Additionally, transformation refers to cells that have been transformed naturally, usually through genetic mutation.

As used herein, the term “kit” is used in reference to a combination of reagents and other materials. It is contemplated that the kit may include reagents such as antibodies, control proteins, as well as testing containers (e.g., microtiter plates, etc.). It is not intended that the term “kit” be limited to a particular combination of reagents and/or other materials.

The terms “link,” “conjugate,” “attach” and grammatical equivalents thereof when used in reference to a first molecule and a second molecule refer to creating a physical attachment between the two molecules, such as by covalent and/or non-covalent bonding, more preferably by covalent bonding. Methods for chemically linking molecules to each other, such as of linking carrier molecules to an agent of the invention, reporter molecule, chelator, cytotoxin, therapeutic nuclides, and nucleotide sequence are known in the art.

As used herein, the singular forms “a,” “an” and “the” include plural references unless the content clearly dictates otherwise.

### General Description of the Invention

The present invention provides methods and compositions involved in the inflammatory response. In particular, the present invention provides novel antibodies directed against novel glycans that are enriched on endothelial cell surfaces. In addition, the present invention provides methods and compositions involved in a previously unrecognized pathway of the inflammatory response. In addition, the present invention provides methods and compositions suitable to mediate the inflammatory response in various settings, as well as methods and compositions for the identification of other inflammatory response mediators. In addition, the present invention provides protein molecules capable of mediating the interaction of neutrophils with endothelial cells. These molecules, produced by neutrophils, bind to the novel carboxylated glycans on endothelial cells.

In one embodiment, the present invention provides novel glycans that are recognized by proteins produced by neutrophils. In some embodiments, these neutrophil-produced proteins are two leukocyte calcium-binding proteins. In alternative embodiments, these neutrophil-produced proteins are S100A8 and annexin I. In particularly preferred embodiments, an intact N-terminus of annexin I and heteromeric assembly of S100A8 with S100A9 (i.e., another member of the S100 family) is involved in this interaction. Thus, the present invention provides methods and compositions for the development of approaches to block acute inflammation by affecting neutrophil function and/or the production or action of proteins produced by neutrophils.

The present invention also provides antibodies that recognize novel glycans. In particular, the antibodies recognize the novel glycans associated with endothelial cells. In some embodiments, these antibodies block neutrophil binding to immobilized carboxylated glycans. In particularly preferred embodiments, the present invention provides IgG monoclonal antibodies with increased specificity and sensitivity. These antibodies provide particular advantages in various aspects of the present invention.

For example, these antibodies are more advantageous for in situ localization in tissues and for use in a variety of assays, as compared to IgM monoclonals. In particularly preferred embodiments, the monoclonal antibodies of the present invention are produced by invoking an IgG-based immune response against glycans conjugated to biotinylated diaminopyridine (BAP), and presented in multivalent arrays on streptavidin. In alternatively preferred embodiments, the antibodies of the present invention are high affinity IgG monoclonal antibodies directed against the carboxylate-containing epitope from bovine lung glycans. In still further embodiments, the present invention provides antibodies that recognize an anionic glycan modification that is highly enriched in vascular endothelial cells. As it is contemplated that these modified glycans are involved in the acute inflammatory responses via specific interactions with activated neutrophils, it is contemplated that these antibodies will find use in moderating the inflammatory response, as well as in the development of other molecules and/or compounds that are capable of moderating the response.

The present invention also provides purified human S100A8/A9 complexes and recombinant human annexin I. In some embodiments, these complexes and recombinant annexin I are capable of carboxylate-dependent binding to immobilized bovine lung carboxylated glycans. In alternative embodiments, these complexes and recombinant annexin I recognize a subset of mannose-labeled endothelial glycoproteins immunoprecipitated by mAbGB3.1. Saturable binding of S100A8/A9 complex to endothelial cells is also blocked by mAbGB3.1. Thus, the present invention provides carboxylated glycans that are important in leukocyte trafficking due to their interactions with proteins known to modulate extravasation. In addition, in alternatively preferred embodiments, the carboxylated glycans of the present invention provide a 10-fold improvement in the specific binding of amphoterin and a cell-surface signalling molecule called RAGE.

The present invention further provides methods and compositions for investigating the functions of S100A8/A9, and especially the S100A9 component. In particularly preferred embodiments, assays to assess the interaction of these complexes and components with novel carboxylated glycans on endothelial cells are provided. However, an understanding of these mechanisms is not necessary in order to use the present invention.

The present invention also provides methods and compositions for investigating the functions of annexin I. In particularly preferred embodiments, assays to assess the interaction of annexin I with novel carboxylated glycans on endothelial cells are provided. However, an understanding of these mechanisms is not necessary in order to use the present invention. In other embodiments, the recombinant full-length human annexin I provided by the present invention is capable of binding to the novel glycans of the present invention in a carboxylate dependent manner. In some embodiments, the precise epitopes recognized by annexin I and S100A8/A9, and the physiological processes mediated in vivo by annexin I and S100A8/A9 differ.

In other embodiments, the novel glycans of the present invention are capable of binding to secretions from activated neutrophils. Indeed, in other embodiments, the S100A8/A9 complexes used in the present invention are associated with the neutrophil membranes and in secretions within minutes after fMLP activation. In preferred embodiments, these proteins lack classical leader peptide sequences and transmembrane domains.

The present invention provides novel carboxylated endothelial glycans that bind to soluble leukocyte proteins which are secreted upon activation. In further, particularly preferred embodiments, these novel carboxylated endothelial glycans mediate inflammatory responses. It is further embodiments, endothelial glycoproteins carry the novel carboxylated glycans of the present invention. In other embodiments, the present invention provides binding partners involved in mediating the functions of S100A8/A9 and annexin I. Furthermore, in other embodiments, the present invention provides signaling mechanisms by which these proteins bring about their inflammatory responses in vivo.

In further embodiments, the present invention provides compositions and methods to block and/or modulate functional interactions in a carbohydrate-specific manner. In particular, the present invention provides means that block and/or modulate interactions of the novel sugar chains provided by the invention with such compounds as glycopeptides, mimetics, amphoterin, annexin I, and S100 proteins.

Thus, the present invention provides methods and compositions involved in the inflammatory response. In particular, the present invention provides novel antibodies directed against novel glycans that are enriched on endothelial cell surfaces. In addition, the present invention provides methods and compositions involved in a previously unrecognized pathway of the inflammatory response. In addition, the present invention provides methods and compositions suitable to mediate the inflammatory response in various settings, as well as methods and compositions for the identification of other inflammatory response mediators.

Leukocyte recruitment into sites of inflammation is a multistep process of interrelated events mediated by complex and overlapping functions of multiple adhesion molecules (Butcher, Cell 67:1033 [1991]; and Springer, Aim. Rev. Physiol., 57:827 [1995]). Selectins mediate the initial rolling and tethering of circulating neutrophils. L-selectin is constitutively expressed by most leukocytes and P-and E-selectins are expressed by activated endothelial cells following exposure to inflammatory conditions (Kansas, Blood 88:3259 [1996]; Lowe and Ward, J. Clin. Invest., 99:822 [1997]; and McEver and Cummings, J. Clin. Invest., 100:485 [1997]). Concomitant up-regulation of β2 integrins on the neutrophils are largely responsible for stronger adhesive interactions with intercellular adhesion molecules (ICAM-1 and ICAM2). ICAM-1 is constitutively expressed at low levels by endothelial cells, but is rapidly upregulated during inflammation (Springer, supra; and Kansas, supra).

Although rolling and firm adhesion of leukocytes are well understood, there is much less information regarding the in vivo mechanisms mediating transmigration. Several in vitro and in vivo studies have shown that platelet-endothelial cell adhesion molecule-1 (PECAM-1; CD31) is critically involved in transendothelial migration (Newman, J. Clin. Invest., 99:3 [1997]). Interestingly, antibodies against PECAM-1 inhibit leukocyte extravasation from mesenteric vessels (Wakelin et al., J. Exp, Med., 184:229 [1996]; and Bogen et al., J. Exp. Med., 179:1059 [1994]), similar to the effects of mAbGB3.1 observed as described herein. However, the expression patterns of CD31 on the surface of most leukocytes, platelets and intercellular junctions of endothelial cells (Newman, supra) is different from that of the GB3.1 antigens. Collectively, the results provided herein indicate the carboxylated glycans participate in yet another set of novel mechanisms involved in the regulation of acute inflammation.

These novel carboxylated glycans were developed in work involving a library of bovine lung oligosaccharides. These novel carboxylated N-glycans were found to be constitutively expressed on endothelial cells and mediate acute inflammatory responses. In order to further characterize these glycans and their functions, monoclonal antibodies were produced.

Indeed, IgG monoclonal antibodies were produced against the modification by immunization with biotinylated aminopyridine-derivatized glycans enriched for the anionic species, and screening for antibodies whose reactivities were abrogated by carboxylate-neutralization of bovine lung glycopeptides. One such antibody (mAbGB3.1) was inhibited by carboxylated bovine lung glycopeptides and other multi-carboxylated molecules, but not by glycopeptides in which the carboxylate groups were modified. The antibody recognized an epitope constitutively expressed on bovine, human, and other mammalian endothelial cells. Stimulated, but not resting neutrophils, bound to immobilized bovine lung glycopeptides in a carboxylate-dependent manner. The binding of activated neutrophils to immobilized bovine lung glycopeptides was inhibited both by mAbGB3.1 and by soluble glycopeptides in a carboxylate-dependent manner. The antibody also inhibited extravasation of neutrophils and monocytes in a murine model of peritoneal inflammation (i.e., neutrophil and monocyte efflux across the murine mesenteric venular endothelium was inhibited). This inhibition of cell trafficking correlated with increased sequestration but reduced transmigration of leukocytes that were found to be adherent to the endothelium of the mesenteric venules. Taken together, these results indicate that these novel carboxylated N-glycans are constitutively expressed on vascular endothelium, and participate in acute inflammatory responses by interaction with activated neutrophils.

In order to identify molecules which mediated these interactions, binding proteins were isolated from bovine lung by their differential affinity for carboxylated or neutralized glycans. Two leukocyte calcium-binding proteins that bound in a carboxylate-dependent manner were identified as S100A8 and annexin I. An intact N-terminus of annexin I, and heteromeric assembly of S100A8 with S100A9 (another member of the S100 family) appeared necessary for this interaction. A monoclonal antibody to S100A9 blocked neutrophil binding to immobilized carboxylated glycans. Purified human S100A8/A9 complex and recombinant human annexin I exhibited carboxylate-dependent binding to immobilized bovine lung carboxylated glycans, and recognized a subset of mannose labeled endothelial glycoproteins immunoprecipitated by mAbGB3.1. Saturable binding of S100A8/A9 complex to endothelial cells was also blocked by mAbGB3.1. These results indicate that the carboxylated glycans play important roles in leukocyte trafficking by interacting with proteins known to modulate extravasation.

The present invention provides methods involving two soluble proteins of predominant leukocyte origin, which are externalized upon activation, and are known to modulate neutrophil and monocyte extravasation. In particular, the present invention provides means for the specific interaction with the novel carboxylated glycans of bovine lung.

In addition, the present invention provides means to identify and modulate the activity of ligands that bind to carboxylated sugar chains. As indicated herein, the embryonic neurite growth-promoting protein amphoterin binds to carboxylated N-glycans identified on mammalian endothelial cells. Since amphoterin is a ligand for the Receptor for Advanced Glycation End products (RAGE), and the ligand-binding V-domain of the receptor contains two potential N-glycosylation sites, it was hypothesized by the inventors that N-glycans on RAGE mediate its interactions with amphoterin. As discussed herein, anti-carboxylate antibody mAbGB3.1 immunoprecipitates bovine RAGE, and PNGase F treatment reduces its molecular mass by 4.5 kD, indicating that the native receptor is a glycoprotein. The binding potential of amphoterin to RAGE decreases significantly in the presence of soluble carboxylated glycans or when the receptor is deglycosylated. Oligosaccharide analysis shows that RAGE contains complex type anionic N-glycans with non-sialic acid carboxylate groups, but not the HNK-1 (3-sulfoglucuronyl β1-3 galactoside) epitope.

Consistent with the functional localization of RAGE and amphoterin at the leading edges of developing neurons, mAbGB3.1 stains axons and growth cones of mouse embryonic cortical neurons, and inhibits neurite outgrowth on amphoterin matrix. The carboxylated glycans themselves promote neurite outgrowth in embryonic neurons and RAGE-transfected neuroblastoma cells. This outgrowth requires full length, signaling-competent RAGE, since cells expressing cytoplasmic domain-deleted RAGE are unresponsive. These results indicate that carboxylated N-glycans on RAGE play an important functional role in amphoterin-RAGE mediated signaling.

The anti-carbohydrate antibody, mAbGB3.1 generated against carboxylate-enriched desialylated bovine lung N-glycans provided by the present invention facilitates identification of targets for treatment of disease. Antibody reactivity is abrogated by carboxylate-methylamidation of bovine lung glycopeptides, showing that it recognizes the non-traditional carboxylated epitopes on bovine lung N-linked oligosaccharides. Thus, the antibody provides a tool to identify carboxylated N-glycans on candidate proteins.

However, it is not intended that the present invention be limited to the antibodies described in detail herein, as it is contemplated that other entities will find use in blocking interactions of the novel carboxylated epitopes on N-glycans described herein. For example, it is contemplated that in addition to annexins and amphoterin, other bioactive proteins will find use in the regulation of multiple pathological processes.

It is contemplated that the means provided by the present invention will find use in treatment and prevention of numerous diseases and pathological conditions, including but not limited to Crohn's disease, tumor growth and metastasis, complications associated with diabetes, Alzheimer's disease, dementia, inflammation associated with atherogenesis, periodontal disease, skin immune responses, septic shock, heart disease, chronic inflammation and acute inflammation. Indeed, the present invention provides means to identify ligands involved in various pathological processes, as well as antibodies that are capable of blocking ligand binding and thereby prevent, impede, and/or reverse the pathological effects of RAGE-ligand interactions.

### DETAILED DESCRIPTION OF THE INVENTION

The present invention provides methods and compositions involved in the inflammatory response. In particular, the present invention provides novel antibodies directed against novel glycans that are enriched on endothelial cell surfaces. In addition, the present invention provides methods and compositions involved in a previously unrecognized pathway of the inflammatory response. Furthermore, the present invention provides methods and compositions suitable to mediate the inflammatory response in various settings, as well as methods and compositions for the identification of other inflammatory response mediators. In addition, the present invention provides protein molecules capable of mediating the interaction of neutrophils with endothelial cells. These molecules, produced by neutrophils, bind to the novel carboxylated glycans on endothelial cells.

The invention is further described below under (A) Purifying carboxylated glycans, (B) Identifying agents that reduce specific binding of a polypeptide to a carboxylated glycan, (C) Antibodies and cell lines producing antibodies, (D) Agents useful in the invention's methods, (E) Reducing extravasation of leukocytes, (F) Reducing adherence of leukocyte cells to endothelial cells, and (G) Reducing inflammation and cancer.

### A. Purifying Carboxylated Glycans

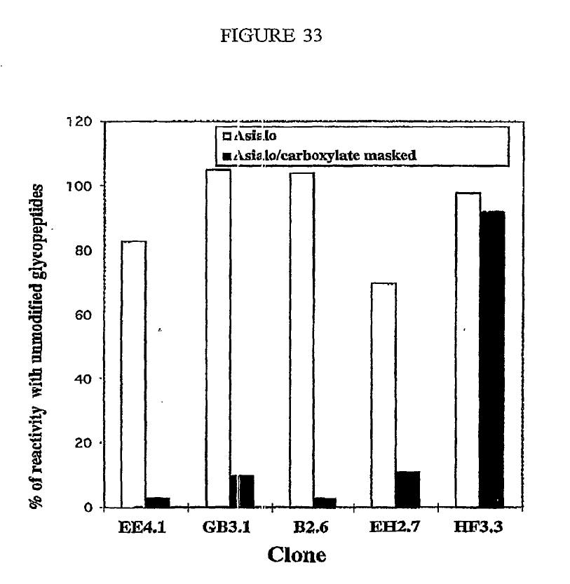

The invention provides methods for purifying a carboxylated glycan. In one embodiment, the invention provides a method for purifying a carboxylated glycan, the method comprising: a) providing: i) a molecule comprising a carboxylated glycan; biotinylated diamino pyridine (BAP); and an exoglycosidase; b) conjugating the molecule to the BAP to produce a BAP-glycan conjugate; c) treating the BAP-glycan conjugate with the exoglycosidase to produce a first treated BAP-glycan conjugate comprising a first anionic BAP-glycan conjugate having from 1 to 2 negative charges per molecule, more preferably having 1 negative charge per molecule; and d) isolating the first anionic BAP-glycan conjugate, thereby purifying a carboxylated glycan. These methods are useful for isolating carboxylated glycans that may be used to raise antibodies, and in particular IgG antibodies such as the exemplary mAbEE4.1, mAbGB3.l, mAbB2.6, and mAbEH2.7 antibodies described herein. Furthermore, these methods are also useful for generating carboxylated glycans that are soluble or that may be immobilized to a solid surfaces for use in competition assays (see for example FIG. 33).

The term “glycan” refers to a polymer composed wholly or largely of sugars and their derivatives (e.g., amino sugars and sugar alcohols). The term “glycan” also refers to the carbohydrate parts of other types of molecule (e.g., proteins, lipids, etc.) to which sugars are covalently linked, whether as monosaccharides, oligosaccharides or polysaccharides, to form glycoconjugtes. The linkage of sugar is preferably as a glycoside and the remainder of the molecule to which the sugar is linked is the “aglycone.” Glycans may be branched, their glycosidic bonds may be variously α or β anomers, their sugars may be pyranoses or furanoses, and the positions of linkage may vary.

The terms “carboxylated glycan,” “COO⁻glycan,” and “carboxylated N-glycan” are used interchangeably herein to refer to a glycan molecule that is “enriched” for carboxyl groups, i.e., a molecule that contains a number of carboxyl groups that is at least 10% more, more preferably at least 50% more, yet more preferably at least 100% more, even more preferably at least 150% more, than the number of sialic acid groups, uronic acid groups, sulphate groups, or phosphate groups, as compared to the numbers of corresponding groups in the molecule from which the glycan molecule was purified. In a most preferred embodiment, the carboxylated glycan contains carboxyl groups in the absence of any sialic acid groups, uronic acid groups, sulphate groups, and phosphate groups. A carboxylated glycan may be identified by methods disclosed herein. For example, a reduction in binding of a carboxylated glycan-specific antibody to an immobilized molecule in the presence of a soluble carboxylated glycan and the absence of a reduction in binding of a carboxylated glycan-specific antibody to the immobilized molecule in the presence of a soluble carboxylated glycan that has been modified (e.g., by reversible esterification of the carboxyl group to an alkyl, or by irreversible alkylamidation of the carboxyl group) (Example 2) so as to neutralize the carboxyl group of the carboxylated glycan indicates that the immobilized molecule contains a carboxylated glycan. For example, data herein shows that soluble COO⁻ glycopeptide, but not CONHMe-glycopeptide species blocked binding of carboxylated glycan antibodies to immobilized RAGE (Examples 25; FIG. 22; Panel A).

As disclosed herein, the molecules that contain the carboxylated glycans of the invention may be glycopeptides or polysaccharides. When the carboxylated glycans are prepared from samples that additionally contain proteins (such as the lung cell extracts disclosed herein), one of skill in the art appreciates that these samples may preferably be treated with a proteinase to hydrolyze polypeptides prior to or after the step of conjugating the glycopeptide or polysaccharide to BAP. The terms “proteinase” and “protease” as used herein refer to an enzyme that degrades proteins by hydrolyzing peptide bonds between amino acid residues.

The term “glycosidase” refers to an enzyme that catalyzes the cleavage of a glycoside linkage. Glycosidic linkages are formed by the reaction of sugars (such as monosaccharides) with alcohols (to form O-glycosides), amines (to form N-glycosides), or other molecules. Glycosidases include exoglycosidases and endoglycosidases. An “exoglycosidase” refers to an enzyme that cleaves the glycosidic linkage of unsubstituted (or terminal) sugar residues. Exoglycosidases are exemplified by, but not limited to, Arthrobacter ureafaciens sialidase, jack bean β-N-acetylhexosaminidase, bovine testicular β-galactosidase, coffee-bean α-galactosidase, jack bean α-mannosidase, alpha1,3/4-fucosidase, alphal,6-fucosidase, beta-glucuronidase, beta-xylosidase, alpha-N-acetylgalactosaminidase, alpha2,6sialidase, and alpha2,3sialidase.

One of skill in the art that enzymes other than (or as well as) exoglycosidases may be used, so long as these enzymes remove moieties other than carboxylated sugars. Exemplary equivalent enzymes include, without limitation, endoglycosidases and sulfatases. An “endoglycosidase” refers to an enzyme which cleaves the glycosidic linkage of substituted (or internal) residues, and is exemplified by endoglycosidase H, endoglycosidase S, endo-neuraminidase, endo-b-galactosidase, endo 1,2 alpha-mannosidase, chondroitinase ABC, chondroitinase AC, heparinase, and heparatinase. “Sulfatases” are exemplified by N-acetylglucosamine 3-O-sulfatase, N-aceylglucosamine 6-O-sulfatase, and iduronic acid 2-O-sulfatase.

One of skill in the art knows that equivalents to biotinylated diamino pyridine (BAP) may be used. These equivalents include, for example, compounds that have a free amino group that reacts with a reducing end of sugars. Preferably, the compound is fluorescent, and has a biotin for streptavidin binding.

In one embodiment, the purification method involves repeating the exoglycosidase treatment and/or isolation of the anionic BAP-carboxylated glycan conjugate to further enrich for the carboxylated glycan. Thus, in one preferred embodiment, the method further comprises the steps of: e) treating the first anionic BAP-glycan conjugate produced in step c) or step d) with an exoglycosidase to produce a second anionic treated BAP-glycan conjugate comprising a second anionic BAP-glycan conjugate having from 1 to 2, more preferably 1, negative charges per molecule; and f) isolating the second anionic BAP-glycan conjugate, thereby purifying a carboxylated glycan. Preferably, the BAP-glycan conjugate produced in step e) contains 1 negative charge per molecule. In a more preferred embodiment, the method further comprises repeating steps e) and f) from 1 to 10 times, more preferably, from 1 to 5 times, yet more preferably from 1 to 3 times, and most preferably from 1 to 2 times. In an alternative embodiment, the molecule is a glycoprotein or polysaccharide.

In one embodiment, the isolating comprises fractionating by ion exchange chromatography. The term “fractionating” or “separating” by ion exchange chromatography” refers to separating substances based on ionic interactions and/or hydrophobicity. Exemplary ion exchange chromatograph includes anion exchange chromatography such non-HPLC columns of DEAE cellulose, DEAE-sephadex, DEAE-sepharose, and QAE-sephadex, as well as HPLC columns of DEAE, mono-Q, IEC DEAE-825 (Shodex), HYDROCELL DEAE 1000, and TSK DEAE-5PW1.

The invention also provides a method for purifying a carboxylated glycan, the method comprising: a) providing a molecule comprising a carboxylated glycan, optionally treating said molecule with a proteinase to hydrolyze polypeptides and to generate a first sample containing glycopeptide or polysaccharide; b) isolating from the molecule a first anionic glycan containing from 1 to 5, more preferably from 1 to 4, and most preferably from 1 to 3 negative charges; and c) desialylating the isolated first anionic glycan to produce a desialylated anionic glycan containing from 1 to 4 negative charges, thereby purifying a carboxylated glycan. One of skill appreciates that steps c) and d) may be reversed in sequence. These methods are useful for isolating carboxylated glycans that may be used to raise antibodies. However, unlike the method discloses above which involves conjugation to BAP, this method is less likely to generate an IgG antibody that is specific for the carboxylated glycan. Furthermore, these methods are also useful for generating carboxylated glycans that are soluble or that may be immobilized to a solid surfaces for use in competition assays (see for example FIG. 33).

The term “desialylating” as used herein refers to removing sialic acid. This may be achieved using known methods such as by using commercially available sialidase enzyme such as Arthrobacter ureafaciens sialidase, alpha2,6sialidase, and alpha2,3 sialidase, or by acid treatment as described in Example 1 which discloses that glycopeptides generated from bovine lung acetone powder were desialylated by treatment with 10 mM HCl, 30 min at 100° C. generating “asialo-COO⁻glycopeptides”.

In one embodiment, the method further comprises d) isolating from the first desialylated anionic glycan a second anionic glycan containing from 1 to 5, more preferably from 1 to 4, and most preferably from 1 to 3 negative charges, thereby purifying a carboxylated glycan. In an alternative embodiment, the method further comprises prior to step a) the step of treating the molecule with a proteinase enzyme.

As described herein, carboxylated glycans are present on RAGE, a well-documented signal-transducing receptor for amphoterin (Hori et al., supra; Huttunen et al., J. Biol. Chem., 274:19919-199124 [1999]; and Taguchi et al., supra). Furthermore, as indicated herein, the carboxylated glycans mediate RAGE-amphoterin interactions.

In addition, carboxylated glycans themselves promote neurite outgrowth in embryonic neurons and RAGE-transfected neuroblastoma cells. This outgrowth requires full length, signaling-competent RAGE, since cells expressing cytoplasmic domain-deleted RAGE are unresponsive. These results indicate that the glycans induce or stabilize a multivalent protein complex through homophile association (Hakomori et al., Ann. N.Y. Acad. Sci., 845:1-10 [1998]), or by forming a RAGE-amphoterin-immobilized glycan multivalent complex. It is contemplated that a variable aggregation threshold may be crucial for differential signaling in vivo. In fact, a novel paradigm for supermolecular assembly and signal transduction based on cross-linking of multivalent carbohydrates with multivalent lectins has been recently proposed, based on studies of receptor clustering involving endogenous galectin-1 and its counter receptors on human T cells (See, Sacchettini et al., Biochem., 40:3009-3015 [2001]). N-glycans are known to modulate signaling (Mies et al., Immun., 9:881-890 [1998]; Hennet et al., Proc. Natl. Acad. Sci. USA 95:4504-4509 [1998]; and Priatel et al., Immun., 12:273-283 ]2000]). A GlcNAc transferase differentially modulates Notch-1 binding to its ligands, Delta and Serrate (Moloney et al., Nature 406:369-375 [2000]). Deficiency of β1,6 GlcNAc transferase (Mgat-5) lowers T-cell activation threshold by enhancing T-cell receptor clustering (Demetriou et al., Nature 409:733-739 [2001]), and multivalent galectin-Mgat-5 modified glycoprotein lattices limit agonist-mediated clustering. The present invention provides additional effects of glycans on apoptosis, immunomodulation and immune response.

B. Identifying Agents that Reduce Specific Binding of a Polypeptide to a Carboxylated Glycan

The invention further provides methods for identifying a test agent as reducing specific binding of a polypeptide to a carboxylated glycan. In one preferred embodiment, the method comprises: a) providing: i) a carboxylated glycan purified by the above-described methods; an antibody that specifically binds to the carboxylated glycan; and a test agent; b) contacting the purified carboxylated glycan, the antibody, and the test agent; and c) detecting a reduction in the level of binding of the antibody to the carboxylated glycan in the presence of the test agent compared to in the absence of the test agent, thereby identifying the test agent as reducing specific binding of a polypeptide to a carboxylated glycan.

The terms “reducing binding” and “reduces binding” when in reference to the binding of a first molecule (e.g., polypeptide, antibody, etc.) to a second molecule (e.g., carboxylated glycan) means that the quantity of specific binding is reduced by any amount that is statistically significant using any art-accepted statistical method of analysis. Preferably the quantity of binding is reduced by at least 10%, more preferably by at least 50%, yet more preferably by at least 75%, even more preferably by at least 90%. In a most preferred embodiment, the quantity of binding is undetectable.

In one embodiment, the invention's methods employ a purified carboxylated glycan that is attached to a solid surface. The terms “solid surface” and “solid support” are used interchangeably to refer to any material that is in a solid state, such as a glass bead, planar glass, controlled pore glass, plastic, porous plastic, metal, or resin to which the carboxylated glycan may be adhered. One of skill will appreciate that the solid supports may be derivatized with functional groups (e.g., hydroxyls, amines, carboxyls, esters, and sulfhydryls) to provide reactive sites for the attachment of linkers or the direct attachment of the component(s). Adhesion of the carboxylated glcyans to the solid support may be direct or indirect, covalent or non-covalent. For example, the carboxylated glycans may be adhered to the solid surface via immobilized antibodies or other specific binding proteins, a biotin/streptavidin system, metal-chelating Langmuir-Blodgett films, or metal-chelating self-assembled monolayers.

In another embodiment of a method for identifying a test agent as reducing specific binding of a polypeptide to a carboxylated glycan, the method comprises a) providing: i) a carboxylated glycan purified by the above described methods, ii) leukocyte cells; and iii) a test agent; b) contacting the purified carboxylated glycan, the leukocyte cells, and the test agent; and c) detecting a reduction in the level of adhesion of the leukocytes to the purified carboxylated glycan in the presence of the test agent compared to in the absence of the test agent, thereby identifying the test agent as reducing specific binding of a polypeptide to a carboxylated glycan.

The term “reduction in the level of adhesion of leukocytes to a carboxylated glycan” refers to a reduction in the quantity of adhesion of leukocytes to a carboxylated glycan by any amount that is statistically significant using any art-accepted statistical method of analysis. Preferably the quantity of adhesion is reduced by at least 10%, more preferably by at least 50%, yet more preferably by at least 75%, even more preferably by at least 90%. In a most preferred embodiment, the quantity of binding is undetectable. Methods for assaying the level of adhesion of leukocytes to molecules and cells are known in the art and exemplified herein (Example 9).

It may also be desirable to further confirm the identity of a test agent as reducing specific binding of a polypeptide to a carboxylated glycan using additional methods, comprising: a) providing: i) a monolayer of endothelial cells; ii) leukocyte cells; and a test agent; b) contacting the endothelial cells, the leukocyte cells, and the test agent; and c) detecting a reduction in the level of transmigration of the leukocyte cells on the monolayer of endothelial cells in the presence of the test agent compared to in the absence of the test agent. Alternatively, or additionally, confirmation of the identity of the test agent may be further corroborated by genetically engineering a host cell to express a carboxylated glycan (e.g., RAGE) and determining whether the test agent reduces binding of amphoterin to the expressed RAGE as measured by, for example, a reduction in NFκβ signalling by RAGE.

### C. Antibodies and Cell Lines Producing Antibodies

The invention further provides antibodies specific for a carboxylated glycan, such as the monoclonal IgG antibodies mAbEE4.1, mAbGB3.1, mAbB2.6, and mAbEH2.7 that are specific for carboxylated glycans isolated from lung tissue, and that are produced by the hybridoma cell lines EE4.1, GB3.1, B2.6, and E112.7, respectively.

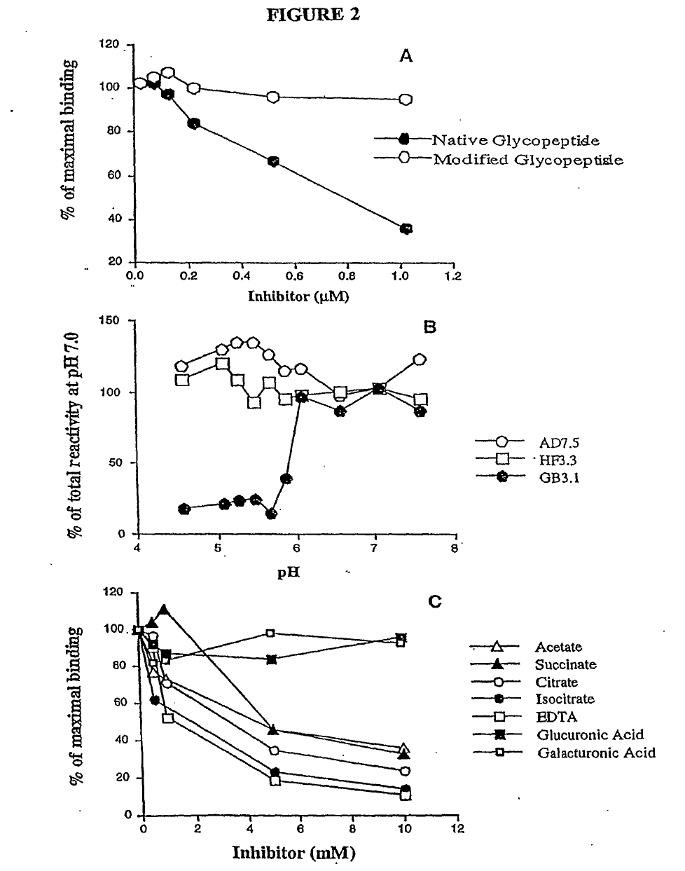

In one preferred embodiment, the binding of the carboxylated glycan-specific antibody to the carboxylated glycan is reduced by a carboxylated glycan, and the binding is not reduced by a carboxylate-neutralized glycan. The term “not reduced” when in reference to the binding of an antibody to a carboxylated glycan means that the quantity of binding of the antibody to the carboxylated glycan is not reduced by a statistically significant amount using any art-accepted statistical method of analysis. The term “carboxylate-neutralized glycan” refers to a glycan in which the anionic charge on a carboxylate group is neutralized either reversibly or irreversibly. Methods for neutralizing carboxylate charges are known in the art and exemplified herein. For example, reversible neutralization may be achieved by alkylamidation such as by using methylamine, ethylamine, etc., while irreversible neutralization may be achieved by alkylesterification such as by using methylamine, ethylamine, propylamine, etc. (Example 2). For example, data herein demonstrates that binding of mAbGB3.1 to immobilized BSA neoglycoproteins could be blocked by asialo-COO⁻glycopeptides in solution, but not by asialo-CONHMe-glycopeptides (FIG. 2, Panel A).

In another embodiment, the carboxylated glycan-specific antibody does not specifically bind to glucuronic acid, galacturonic acid, sialic acid, lactic acid, pyruvic acid, or uronic acid. The term “not specifically bind” when in reference to an antibody and molecule means that there is no between the antibody and molecule and/or binding is not specific to a particular epitope on the molecule, but rather the antibody binds to the molecule in general. For example, data herein demonstrates that glucuronic, and galacturonic acids did not inhibit binding of mABGB3.1 to carboxylated glycans purified from bovine lung (Example 11; FIG. 2, Panel C).

In an alternative embodiment, the carboxylated glycan-specific antibody does not specifically bind to a sulfated glycan. For example, data using Western blots as disclosed herein shows that mAbGB3.1 did not cross-react with sulfated glycans such as thyroglobulin, which carries terminal Gal-3-sulfate and internal GlcNAc-6-sulfate, and neural cell adhesion molecule (N-CAM), which is known to express the HNK-1 epitope carrying a terminal glucuronic acid-3-sulfate.

In another alternative, the carboxylated glycan-specific antibody does not specifically bind to a glycosaminoglycan, such as chondrosamine, chondroitin sulfate, chondroitin sulfate tetramer, chorndroitin sulfate octamer, hyaluronic acid tetramer, hyaluronic acid octamer, heparin, or heparin sulfate. Data herein shows that chondrosamine, chondroitin sulfate (tetramer and octamer), and hyaluronic acid (tetramer and octamer) at 1 to 5 mM also did not inhibit binding, indicating that glycosaminoglycan-like epitopes do not cross react (Example 3).

In another embodiment, the carboxylated glycan-specific antibody does not specifically bind to a phosphorylated sugar (such as glucose-l-phosphate, glucose-6-phosphate, mannose-6-phosphate, galactose-6-phosphate, glucose-N-acetyl-1-phosphate, and glucose-N-acetyl-6-phosphate) or to a sulfated sugar (such as glucose-6-sulfate and galactose-6-sulfate). Data herein shows that reactivity of the antibody in the ELISA assay was unaffected by sulfated or phosphorylated sugars (Example 3).]

In addition to antibodies specific for carboxylated glycans, the invention provides antibodies that are specific for polypeptides to which the carboxylated glycans bind. For example, monoclonal antibodies directed against S100A9 are also described herein. These antibodies blocked neutrophil binding to immobilized carboxylated glycans. Carbohydrate-specific monoclonal antibodies often tend to be low affinity IgM reagents which can show significant cross-reactivities amongst various glycans (Hakomori, Chem. Phys. Lipids 42:209 [1989]; Matsuda and Kabat, J. Immunol., 142:863 [1989]; and Stein et al., J. Immunol., 128:1350 [1989]), and are technically difficult to purify and stabilize. Thus, the present invention provides IgG monoclonal antibodies with increased specificity and sensitivity, as these antibodies are more advantageous for in situ localization in tissues and for use in a variety of assays. During the development of the present invention, it had been noted that glycans conjugated to biotinylated diaminopyridine (BAP), and presented in multivalent arrays on streptavidin can evoke an IgG immune response in mice (Rothenberg et al., Proc. Natl. Acad. Sci USA 90:11939; and Toomre et al., Glycobiol., 4:653 [1994]; See also, U.S. Pat. No. 5,449,781 to Varki et al., the entirety of which is hereby incorporated by reference). Taking advantage of this concept, it was possible to generate high affinity IgG monoclonal antibodies directed against the carboxylate-containing epitope from bovine lung glycans. As described in greater detail herein, one of these antibodies was used to demonstrate that this anionic modification is highly enriched in vascular endothelial cells, and participates in acute inflammatory responses by interacting specifically with activated neutrophils.

Indeed, in the development of the present invention, a novel approach was used to generate IgG monoclonal antibodies which detect unusual carboxylated N-glycans that are predominantly localized in the vascular endothelium of a variety of mammalian tissues. The vascular endothelium is a dynamic, and complex system that possesses many secretory, synthetic, immunologic and metabolic functions (Clines et al., Blood 91:3527 [1998]). The contribution of carbohydrates to endothelial function is best exemplified by the selectin family of adhesion molecules, which recognize sialyl Lewis^{x} and sialyl Lewis^{a}-containing structures (Kansas, supra, Lowe and Ward, supra; and McEver and Cummings, supra). Also, unusual anionic oligosaccharides such as sialyl Lewis^{x/a} as well as sulfosialyl Lewis^{x/a}, Man-6-phosphate (Kornfeld, Biochem. Soc. Trans., 18:367 [1990]), polysialic acid (Rutishauser, Curr. Opin. Cell Biol., 8:679 [1996]) and GalNAc-4-sulfate (Baenziger, Endocrinol., 137:1520 [1996]), have been noted to be more involved in mediating specific biological roles than the more common neutral glycans. The predominant localization of these novel carboxylated glycans on the vascular endothelium therefore raised the intriguing possibility that they could participate in endothelial functions, or in interactions with cells or proteins in the blood. The functional studies described herein clearly indicate that they can mediate interactions with activated neutrophils, and modulate inflammatory responses.

The antibodies used in the methods invention may be prepared using various immunogens. In one embodiment, the immunogen is a novel carboxylated glycan may be used as an immunogen) to generate antibodies that recognize novel glycans involved in such processes as the inflammatory response. Such antibodies include, but are not limited to polyclonal, monoclonal, chimeric, humanized, human, subhuman, single chain, antibody fragments such as Fab fragments and peptides coding for a single complementarity-determining region (CDR), and an Fab expression library.

A “chimeric antibody” is a recombinant protein that contains the variable domains and complementary determining regions derived from an antibody of one animal species (e.g. rodent), while the remainder of the antibody molecule is derived from an antibody from another animal species (e.g., human). Techniques for constructing chimeric antibodies are well-known to those of skill in the art. For example, Leung et al., Hybridoma 13:469 (1994), describe producing an LL2 chimera by combining DNA sequences encoding the V_{K} and V_{H} domains of LL2 monoclonal antibody with respective humanx and IgG₁ constant region domains. This publication also provides the nucleotide sequences of the LL2 light and heavy chain variable regions, V_{K} and V_{H}, respectively.

A “humanized antibody” is a recombinant protein in which complementarity determining regions of a monoclonal antibody from one animal species (e.g., murine) have been transferred from heavy and light variable chains of the murine immunoglobulin into a human variable domain. For example, a humanized monoclonal antibody may contain mouse complementarity determining regions that are transferred from heavy and light variable chains of the mouse immunoglobulin into a human variable domain, followed by the replacement of some human residues in the framework regions of their murine counterparts. Humanized monoclonal antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323(1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993). The publication of Leung et al., Mol. Immunol. 32:1413 (1995), describes the construction of humanized LL2 antibody.

In another embodiment, an antibody of the present invention is a “subhuman primate antibody” i.e., an antibody that is raised in a non-human primate. General techniques for raising therapeutically useful antibodies in baboons may be found, for example, in Goldenberg et al., international patent publication No. WO 91/11465 (1991), and in Losman et al., Int. J. Cancer 46: 310 (1990).

In another embodiment, an antibody of the present invention is a “human antibody.” Such antibodies may be obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994).

In an additional embodiment of the invention, monoclonal antibodies can be produced in germ-free animals utilizing recent technology (See e.g., PCT/US90/02545). According to the invention, “human antibodies” may be used and can be obtained by using human hybridomas (Cote et al., Proc. Natl. Acad. Sci. U.S.A. 80:2026-2030 [1983]) or by transforming human B cells with EBV virus in vitro (Cole et al., in Monoclonal Antibodies and Cancer Therapy, Alan R. Liss, pp. 77-96 [1985]).

Various procedures known in the art may be used for the production of polyclonal antibodies. For the production of antibody, various host animals can be immunized by injection with the peptide corresponding to a carboxylated glycan epitope or any other molecule of interest in the present invention, including but not limited to rabbits, mice, rats, sheep, goats, etc. In one preferred embodiment, the peptide is conjugated to an immunogenic carrier (e.g., diphtheria toxoid, bovine serum albumin (BSA), or keyhole limpet hemocyanin [KLH]). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward carboxylated glycans or other molecules of interest in the present invention, any technique that provides for the production of antibody molecules by continuous cell lines in culture may be used (See, e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature 256:495-497 [1975]), as well as the trioma technique, the human B-cell hybridoma technique (See e.g., Kozbor et al. Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In some particularly preferred embodiments of the present invention, the present invention provides monoclonal antibodies of the IgG class. In other particularly preferred embodiments, the monoclonal antibodies of the present invention are produced using biotinylated aminopyridine-derivatized glycans enriched for the anionic species.

According to the invention, techniques described for the production of single chain antibodies (U.S. Pat. No. 4,946,778; herein incorporated by reference) can be adapted to produce single chain antibodies that specifically recognize a molecule of interest (e.g., a carboxylated glycan, as described herein). An additional embodiment of the invention utilizes the techniques described for the construction of Fab expression libraries (Huse et al., Science 246:1275-1281 [1989]) to allow rapid and easy identification of monoclonal Fab fragments with the desired specificity for a particular carboxylated glycan.

The term “antibody fragment” includes a portion of the antibody that contains the idiotype (antigen binding region) of the antibody molecule. Such fragments include but are not limited to: the F(ab′)2 fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods are described, for example, by Goldenberg, U.S. Pat. Nos. 4,036,945 and 4,331,647 (each incorporated in its entirety by reference) and references contained therein. Also, see Nisonoff et al., Arch Biochem. Biophys. 89:230 (1960); Porter, Biochem. J. 73:119 (1959), Edelman et al., in METHODS iN ENZYMOLOGY VOL. 1, page 422 (Academic Press 1967), and Coligan at pages 2.8.1-2.8.10 and 2.10.-2.10.4. Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V.sub.H and V.sub.L chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, for example, Sandhu, supra. In one embodiment, the Fv fragments comprise V_{H} and V_{L} chains which are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing sFvs are described, for example, by Whitlow et al., Methods: A Companion to Methods in Enzymology 2:97 (1991). Also see Bird et al., Science 242:423 (1988), Ladner et al., U.S. Pat. No. 4,946,778, and Pack et al., Bio/Technology 11:1271 (1993).

Another form of an “antibody fragment” is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enymology 2:106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

In the production of antibodies, screening for the desired antibody can be accomplished by techniques known in the art (e.g., radioimmunoassay, ELISA [enzyme-linked immunosorbent assay], “sandwich” immunoassays, immunoradiometric assays, gel diffusion precipitin reactions, immunodiffusion assays, in situ immunoassays [using colloidal gold, enzyme or radioisotope labels, for example], Western Blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. (As is well known in the art, the immunogenic peptide should be provided free of the carrier molecule used in any immunization protocol. For example, if the peptide was conjugated to KLH, it may be conjugated to BSA, or used directly, in a screening assay.)

The foregoing antibodies can be used in methods known in the art relating to the localization and structure of molecules involved in the inflammatory response, including but not limited to the novel carboxylated glycans described herein (e.g., for

Western blotting), measuring levels thereof in appropriate biological samples, etc. For example, the antibodies can be used to detect these carboxylated glycans in a biological sample from an individual. In particular, these carboxylated glycans can be detected from cellular sources, such as, but not limited to, endothelial cells. For example, endothelial cells can be obtained from an individual and lysed (e.g., by freeze-thaw cycling, or treatment with a mild cytolytic detergent such as, but not limited to, TRITON X-100, digitonin, NONIDET P (NP)-40, saponin, and the like, or combinations thereof (See, e.g., International Patent Publication WO 92/08981), or by other methods described herein).

The biological samples can then be tested directly for the presence of carboxylated glycans of interest using an appropriate immunoassay strategy. Alternatively, proteins in the sample can be size separated (e.g., by polyacrylamide gel electrophoresis (PAGE), in the presence or not of sodium dodecyl sulfate (SDS), and the presence of carboxylated glycans of interest is then detected by immunoblotting (Western blotting)).

The foregoing explanations of particular assay systems are presented herein for purposes of illustration only, in fulfillment of the duty to present an enabling disclosure of the invention. It is to be understood that the present invention contemplates a variety of immunochemical assay protocols within its spirit and scope.

### D. Agents Useful in the Invention's Methods

Agents that are useful in the invention's methods include the above-discussed antibodies specific for carboxylated glycans, as, well as antibodies that are specific for polypeptides to which the carboxylated glycans bind. However, other agents and molecules are contemplated to be within the scope of the invention, including, without limitation, naturally occurring compounds and man-made compounds such as those in combinatorial libraries, which can be screened using methods of the invention, and which may be prepared using methods known in the art. These are exemplified by methods for preparing oligonucleotide libraries [Gold et al., U.S. Pat. No. 5,270,163, incorporated by reference]; peptide libraries [Koivunen et al. J. Cell Biol., 124: 373-380 (1994)]; peptidomimetic libraries [Blondelle et al., Trends Anal. Chem. 14:83-92 (1995)] oligosaccharide libraries [York et al., Carb. Res. 285:99-128 (1996); Liang et al., Science 274:1520-1522 (1996); and Ding et al., Adv. Expt. Med. Biol. 376:261-269 (1995)]; lipoprotein libraries [de Kruif et al., FEBS Lett., 399:232-236 (1996)]; glycoprotein or glycolipid libraries [Karaoglu et al., J. Cell Biol. 130:567-577 (1995)]; or chemical libraries containing, for example, drugs or other pharmaceutical agents [Gordon et al., J. Med. Chem. 37:1385-1401 (1994); Ecker and Crook, Bio/Technology 13:351-360 (1995), U.S. Pat. No. 5,760,029, incorporated by reference]. Libraries of diverse molecules also can be obtained from commercial sources.

The invention's agents (such as antibodies) may desirably comprise one or more cytotoxins, therapeutic radionuclides, compounds, chelators and/or imaging reporter molecules as further described below.

i. Cytotoxins and Radionuclides

The agents of the invention may comprise one or more cytotoxins, therapeutic radionuclides, and/or phototherapy compounds. The term “cytotoxin” as used herein refers to any substance having a toxic effect upon cells, including (for example) “tumor chemotherapeutic compounds,” i.e., compounds that delaying the onset of development of tumor development and/or reduce the number, weight, volume, and/or growth rate of tumors. Cytotoxins are exemplified by, without limitation, second messengers such as cAMP; Bacterial toxins such as the exemplary Pertussis toxin, Cholera toxin, and C3 exoenzyme; Lectins such as Ricin A (Engert et al. Blood. 1997 Jan. 15; 89(2):403-10.). Also included are chemotherapeutic agents exemplified by Topisomerase inhibitors such as etoposide, Campothecin irinotecan, topotecan, anthracyclines (doxorubicine, daunorubicine); Microtubule inhibitors such as vincristine, vinblastine, vinorelbine, paclitaxel, docetaxel; Platinum containing compounds such as cisplatin, carboplatin, oxaloplatin, etc.; Alkylating agents such as cyclophosphamide, and ifosfamide; Antimetabolites such as methotrexate and mercaptoprine; Anti-estrogens such as tamoxifen and toremifene; Retinoids such as all trans-retinoic acid; and others such as Adriamycin, gemcitabine, and 5-fluoruracil (Cancer: Principles and Practice of Oncology (1997) Ed. DeVita, Hellman and Rosenberg Lippincott-Raven Publishers, Philadelphia pp 375-498).

Also included within the scope of the invention are cytotoxins such as Maytanisoids (Liu et al. (1996) Proc Natl Acad Sci USA. 93:8618-23.)

The invention's agents may further include therapeutic radionuclides. These are exemplified by Yttrium 90 (Hendrix et al. (2002) J Oncol Nurs. 6:144-8); Bismuth 213 (Sandmaier et al. (2002) Blood. 100:318-26); and Astatine 211 (Kennel et al. (2002) At. Radiation Res. 157:633-41)

The invention's agents may further include phototherapy compounds, such as, without limitation, propenochlorine and benzochlorine (Chen et al. (2002) Cancer J 8:154-63).

ii. Chelators and Imaging Reporter Molecules

The invention's agents may comprise one or more chelators and/or imaging reporter molecules such as those described in U.S. Pat. No. 6,409,990, the entire contents of which are incorporated by reference.

The term “chelator” refers to a molecule that is capable of reacting with another molecule to form a chelate (i.e., cyclic structure) that usually (but not necessarily) contains 5 or 6 atoms in a ring. Preferably, the ring has a central metal ion (such as bivalent copper or bivalent or trivalent iron) that is held in a coordinating complex by one or more groups (as citrate or ethylenediamine) each of which can attach itself to the central ion by at least two bonds. Exemplary chelators include, without limitation, tetraazacyclododecanetetraacetic acid (ROTA) [Sieving et al. (1990) Bioconjugate Chem 1: 65-71], mercaptoacetylglycylglycyl-glycine (MAG3) [Fritzberg et al. (1986) J Nucl Med 27: 111-116], diethylenetriamine pentaacetic acid (DTPA) [Krejcarek et al. (1977) Biochem Biophys Res Comm 77: 581-585], 4-[2-[(2-mercapto-2-methylpropyl)methylamino]-ethyl]-6,6-dimethyl-2-thiomorpholinone (DADT) [Baidoo et al. (1990) Bioconjugate Chem 1: 132-137], and Deferoxamine [Yokoyama et al. (1982) J Nucl Med 23:909-914.].

The terms “imaging reporter,” “imaging reporter molecule,” “imaging molecule,” “reporter” and “reporter molecule” refer to a molecule that is detectable in any detection system, including, but not limited to enzyme (e.g., ELISA, as well as enzyme-based histochemical assays), fluorescent, radioactive, and luminescent systems. Examples of imaging reporters include, without limitation, Technetium-99m (Tc-99m), Indium-111 (In-111), Gallium-68 (Ga-68), Gallium-67 (Ga-67), Rhenium-186 (Re-186) [Visser et al. (1993) J Nucl Med 34: 1953-1963], Rhenium-188 (Re-188) [Guhlke et al. (1998) Nucl Med Biol 25: 621-631]; Iodine-123 (I-123), Iodine-125 (I-125), Iodine-131 (I-131), Iodine (I), Gadolinium (Gd), Ytterbium (Yb) [Krause et al. (1996) Invest Radiol 31:502-511], Dysprosium (Dy) [Vera et al. (2002) Acad Radiol 9:784-792], Europium (Eu), Perflubron-based emulsions [Mattrey et al. (1990) Invest Radiol 25: 915-921], and Microbubble-based emulsions [Sirlin et al. (1999) Ultrasound Med Biol 25: 331-338].

### E. Reducing Extravasation of Leukocytes

The invention also provides methods for reducing extravasation of leukocyte cells in endothelial tissue, comprising: a) providing: i) endothelial tissue comprising leukocyte cells; and an agent that reduces specific binding of a polypeptide to a carboxylated glycan purified by the above-described methods; and b) administering the agent to the endothelial tissue such that specific binding of the polypeptide to the carboxylated glycan is reduced, thereby reducing extravasation of the leukocyte cells in the endothelial tissue. The term “extravasation” refers to efflux of cells across endothelial tissue. Methods for determining extravasation are known in the art (See e.g., Perretti, Gen. Pharm., 31:545 [1998] which reviews the role of annexin I in neutrophil extravasation) and provided herein. For example, Example 10 shows that mesenteric vessels may be histologically examined for intravascular and extravascular accumulation of leukocytes. Also, data herein shows that the carboxylated glycan-specific antibody mABGB3.1 inhibited extravasation of neutrophils and monocytes in a murine model of peritoneal inflammation (Example 34).

In a preferred embodiment, the polypeptide to which the carboxylated glycan specifically binds comprises S100A8, S100A9, S10012, amphoterin, annexin I, or amino acids 1 to 12 of annexin I. In one embodiment, the polypeptide comprises a S100A8•S100A9 heterodimer, (S100A8)₂•S100A9 heterotrimer, or (S100A8)₂•(S100A9)₂ heterotetramer. In another embodiment, the polypeptide comprises S100A12, heteromultimers and/or homomultimers of S100A12 such as multimers containing from 1 to 9, and more preferably from 1 to 6, S100A12 molecules.

With respect to amphoterin, this polypeptide localizes in the cytoplasm of resting cells and lacks a classic secretion signal, and yet it is secreted upon activation (Fages et al., J. Cell Sci., 113:611-620 [2000]; Melloni et al., FEBS Lett., 368:466-470 [1995a]; Rouhiainen et al., Thrombosis Haemostosis 84:1087-1094 [2000]; and Wang et al., supra). Annexin-I, and the S100A8/S100A9 complex, the other two carboxylated glycan-binding lectins, are also cytosolic and secreted by a non-classical pathway (Perretti, Trends Pharmacol. Sci., 18:418-425 [1997]; and Rammes et al., J. Biol. Chem., 272:9496-9502 [1997]), as are the galectins (Hughes, Biochim. Biophys. Acta 1473:172-185 [1999]). The three carboxylate-binding lectins share other properties: all three bind to signal-transducing cell surface receptors (Hori et al., supra; Newton and Hogg, J. Immunol., 160:1427-1435 [1998]; and Walther et al., Mol. Cell., 5:831-840 [2000]) and all have been linked to inflammation (Hofmann et al., Cell 97:889-901 [1999]; Kerkhoff et al., Biochim. Biophys. Acta 1448:200-211 [1998]; and Perretti, supra). Identifying annexin I, S100A8/A9 and amphoterin as a new family of lectins now offers a new dimension to their roles as effectors in signaling pathways.

In addition, lectins that mediate the glycan-based interactions with the endothelium were of interest. During the development of the present invention, affinity columns containing immobilized carboxylated glycans were found to bind three proteins from solubilized bovine lung in a carboxylate dependent manner. As discussed in greater detail herein, two of these proteins are the inflammation-related proteins annexin I and S100A8/A9 complex. The third lectin was identified as amphoterin, a protein linked to neuronal development and invasive cell migration (Hori et al., J. Biol. Chem., 270:25752-25761 [1995]; Rauvala et al., Matrix Biol., 19:377-387 [2000]; and Rauvala and Pihlaskari, J. Biol. Chem., 262:16625-16635 [1987]), differentiation of erythroleukemia cells (Melloni et al., Biochem. Biophys. Res. Commun., 210:82-90 [1995b]), endotoxemia (Abraham et al., J. Immunol., 165:2950-2954 [2000]; and Wang et al., Science 285:248-251 [1999]), and tumor growth and metastasis (Taguchi et al., Nature 405:354-360 [2000]).

As indicated above, the two leukocyte calcium-binding proteins that bound in a carboxylate-dependent manner were identified as S100A8 and annexin I. An intact N-terminus of annexin I and heteromeric assembly of S100A8 with S100A9 (i.e., another member of the S100 family) was found to be necessary for this interaction. The novel glycans structure recognized by these neutrophil proteins is highly enriched on endothelial cell surfaces in various mammals (e.g., humans, mice, and bovines). In addition, it is contemplated that this epitope is involved in inflammatory processes. Thus, the present invention provides compositions involved in a novel pathway involving inflammation that is different from previously described systems involving integrins and selectins. The present invention further provides methods to utilize these compositions and novel pathway in the development of additional compositions and methods suitable for altering the inflammatory pathway, as well as means to further elucidate the inflammatory response. Indeed, it is contemplated that the present invention will find use in the development of approaches to block acute inflammation by affecting neutrophil function.

The present invention also provides purified human S100A8/A9 complexes and recombinant human annexin I. These complexes and recombinant annexin I demonstrate carboxylate-dependent binding to immobilized bovine lung carboxylated glycans and recognize a subset of mannose-labeled endothelial glycoproteins immunoprecipitated by mAbGB3.1. Saturable binding of S100A8/A9 complex to endothelial cells is also blocked by mAbGB3.1. Thus, the present invention provides carboxylated glycans that are important in leukocyte trafficking due to their interactions with proteins known to modulate extravasation. In addition, the carboxylated glycans provide a 10-fold improvement in the specific binding of amphoterin and a cell-surface signalling molecule called RAGE (Receptor for Advanced Glycation Endproducts). The amphoterin-RAGE interaction is a key determinant in cancer cell growth, metastasis, and metalloproteinase activation.

The two migration inhibitory factor related proteins S100A8 and S100A9 belong to the large family of S100 proteins whose members have the EF hand calcium binding structures (See e.g., Teigelkamp et al., J. Biol. Chem., 266:13462 [1991], for a recent review). These heteromeric complexes are found in sera of patients with cystic fibrosis, chronic bronchitis, and rheumatoid arthritis, thereby indicating extracellular roles for these complexes (19-van Heyningen et al., Nature 315:513 [1985]; Roth et al., Immunobiol., 186:304 919920; and Brun et al., J. Rheumatol., 21:733 [1994]). Prior to the development of the present invention, well-defined functions of S100A8 and S100A9 either as individual proteins or as complexes have been elusive. Existing evidence suggested that they may be involved in macrophage differentiation and inflammation. When monocytes extravasate through the endothelium and become resident macrophages, these antigens are lost (Zwadlo et al., Clin. Exp. Immunol., 72:510 [1988]). However, at sites of chronic inflammation in patients with rheumatoid arthritis, sarcoidosis, and tuberculosis, tissue macrophages continue to express S100A8/A9 (Zwadlo et al., supra; and Odink et al., Nature 330:80 [1987]). These proteins have also been found coating vascular endothelium adjacent to marginating neutrophils and monocytes, suggesting that secretion of S100A8/A9 on to vessel walls is involved in the migration of leukocytes into tissues (Hogg et al., Eur. J. Immunol., 19:1053 [1989]). In addition, Kerkhoff et al report that only monocytes expressing the S100A8/A9 complex preferentially migrate through the microvascular endothelium (Kerkoff et al., Pathobiol., 67:230 [1999]).

Although an understanding of the mechanism is not necessary in order to utilize the present invention, it is not clearly understood how the S100 proteins mediate these interactions with the endothelium. Recent studies show that S100A8/A9 binds to unsaturated fatty acids with high affinity, and may be involved in arachidonic acid metabolism (Siegenthaler et al., J. Biol. Chem., 272:9371 [1997]; Klempt et al., FEBS Lett., 408:81 [1997]; and Kerkoff et al., J. Biol. Chem., 274:32672 [1999]). Cell surface S100A8/A9 binding sites have also been detected on human leukemia cell lines (Koike et al., J. Biochem., 123:1079 [1998]). S100A9 has also been shown to modulate adhesion of neutrophils to fibrinogen via β2 integrin Mac1, by recognizing a distinct but uncharacterized pertussis toxin-sensitive G-protein coupled receptor on neutrophils (Newton and Hogg, J. Immunol., 160:1427 [1998]).

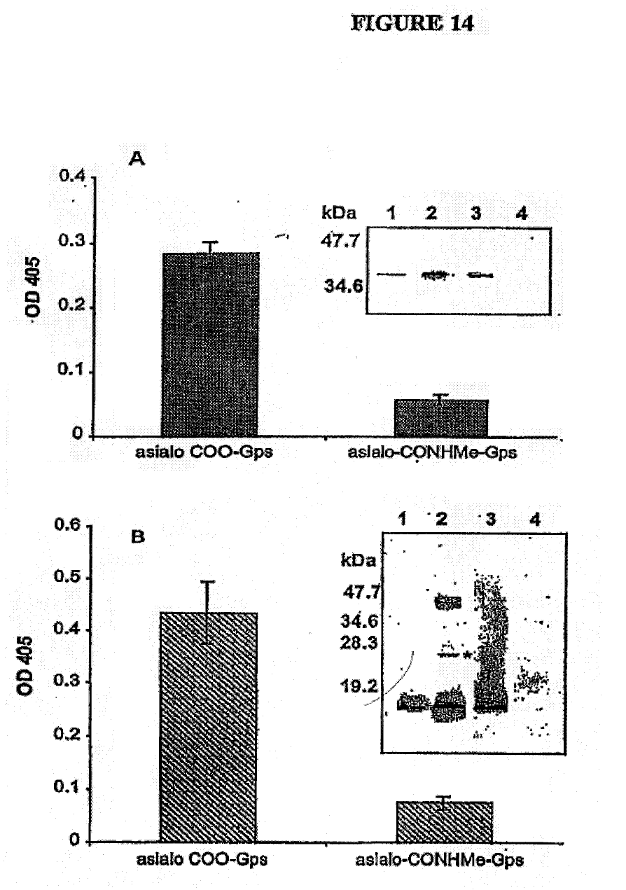

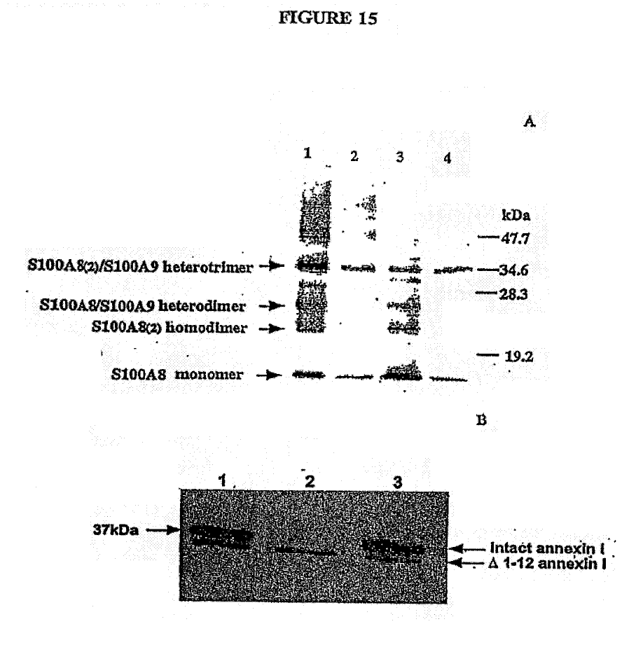

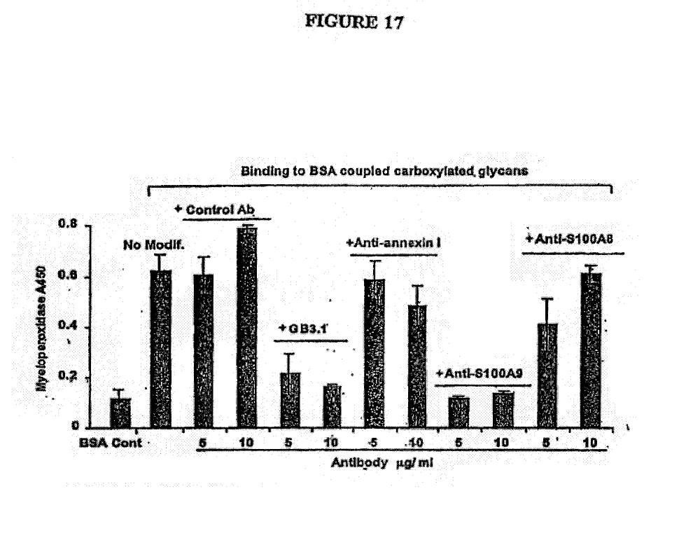

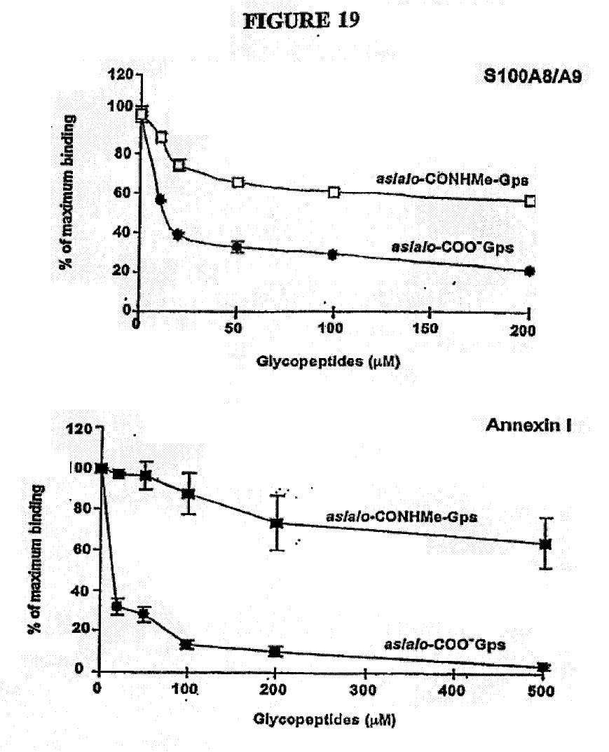

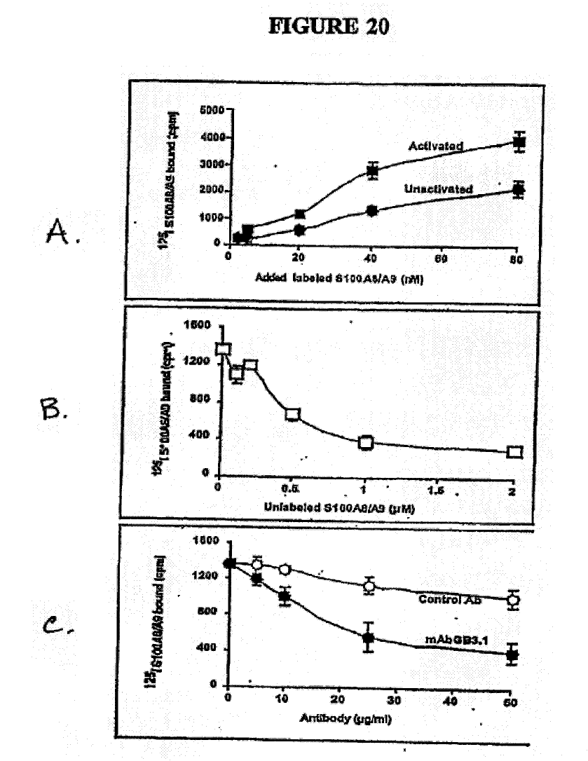

Based on data obtained during the development of the present invention, there are several lines of evidence that S100A8/A9, and especially the S100A9 component, function by directly interacting with novel carboxylated glycans on endothelial cells. First, heteromeric complexes of S100A8/A9 are depleted from neutrophil extracts after binding to the glycans, and this binding is carboxylate-dependent (See, FIG. 14, Panel B and FIG. 15). As shown in FIG. 17, adhesion of neutrophils to the immobilized glycans is effectively inhibited by both anti-S100A9 and mAbGB3.1. Also, as shown in FIG. 19, purified S100A8/A9 complex I binds to the novel glycans in a carboxylate-dependent manner. In addition, S100A8/A9 complex cross-reacts with mannose-labeled glycoproteins immunoprecipitated by mAbGB3.1 (See, Table 3). Finally, specific binding of S100A8/A9 to endothelial cell surface is blocked by mAbGB3.1 (FIG. 20). Recombinant S100A8 alone also does not show specific binding to the glycans (data not shown), indicating that heteromeric assembly of the S100 proteins was necessary for this interaction. The binding also appears to be exclusive to S100A8/A9, since binding of two other S100 proteins S100A1 and S100B remained constant between carboxylated and neutralized glycans under these conditions (data not shown).

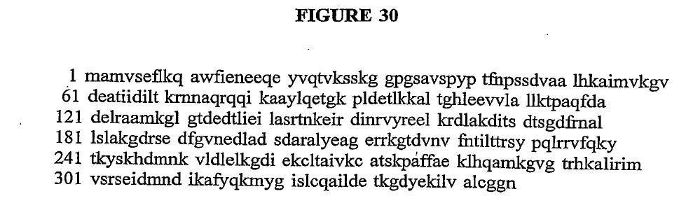

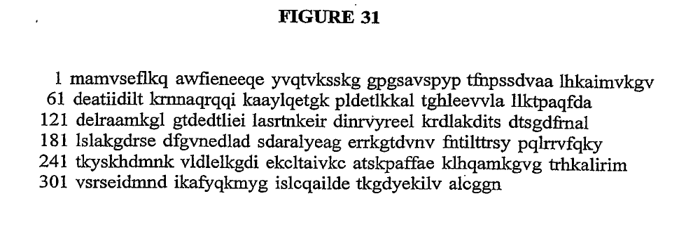

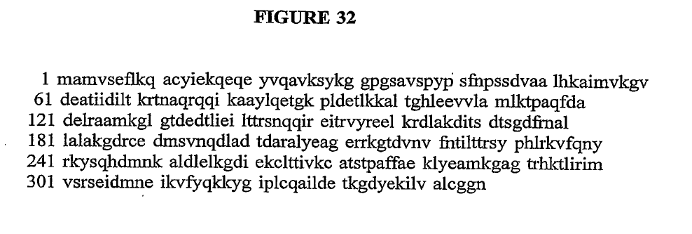

The term “annexin I,” also known as “lipocortin I,” refers to polypeptide that belongs to a family of 13 distinct calcium binding proteins characterized by a common C-terminal core structure consisting of 4 or 8 highly conserved repeating motifs, which bind calcium and phospholipids, and a variable N-terminus (Raynal and Pollard, Biochem. Biophys. Acta 1197:63 [1994]). Annexin I is distributed in different tissues, but is abundant in cells of the hematopoietic lineage, particularly granulocytes and monocytes, where it makes up as much as 2-4% of total cytosolic protein (Perretti, Trends Pharm. Sci., 18:418 [1997]). Annexin I protein has an apparent relative molecular mass of 40 kDa, with phospholipase A2 inhibitory activity. This protein contains four homologous repeats. Each contains an ‘endonexin fold,’ a consensus sequence common to all annexins. A pair of these repeats may form one binding site for calcium and phospholipid. Annexin I is exemplified by human annexin I shown in FIG. 30 (GenBank No. NP_{—}000691) [Walther et al. (2000) Mol. Cell 5 (5), 831-840; Kamal et al. (2001) Clin. Exp. Allergy 31 (7), 1116-1125] and FIG. 31 (GenBank No. LUHU) [Arcone et al. (1993) Eur. J. Biochem. 211 (1-2), 347-355], and also by rat annexin I shown in FIG. 32 (GenBank No. LURT1) [Hyatt et al. (1994) Biochemistry 33 (5), 1223-1228].

The annexin I present in the bovine lung extract could have originated either from the lung tissue or from blood cells or both. The lung also contains annexins II and V (Flower and Rothwell, Trends Pharmacol. Sci., 15:71 [1994]), but repeated isolation of annexin I alone on the columns used during the development of the present invention showed that its binding was specific. It is contemplated that the specificity of binding is imparted by the biologically active N-terminus (˜40 amino acids in length), which is unique for each member of the annexin family (Raynal and Pollard, Biochem. Biophys. Acta 1197:63 [1994]). The N-terminal alanine of intact annexin I is acetylated and resistant to Edman degradation (Hall et al., Proc. Natl. Acad. Sci. USA 90:1927 [1993]); the presence of the Δ1-12 annexin I enabled identification of the sequence, though the intact form was also isolated (as found in immunoblots; data not shown). The S100A8/A9 complex on the other hand is present exclusively in leukocytes (Edgeworth et al., J. Biol. Chem., 266:7706 [1991]; and Schafer et al., Trends Biochem. Sci., 21:134 [1996]), and therefore likely originated from the trapped blood or sequestered neutrophils in bovine lung. During the development of the present invention, it was also found that human homologues of annexin I and S100A8 from neutrophil lysates were bound to the glycopeptide columns and immobilized glycans in a carboxylate-dependent manner (See, FIG. 14).

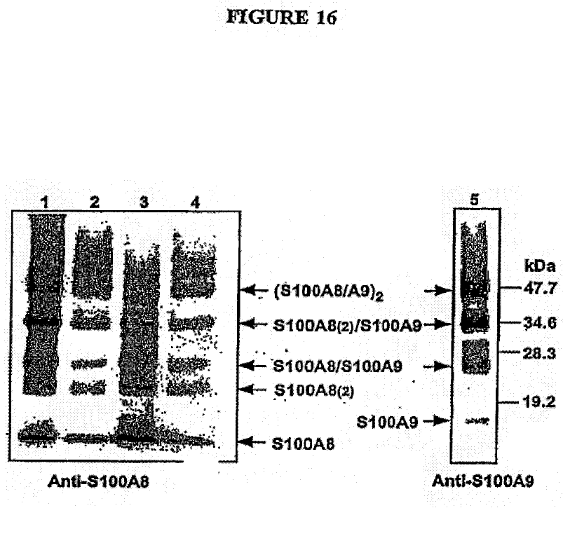

A growing body of evidence from studies on inflammation in animal models and humans suggests that annexin I potently inhibits neutrophil extravasation (See e.g., Perretti, Gen. Pharm., 31:545 [1998] for a review). Annexin I is also strongly induced by glucocorticoids and is considered to mediate some of the well known anti-inflammatory effects of these hormones (Goulding et al., Inflamm. Res., 47 (suppl. 30:S158 [1998]). The N-terminus of annexin I has been shown to play an important modulatory role in the biological effects of the protein. For example, several studies have shown that the N-terminal peptide Ac 2-26 mimics the potent anti-inflammatory effects of intact annexin I (Perretti, Gen. Pharm., 31:545 [1997]). The amino terminal domain regulates interactions with membranes (Wang and Cruetz, Biochem., 33:275 [1994]), and is directly involved in binding to S100 proteins (Mailliard et al., J. Biol. Chem., 271:719 [1996]; and Rety et al., Structure 8:175 [2000]). Most notably, the N-terminal peptide Ac2-12, which as shown herein, appears essential for binding to the novel glycans (See, FIG. 16), and has been shown to be as effective in detaching adherent neutrophils from murine postcapillary venules as the intact protein (Lim et al., Proc. Natl. Acad. Sci. USA 95:14535 [1998]). An endogenous protease catalyzes cleavage at tryptophan 12 of intact annexin I in a Ca²⁺ dependent manner (Chuah and Pallen, J. Biol. Chem., 264:21160 [1989]), and proteolysis is believed to regulate the action of externalized annexin I (Goulding et al., Inflamm. Res., 47 (suppl. 3):S158 [1998]). The mechanisms by which annexin I and its peptide mimetics affect neutrophil extravasation have remained elusive so far. However, an understanding of these mechanisms is not necessary in order to use the present invention. Annexin I binding sites on neutrophils and monocytes have been identified, and the protein has recently been shown to be a ligand for the formyl peptide receptor (See, Euzger et al., Mediators Inflamm., 8:53 [1999]; and Walther et al., Mol. Cell., 5:831 [2000]). Annexin I has also been reported to regulate the MAPK/ERK signaling pathway (Allridge et al., J. Biol. Chem:, 274:37620 [1999]).

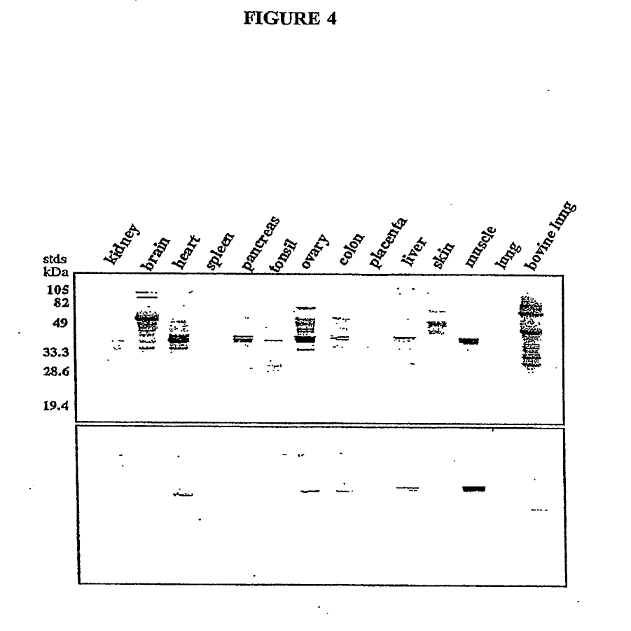

Based on data obtained during the development of the present invention, it is contemplated that at least one of annexin I's functions involved the interaction with novel carboxylated glycans. Annexin I from human neutrophil lysates shows carboxylate dependent binding to the glycans (See, FIG. 4, Panel A). In addition, depletion experiments indicate that the N-terminal 1-12 amino acids of annexin I may be important for optimal binding (See, FIG. 16). Recombinant full-length human annexin I binds to the novel glycans in a carboxylate dependent manner (See, FIG. 19).

However, it is contemplated that in some embodiments of the present invention, the precise epitopes recognized by annexin I and S100A8/A9, and the physiological processes mediated in vivo by annexin I and S100A8/A9 differ. For example, annexin I precipitates only about 2-3% of mannose-labeled glycoproteins from endothelial cells compared to 9% by S100A8/A9 (data not shown), indicating that some recognition epitopes preferred by annexin I are on O-glycans. Also, unlike S100 proteins, annexin I does not modulate neutrophil adhesion to endothelial monolayers, but impedes neutrophil emigration (See e.g., Perretti et al., Nature Med., 2:1259 [1996]). In addition, it has been recently shown that annexin I inhibits monocyte adhesion to microvascular endothelial cells by means involving its N-terminal domain (See, Solito et al., J. Immunol., 165:1573 [2000]). This may explain the increased adhesion of leukocytes to endothelial cells in the presence of mAbGB3.1 after induction of peritonitis in mice, as described herein.

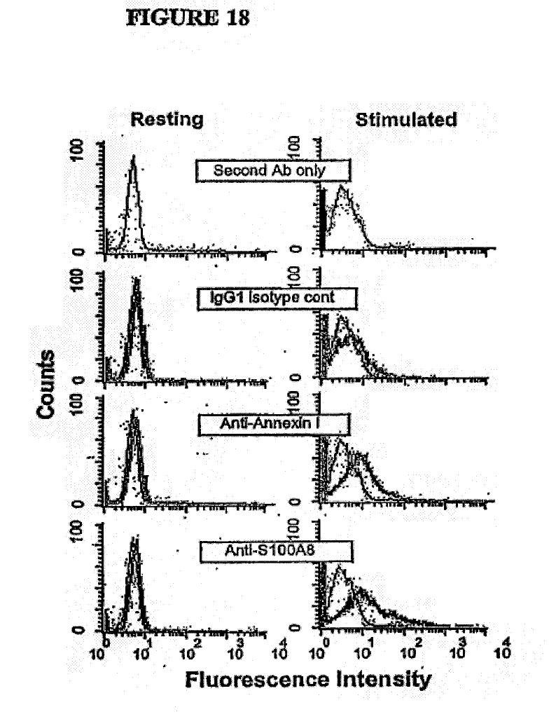

As indicated above, neutrophils acquire carboxylate-dependent binding sites for the novel glycans after short term activation. The novel glycans also bind to secretions from activated neutrophils (data not shown). Annexin I and S100 proteins are cytosolic and lack classical leader peptide sequences and transmembrane domains. During the development of the present invention, it was found that some S100A8/A9 complexes are present in the neutrophil membranes and in secretions within minutes after activation (See, FIG. 18). Upon calcium binding they are known to translocate from cytoplasm to cytoskeleton and plasma membrane (Roth et al., Blood 82:1875 [1993]; and Mahnke et al., J. Leukoc. Biol., 57:63 [1995]). In addition, neutrophil activation with opsonized zymosan also correlates with translocation of S100A8 to the plasma membrane. Secretion of the S100 proteins is believed to be tubulin, energy, and protein kinase C dependent (Rammes et al., J. Biol. Chem., 272:9496 [1997]). Also, following neutrophil adhesion to endothelial monolayers, endogenous annexin I has been detected on the surface of adherent leukocytes, in an EDTA elutable-pool (Perretti et al., Nature Med., 2:1259 [1996]). These findings conform with the characteristics of annexin I and S100A8 and S100A9 as a class of proteins which are secreted by a novel non-Golgi pathway (Meusch et al., Trends Biochem. Sci., 15:86 [1990]; and Hughes, Biochim. Biophys. Acta 1473:172 [1999]). This class of proteins also includes growth factors and the S-type lectins or galectins.

The data presented herein clearly indicate that the novel carboxylated endothelial glycans bind to soluble leukocyte proteins which are secreted upon activation, and which are known to mediate inflammatory responses. Based upon the data herein, it is contemplated that a novel pathway in acute inflammation is provided herein. It is further contemplated that endothelial glycoproteins carry the novel carboxylated glycans. In addition, it is contemplated that binding partners involved in mediating the functions of S100A8/A9 and annexin I are involved. Furthermore, it is contemplated that signaling mechanisms by which these proteins bring about their inflammatory responses in vivo are involved.

### F. Reducing Adherence of Leukocyte Cells to Endothelial Cells

The invention further provides methods for reducing adherence of leukocyte cells to endothelial cells, comprising: a) providing: i) leukocyte cells; ii) endothelial cells; and an agent that reduces specific binding of a polypeptide to a carboxylated glycan purified by the methods described above; and b) contacting the leukocyte cells, the endothelial cells, and the agent such that adherence of the leukocyte cells to the endothelial cells is reduced in the presence of the agent compared to in the absence of the agent.

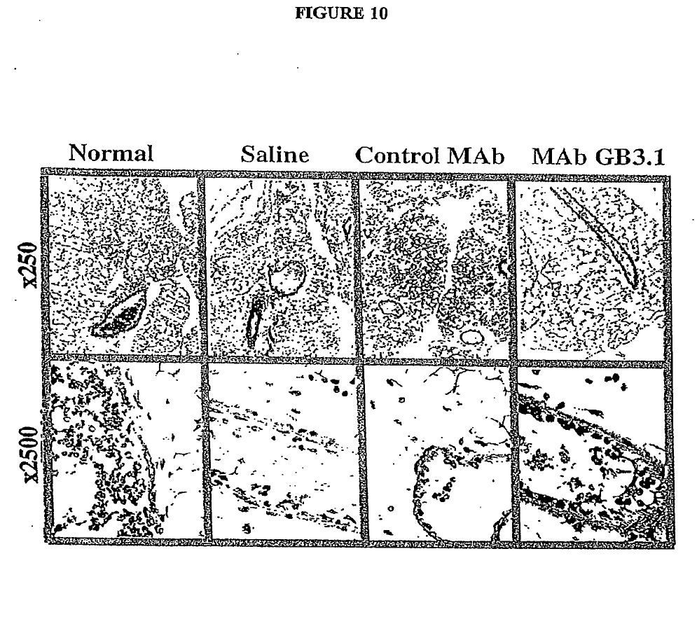

The term “adherence” refers to attachment as determined using art known methods as well as methods disclosed herein. The term ““reduced adherence”refers to a quantity of adherence that decreased by an amount that is statistically significant as determined using any art-accepted statistical method of analysis. Preferably the quantity of adherence is reduced by at least 10%, more preferably by at least 50%, yet more preferably by at least 75%, even more preferably by at least 90%. In a most preferred embodiment, the quantity of adherence is undetectable. For example, FIG. 10 shows that mice treated with mAbGB3.1 showed decreased adherence of leukocytes to mesenteric venules compared to mice treated with saline or control antibody.

### G. Reducing Inflammation and Cancer

The present invention further provides means to identify and modulate binding of various ligands involved in pathological processes. For example, mammalian lectins bind to a variety of glycans and mediate important biological functions. Many lectin-glycan interactions involve anionic glycans (Bernfield et al., Ann. Rev. Biochem., 68:729-777 [1999]; Crocker et al., Glycobiol., 8:v [1998]; Fukuda et al., J. Cell. Biol., 147:467-470 [1999]; Kornfeld, Biochem. Soc. Trans., 18:367-374 [1990]; Nakayama et al., Pathol. Int., 48:665-677 [1998]; Springer, Ann. Rev. Physiol., 57:827-872 [1995]; and Varki, Proc. Natl. Acad. Sci. USA 91:7390-7397 [1994]). As indicated above, during the development of the present invention, non-selectin ligand carboxylated endothelial cell glycans that mediate leukocyte-endothelium interactions during inflammation were identified.

In particular, the invention provides a method for reducing inflammation in a tissue in a mammalian subject, comprising: a) providing: i) a tissue; and an agent that reduces specific binding of a polypeptide to a carboxylated glycan purified by the methods discussed above; and b) administering the agent to the tissue such that inflammation in the tissue is reduced in the presence of the agent compared to in the absence of the agent. Also provided herein is a method for reducing cancer in a subject, comprising: a) providing: i) a subject; and ii) an agent that reduces specific binding of a polypeptide to a carboxylated glycan purified by the above methods; and b) administering the agent to the subject such that cancer in the subject is reduced in the presence of the agent compared to in the absence of the agent.

The term “reducing” when in reference to a disease (such as inflammation or cancer) means diminish, delay, or eliminate (objectively and/or subjectively) the level of one or more undesirable symptoms that are associated with the disease. As used herein, the term “diminishing” symptoms refers to decreasing the levels of one or more symptoms. The term “delaying” symptoms refers to increasing the time period between exposure to the immunogen and the onset of one or more symptoms. The term “eliminating” symptoms refers to completely “reducing” and/or completely “delaying” one or more symptoms.

The term “cancer” refers to a tissue growth that contains a cancer cell. Cancer includes benign cancer as well as malignant cancer. Benign cancer is exemplified, but not limited to, a hemangioma, glioma, teratoma, and the like. Malignant cancer include, for example, a carcinoma, sarcoma, glioblastoma, astrocytoma, neuroblastoma, retinoblastoma, and the like. A “cancer cell” refers to a cell undergoing early, intermediate or advanced stages of multi-step neoplastic progression. A cell in the early stages of malignant progression is referred to as “hyperplastic cell” and is characterized by dividing without control and/or at a greater rate than a normal cell of the same cell type in the same tissue. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate one or more secondary cancers, i.e., “metastases.” The term “cancer” as used herein includes a malignant neoplasm, which may or may not be metastatic. Malignant neoplasms include, for example, carcinomas such as lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

### Experimental

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); BSA (bovine serum albumin); CFA (complete Freund's adjuvant); IFA (incomplete Freund's adjuvant); IgG (immunoglobulin G); IM (intramuscular); IP (intraperitoneal); IV (intravenous or intravascular); SC (subcutaneous); H₂O (water); HCl (hydrochloric acid); aa (amino acid); by (base pair); kb (kilobase pair); kD (kilodaltons); gm, G, and g (grams); (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); mAb (monoclonal antibody); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); SDS-PAGE (sodium dodecyl sulfate polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); PMSF (phenylmethylsulfonyl fluoride); RT-PCR (reverse transcription PCR); SDS (sodium dodecyl sulfate); Tris (tris(hydroxymethyl)aminomethane); w/v (weight to volume); v/v (volume to volume); BAP (biotinylated diaminopyride); BSA (bovine albumin); DEAE (diethyl aminoethyl); BCIP/NBT (5-bromo-4-chloro-3-indolyl phosphate/nitroblue tetrazolium); BS³ (bis-sulfosuccinimidyl suberate); CPAE (calf pulmonary artery endothelial cells); EDC (1-ethyl-3(3-dimethylaminopropyl) carodiimide); EDTA (ethylenediamine tetraacetic acid); DTT (dithiothreitol); NHS (N-hydroxy-succinimide); PBS (phosphate buffered saline); HBSS (Hank's balanced salt solution); MEM (minimal essential medium); HUVEC (human umbilical vein endothelial cells); CHO Lec2 (Chinese hamster ovary cell glycosylation mutants defective in CMP-Sia transporter); PAF (platelet activating factor); PMA (phorbol myristate acetate); GAG (glycosaminoglycan); PNGase F and PNGase A (peptide-N-glycosidases F and A, respectively); EGF (epidermal growth factor); HPAEC-PAD (high pH anion-exchange chromatography-pulse amperometric detection); MAPG TNFα (tumor necrosis factor α); ERK (extracellular signal-related kinase); MAPK (mitogen-activated protein kinase); MRP8 and MRP9 (migration inhibitory related proteins 8 and 9); ESI-MS (electrospray ionization mass specrometry); MADI-TOF (matrix-assisted laser desorption ionization time-of-flight); ELISA (enzyme-linked immunosorbent assay; also EIA); HMGB1 (high mobility group box 1); HNK-1 epitope (3-sulfoglucuronyl β1-3 galactoside; originally identified in human NK cells); JNK (c-jun N terminal kinase); MMP (matrix metalloproteinase); QAE (quartemary aminoethyl); RAGE (receptor for advanced glycation end products); sRAGE (soluble RAGE); Amersham (Amersham Pharmacia Biotech, Arlington Heights, Ill.); ICN (ICN Biomedicals, Inc., Aurora, Ohio); Nycomed (Nycomed Pharma, Oslo, Norway); PharMingen (PharMingen, La Jolla, Calif.); PelFreeze (PelFreeze Biologicals, Rogers, Ark.); Cell Application System (Cell Application Systems, San Diego, Calif.); ATCC (American Type Culture Collection, Rockville, Md.); BioRad (BioRad, Richmond, Calif.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); Invitrogen (Invitrogen Corp., San Diego, Calif. and Groningen, the Netherlands); Qiagen (Qiagen, Hilden, Germany); Kodak (Eastman Kodak Co., New Haven, Conn.); New England Biolabs (New England Biolabs, Inc., Beverly, Mass.); Novagen (Novagen, Inc., Madison, Wis.); Pharmacia (Pharmacia, Inc., Piscataway, N.J.); Nunc (NUNC, Naperville, Ill.); Sigma (Sigma Chemical Co., St. Louis, Mo.); Sorvall (Sorvall Instruments, a subsidiary of DuPont Co., Biotechnology Systems, Wilmington, Del.); Stratagene (Stratagene Cloning Systems, La Jolla, Calif.); Whatman (Whatman LabSales, Hillsboro, Oreg.); Calbiochem (Calbiochem, La Jolla, Calif.); Boehringer-Mannheim (Boehringer Mannheim, Indianapolis, Ind.); Promega (Promega Corp., Madison, Wis.); Pierce (Pierce Chemical Company, Rockford, Ill.); Binding Site (The Binding Site, San Diego, Calif.); Xenopore (Xenopore Corporation, Hawthorne, N.J.); Molecular Probes (Molecular Probes, Eugene, Oreg.); American Radiolabeled Chemicals (American Radiolabeled Chemicals, St. Louis, Mo.); TosoHaas (TosoHaas, Montgomeryville, Pa.); Biogenesis (Biogenesis, Inc., Sandown, N.H.); Bachem (Bachem Bioscience Inc., King of Prussia, Pa.); Genset (Genset Corporation, La Jolla, Calif.); Applied Biosystems (Applied Biosystems, Foster City, Calif.); Perseptive Biosystems (PerSeptive Biosystems, Framingham, Mass.); Dako (Dako Corp., Carpinteria, Calif.); Harland (Harland Sprague Dawley, Inc., Indianapolis, Ind.); and Cell Applications (Cell Applications, San Diego, Calif.).

The following materials were from the sources indicated:

Monoclonal antibody mAbGB3.1 directed against the novel carboxylated glycan was generated as described in Example 3. CPAE cells were purchased from ATCC, and HUVECs were from Cell Applications. The BAP used in these experiments was prepared as known in the art (See e.g., Rothenbert et al., Proc. Natl. Acad. Sci. USA 90:11939 [1993]; and Toomre and Varki, Glycobiol., 4:653 [199]). Recombinant streptavidin, bovine lung acetone powder, glutaraldehyde, immunoglobulin isotyping kit, Protein-G Sepharose, platelet activating factor (PAF), phorbol myristate acetate (PMA), TNF-α, anti-human PECAM (CD31), monoclonal anti-BSA, R-phycoerythrin conjugated anti mouse IgG, exoglycosidases and zymosan were obtained from Sigma. Ionomycin was obtained from Calbiochem, while the PNGase F was obtained from New England Biolabs, and the PNGase A was a kind gift of Seikagaku America, Falmouth, Mass. Proteinase K was obtained from Boehringer-Mannheim; while the Biogel P2 and peroxidase linked goat anti-mouse IgG were obtained from Bio-Rad, and the 1-ethyl-3[3-dimethylaminopropyl]carbodiimide hydrochloride (EDC) and N-hydroxy-succinimide (NHS), were obtained from Pierce. Alkaline phosphatase conjugated goat anti-mouse IgG was obtained from Promega, the peroxidase linked streptavidin was obtained from the Binding Site, and the Cy3 monoclonal antibody labeling kit was obtained from Amersham. The Monopoly Resolving Medium was obtained from ICN Biomedicals, while nycoprep was obtained from Nycomed Pharma, FITC anti mouse Ly-6G (Gr-1) and PE anti-mouse CD11b (Mac-1) were obtained from PharMingen, and covalent binding plates were obtained from Xenopore. Bovine tissues were obtained from PelFreeze, while human tissue sections were obtained from the Histology Core Facility of the Cancer Center, University of California, San Diego. The human. umbilical vein endothelial cells (HUVECs) were obtained from Cell Applications System, and calf pulmonary artery endothelial cells (CPAE) were obtained from ATCC. The fMLP and PAF were obtained from Sigma; DEAE Sephadex A25 and Sephadex G25 were obtained from Pharmacia, while the Affigel-10 was obtained from BioRad. The NaI¹²⁵ was obtained from American Radiolabeled Chemicals, while the iodobeads iodination reagent, GelCode Blue, and BS³ were obtained from Pierce. The Phenyl-650C Toyopearl Resin was obtained from TosoHaas. The mouse anti-bovine annexin I was obtained from Biogenesis, while the mouse anti-human S100A8 and S100A9 antibodies were obtained from Bachem. The annexin I cDNA (ATCC number 65115) was obtained from ATCC, while the expression vector pET23b was obtained from Novagen, restriction enzymes and T4 DNA ligase were obtained from Promega, and the nucleotide primers were obtained from Genset. The Western blotting chemiluminescence detection reagent kit was obtained from Amersham Pharmacia; and the [2-³]mannose was obtained from American Radiolabeled Chemicals.

The following chemicals and reagents used in assessment of RAGE, amphoterin binding and neurite growth were obtained as indicated below. Expression vector pQE-32, E. coli strain M15p(REP4), Ni-NTA-resin and anti-penta-His mAb, were obtained from Qiagen; and the vector pIZ/V5-His was obtained from Invitrogen. RAGE cDNA fragment and sRAGE were generous gifts from Dr. Angelika Bierhaus, University of Tubingen and Dr. Aim-Marie Schmidt, Columbia University, respectively. N18 mouse neuroblastoma cells stably transfected with full length or cytoplasmic domain-deleted RAGE were generated as known in the art (Huttunen et al., J. Biol. Chem., 275:40096-40105 [1999]). Preparation of GAG-free, carboxylate-enriched bovine lung glycopeptides, and generation of anti-carboxylate monoclonal antibody mAbGB3.1 were as described. Purification of baculovirus expressed rat amphoterin and generation of affinity purified rabbit antibodies to amphoterin were conducted as known in the art (See, Parkkinen et al., J. Biol. Chem., 268:19726-19738 [1993]). Anti-I-INK-1 antibody and culture supernatants containing anti-HNK-1 reactive proteins from Lec2 cells transfected with 5 glucuronyl transferase and HNK-1 sulfotransferase (See, Ong et al., J. Biol. Chem., 273:5190-5195 [1998]) were kindly provided by Drs. Junya Mitoma and Minoru Fukuda, Burnham Institute, La Jolla, Calif. Human tumor cell lines were generously provided by St. William Stallcup, Burnham Institute, La Jolla, Calif. Fresh bovine lung tissues were obtained from Mory's Meats, Escondido, Calif.

### Example 1

### Preparation of Neoglycoproteins for Immunization and Screening

Early in the development of the present invention, it was determined that biotinylated diaminopyridine (BAP) conjugated glycans presented in multivalent arrays on streptavidin can evoke an IgG serum immune response in mice (Rothenberg et al., Proc. Natl. Acad. Sci. USA 90:11939 [1993]; and Toomre and Varki, Glycobiol., 4:653 [1994]). As discussed in more detail below, to generate antibodies directed against the novel carboxylate-associated negative charge, a mixture of anionic hydrazine-released bovine lung glycans were coupled to BAP.

In these experiments, anionic bovine lung glycans of moderate negative charge, coupled to BAP (as described in U.S. Pat. No. 5,449,781 to Varki et al., the entirety of which is hereby incorporated by reference) were treated with Arthrobacter ureafaciens sialidase (10 mU), jack bean β-N-acetylhexosaminidase (53 mU), bovine testicular β-galactosidase (2 mU), coffee-bean α-galactosidase (5 mU) and jack bean α-mannosidase (2-5 mU) in ˜20 μl. of sodium citrate buffer (pH=4.5). These multiple exoglycosidases were inactivated by heating at 100° C. for 5-10 min, and the sample was fractionated on a DEAE-2SW HPLC column as known in the art (See e.g., Toomre and Varki, Glycobiol., 4:653-663 [1994]). Thus, to enrich for coupled glycans bearing the novel carboxylate, multiple exoglycosidase treatment to molecules with >2 negative charges, that had been obtained by anion exchange chromatography on DEAE-HPLC were applied. The exoglycosidases chosen were highly specific and did not affect the negative charge of the carboxylate (data not shown).

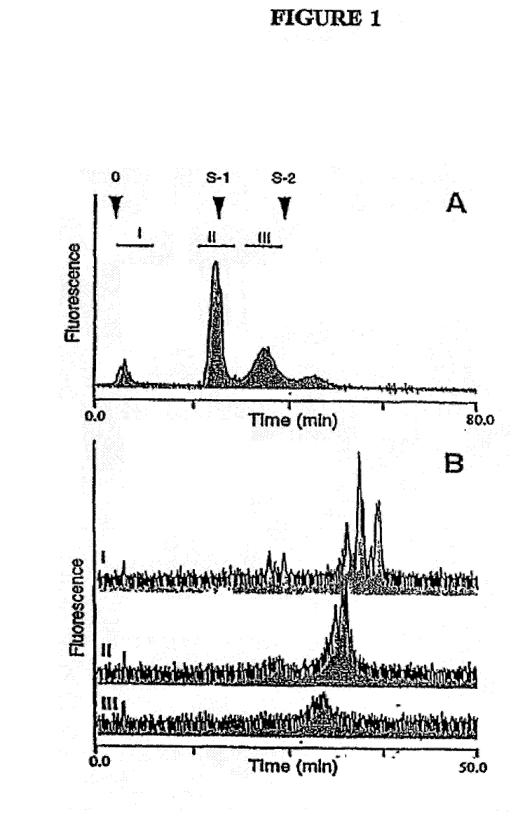

Specific subfractions from the DEAE column were also analyzed by reverse-phase HPLC as known in the art (See, Toomre and Varki, supra). The mixed BAP coupled oligosaccharides enriched for the carboxylate residue (See, FIG. 1) were mixed with streptavidin in phosphate buffered saline (PBS) in the ratio of 1:3 and the mixture was kept at 4° C. for 1 hour prior to immunization of mice. To generate BSA neoglycoproteins for screening, glycopeptides were prepared from bovine lung acetone powder by Proteinase K digestion followed by purification on a Biogel P2 column run in 0.1 M ammonium formate, pH 6.5. Glycopeptides eluting in the void volume were conjugated to BSA using either glutaraldehyde or EDC following standard protocols known in the art.

Thus, following the mixed exoglycosidase treatment, the fractions that shifted to an elution position on DEAE-HPLC that is typical for glycans with a single negative charge were collected (FIG. 1, Panel A, Pool II). Because sialidase was included in the digestions, the mixture was expected to be enriched for molecules carrying one copy each of the novel carboxylate modification. Indeed, this material eluted from reverse-phase HPLC (FIG. 1, Panel B) in the general region expected for BAP-coupled N-glycans with one or two negative charges, such as mono or bisialylated biantennary N-glycans. This mixture of BAP-coupled glycans was bound to streptavidin to form multivalent pseudoneoglycoproteins.

The BSA conjugates (herein referred to as “BSA neoglycoproteins” for the glutaraldehyde-coupled glycopeptides, unless otherwise stated) were analyzed by PAGE gels and by neutral sugar estimation (phenol sulfuric acid assay), which indicated an average of 2.5 moles of N-glycans coupled per mol of protein (assuming a typical biantennary N-glycan, data not shown).

Injection of these conjugates into mice elicited a serum IgG response that was at least partly directed against the oligosaccharides, as detected by reactivity in ELISA with BSA coupled bovine lung glycopeptides or whole bovine lung homogenates (data not shown).

### Example 2

### Modification of Carboxylate Groups by Carbodiimide Activation and Reaction with Methylamine

Glycopeptides generated from bovine lung acetone powder as described above were desialylated by mild acid treatment (10 mM HCl, 30 min at 100° C.) and lyophilized. (herein, these glycopeptides are referred to as “asialo-COO⁻ glycopeptides”). Then, 500 nmoles of such glycopeptides (by neutral sugar estimation) were dissolved in 50 μl of 50 mM MES buffer, pH 5.5, followed by addition of 100 μl of 1 M methylamine. Then, 50 μl of EDC/NHS from a freshly prepared stock solution of 100 mg EDC and 50 mg NHS/ml water were added, and the mixture was incubated at 37° C. After 1 h, another 50 μl of fresh EDC/NHS was added and the incubation continued for another 2 h. Control glycopeptides were treated identically except that EDC/NHS solution was replaced with water. These carboxylate-neutralized glycopeptides (herein referred to as “asialo-CONHMe-glycopeptides”) were then dialyzed against water overnight to remove excess methylamine. Coupling to BSA was then carried out as above. Carboxylate neutralization using EDC-NHS/methylamine caused ˜70-80% of the negative charges to be masked as determined by QAE-Sephadex chromatography (data not shown).

### Example 3

### Immunization Procedures and Hybridoma Establishment

In the development of the antibodies of the present invention, screening was specifically conducted for IgG secreting hybridomas that reacted with total bovine lung glycopeptides coupled to BSA. Since the original immunogen contained only bovine glycans and no peptides, this strategy selectively detected only antibodies directed against the oligosaccharides, while avoiding detection of antibodies directed against streptavidin or BAP. Early on, it was noted that some of the antibodies reacted differentially, depending on whether the original coupling of the glycopeptides to BSA was done with glutaraldehyde (which reacts with amino groups) or carbodiimide (which reacts with carboxyl groups). It was reasoned that the decreased reactivity with carbodiimide-coupled glycopeptides might result from a carbodiimide-induced modification of the novel carboxylate on the glycans. In fact, it was found that binding of four of the most reactive antibodies (mAbEE4.1, mAbGB3.1, mAbB2.6, and mAbEH2.7) was also substantially abrogated by direct methylarnidation of the carboxylates on the target glycopeptides (FIG. 33). Of these, the focus was placed on mAbGB3.1, of IgG2b subclass, since it gave the best reactivity in blots and ELISAs. The protocols used in these experiments are discussed in more detail below.

Two four-week old female BALB/c mice were primed by subcutaneous injection of 10 μg native streptavidin in complete Freund's adjuvant. Seven days later, they were immunized with 10 μg streptavidin neoglycoconjugates and then boosted intraperitoneally with 10 μg neoconjugate in PBS (approximately 20 μl) at two week intervals, until significant titers of serum antibodies against BSA neoglycoproteins were generated. Splenocytes from the mice were isolated and fused with Ag8.653 mouse myeloma cells (kindly provided by Dr. Nissi Varki, at the University of California, San Diego) using standard protocols known in the art.

Hybridomas were cultured in Dulbecco's modified Eagle (high glucose) medium (GibcoBRL) supplemented with 100 U/ml penicillin, 100 μg/ml streptomycin, 2 mM L-glutamine, 20% fetal bovine serum and 100 μM hypoxanthine/16 μM thymidine. Screening was done by ELISAs against bovine lung glycopeptides coupled to BSA, thus selecting for antibodies directed against glycans and avoiding any against streptavidin and BAP.

In these ELISAs, 96-well plates were coated with 250 ng of BSA neoglycoproteins at incubated either for 6 h at room temperature, or 4 h at 37° C. Plates were washed and blocked with 3% BSA in PBS overnight, incubated with hybridoma culture supernatants or with purified IgG diluted in PBS containing 1% BSA and 0.05% Tween 20. Incubations were for 1-2 h at room temperature, or overnight at 4° C. The plates were then washed and incubated with alkaline phosphatase conjugated goat anti-mouse IgG, followed by development with p-nitrophenyl phosphate substrate. Absorbance was measured at 405 nm in an ELISA plate reader.

Seven highly reactive and 18 moderately reactive hybridomas were cloned by limiting dilution, and subcloned as necessary. The hybridomas were further screened for differential reactivity against BSA coupled asialo-COO⁻ glycopeptides and asialo-CONHMe-glycopeptides. Selected hybridoma clones/subclones were injected intraperitoneally into BALB/c mice for the production of ascitic fluid and IgGs were purified from culture supernatants or ascitic fluid on Protein G Sepharose, as known in the art. Four hybridoma suclones named EE4.1, GB3.1, B2.6, and EH2.7, were of particular importance since they generated, respectively, the monoclonal antibodies mAbEE4.1, mAbGB3.1, mAbB2.6, and mAbEH2.7 which showed the highest binding to immobilized carboxylated glycans, and whose binding to the carboxylated glycans was substantially abrogated by direct methylamidation of the carboxylates on the target glycopeptides (FIG. 33). One of the clones (mAbGB3.1, immunoglobulin subclass IgG2b) was chosen for further characterization, since it showed a marked difference in reactivity between asialo-COO⁻ glycopeptides and asialo-CONHMe-glycopeptides, and functioned well both in ELISAs and immunoblots.

mAbGB3.1 binding to immobilized BSA neoglycoproteins could be blocked by asialo-COO⁻ glycopeptides in solution, but not by asialo-CONHMe-glycopeptides (FIG. 2, Panel A). Similarly, FIG. 33 shows that the % binding of monoclonal antibodies EE4.1, GB3.1, B2.6, and EH2.7 that were raised against the carboxylated glycan, to immobilized BSA neoglycoproteins was blocked in the presence of asialo-COO⁻ glycopeptides (open boxes) in solution, but not by asialo-CONHMe-glycopeptides (closed boxes). The consequences of protonation of negative charges on binding by studying reactivity at different pH values were further analyzed.

As shown in FIG. 2, Panel B, a marked loss of reactivity was observed below pH 6.0, whereas the reactivities of two control monoclonal antibodies against their respective antigens were unaffected. The pH dependence of mAbGB3.1 reactivity therefore suggests either that the target carboxyl group has a pKa of ˜5.8, or that the antibody binding site has a critical residue with a pKa in this range. Regardless, the possibility that the antibody reactivity might be blocked by other carboxylic acids was pursued. Indeed, as shown in FIG. 2, Panel C, reactivity was inhibited by low mM concentrations of several aliphatic carboxylate-containing compounds, such as acetate, succinate, citrate, isocitrate and EDTA, roughly in proportion to the number of carboxyl groups on each (all compounds were tested at pH 7.5 where the respective carboxyl groups are ionized). On the other hand, similar concentrations of glucuronic, and galacturonic acids (FIG. 2, Panel C) or sialic, lactic or pyruvic acids (data not shown) did not inhibit binding. Overall, it appears that molecules carrying multiple aliphatic carboxylate groups were the most effective inhibitors.

In other studies conducted during the development of the present invention, it had been noted that a significant part of the sialidase-resistant negative charge in the bovine lung glycans could be explained by the presence of sulfate esters. These would not be neutralized by the methylamidation procedure. However, to verify that mAbGB3.1 did not cross-react with sulfated glycans, Western blots were carried out on thyroglobulin, which carries terminal Gal-3-sulfate and internal GlcNAc-6-sulfate, and on N-CAM, which is known to express the HNK-1 epitope carrying a terminal glucuronic acid-3-sulfate. The antibody mAbGB3.1 did not react with either of these glycoproteins. Chondrosarnine, chondroitin sulfate (tetramer and octamer), and hyaluronic acid (tetramer and octamer) at 1 to 5 mM also do not inhibit binding, indicating that glycosaminoglycan-like epitopes do not cross react. Reactivity in the ELISA assay was also unaffected by sulfated or phosphorylated sugars such as Glc-1-phosphate, Glc-6-phosphate, Man-6-phosphate, Gal-6-phosphate, GlcNAc-1-and GlcNAc-6-phosphates, Glc-6-sulfate, and Gal-6-sulfate (when tested at or below 5 mM; data not shown).

### Example 4

### Deglycosylation of Papain-Released Bovine Lung Glycopeptides with PNGase A/PNGase F

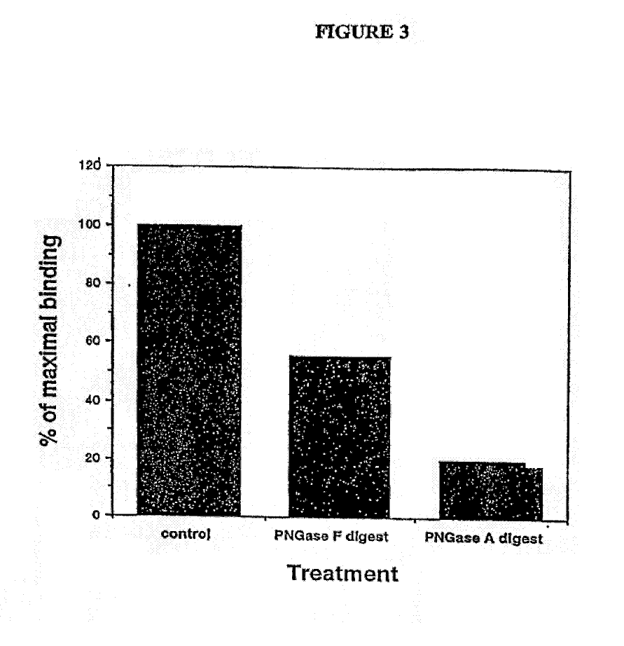

Bovine lung acetone powder was digested with papain in 0.1 M sodium acetate pH 5.5, in the presence of 5 mM L-cysteine and 1 mM EDTA at 60° C. for 48 h. The mixture was heat inactivated, centrifuged at 10,000×g to remove undigested residue, and dialyzed using 3000 molecular weight cut-off tubing. Glycopeptides (˜100 nmoles as determined by neutral sugar quantitation using phenol-sulfuric acid) were digested with PNGase A in 0.1 M sodium acetate or 0.1 M citrate-phosphate, pH 5.5, in the ratio of 2 μU/nmole of glycopeptide, or with PNGase F in 50 mM Tris, pH 8.0, for 16 h at 37° C., both in duplicate. Control tubes had no added enzyme. After digestion, the mixtures were heat inactivated. The glycopeptides with or without treatments were coated onto covalent binding plates and an ELISA against mAbGB3.1 was carried out as described above. Intact glycoproteins are poor substrates for PNGase A, but both enzymes can act well on glycopeptides released by thermolysin, pepsin, trypsin, or papain (Altmann et al., Glycoconjugate J., 12:84 [1995]). The deglycosylated peptides showed partial loss of antibody reactivity compared to undigested glycopeptides (incubated in the absence of enzymes). This loss of activity was greater with PNGase A digestion than with PNGase F (FIG. 3). It is contemplated that in some embodiments, this is because some of the carboxylate epitopes are present on PNGase F-resistant oligosaccharides carrying unusual core modifications.

### Example 5

### Immunostaining of Cells and Tissues

Various tissues were tested in these experiments to investigate the binding of novel glycoproteins to endothelial tissue. Tissue sections were fixed in 10% buffered formalin for 20 min, followed by blocking of non-specific binding sites with 10% normal goat serum in PBS containing 1% BSA. After washing, antibodies were overlaid onto serial tissue sections at predetermined dilutions (usually between 1-10 μg/ml) and the slides were incubated in a humid atmosphere for 30 min at room temperature or overnight at 4° C. The labeled streptavidin-biotin kit from Dako was used following the manufacturer's instructions with PBS or TBS washes between every step. Biotinylated anti-mouse IgG was applied for 10 min followed by either alkaline phosphatase or peroxidase-linked streptavidin for 10 min. After another wash, the appropriate substrate was added and the slides were incubated in the dark for 20 min. After a wash in buffer, slides were counterstained with hematoxylin, mounted and viewed using an Olympus BH2 microscope.

CPAE or HUVEC cells for cell-surface staining were grown on Lab-Tek Chamber glass Slides (Nunc), washed with PBS, and fixed in 10% buffered formalin for 20 min. The slides were blocked with 3% BSA in PBS for 20 min and then incubated with 10 μg/ml of either of the following Cy3 labeled antibodies, labelled according to the manufacturer's instructions: purified mAbGB3.1; anti human CD31 (positive control for endothelial cell staining; or CAB4 as a negative control (produced as described by Srikrishna et al., J. Biol. Chem., 272:25743 [1997]). After overnight incubation at 4° C., the slides were washed and observed using epifluorescence microscopy.

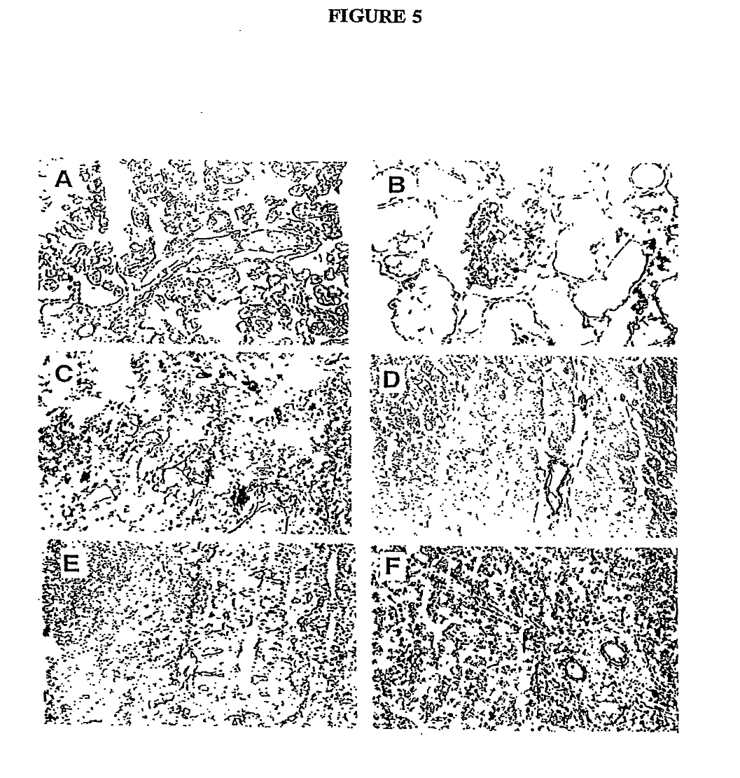

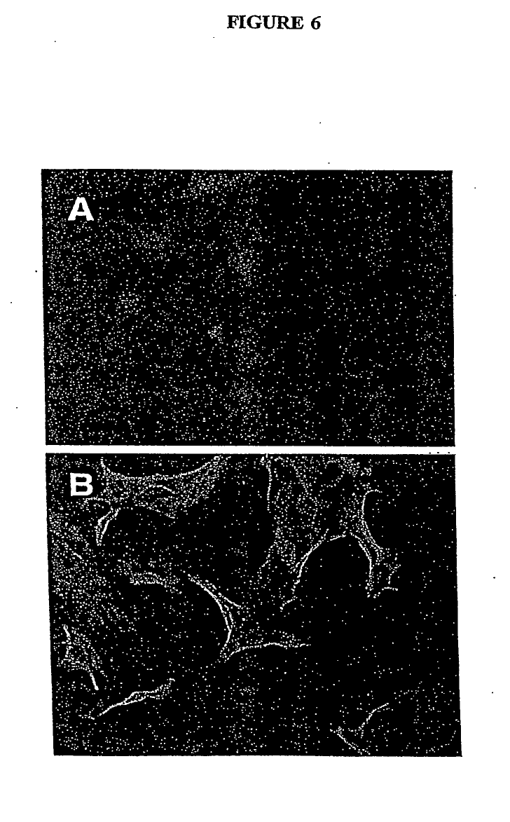

In frozen sections of bovine lung, the modification reported herein was localized mainly to the vascular endothelium, showing that the novel glycopeptides that were used in these experiments from whole bovine lung had primarily originated from the endothelium. Again, binding was almost completely blocked by asialo-COO⁻ glycopeptides (data not shown), confirming the specificity of the interactions. A more extensive survey of various human tissues showed that antibody reactivity was again predominantly localized to the vascular endothelium of most blood vessels (See e.g., FIG. 5). In the spleen and placenta almost every blood vessel was stained. At higher magnification of the skin vessels, it was evident that the endothelial cells were specifically stained (data not shown). The only positive non-endothelial cells were some lung macrophages. Staining of cultured human and bovine endothelial cells with Cy3 labeled mAbGB3.1 showed predominant localization of the epitope on the cell surface (See e.g., FIG. 6). The expression of the epitope was not upregulated after stimulation with activators such as TNFα or PAF (data not shown).

### Example 6

### Western Blot Analysis

Tissue or cell proteins were subjected to SDS/PAGE using 12% polyacrylamide gels under reducing conditions and transferred to nitrocellulose membranes. The membranes were blocked overnight with 10% skimmed milk or 3% BSA in PBS, washed with TBS containing 0.05% Tween20, and incubated for 1-2 h at room temperature with antibody from hybridoma culture supernatants (or purified IgG) diluted in PBS containing 1% BSA and 0.05% Tween 20. Membranes were then reacted with alkaline phosphatase-conjugated goat anti-mouse IgG, and reactive proteins visualized by incubating with the 5-bromo-4-chloro-3-indolyl phosphate/nitroblue tetrazolium substrate.

Western blots showed that several proteins from bovine lung and other bovine tissues reacted with mAbGB3.1. Reactivity was abrogated in the presence of lung asialo-COO⁻ glycopeptides, but not by asialo-CONHMe-glycopeptides (data not shown). There was also widespread reactivity in several human tissues (FIG. 4, Panel A). Binding to a majority of bands, except for the 38-40 kDa doublet bands in muscle tissue, was blocked by asialo-COO⁻ glycopeptides (FIG. 4, Panel B). Western blots of a variety of mouse and rat tissues were also positive, with almost all binding being blocked by asialo-COO⁻ glycopeptides (data not shown). This apparently widespread distribution of reactive epitopes is actually explained by the fact that they are concentrated on endothelial cells, which are of course present in all tissues (See, below).

### Example 7

### Isolation and Activation of Human Peripheral Blood Leukocytes

Since most of the antibody reactivity was localized to endothelial cells, it was reasoned that cognate lectins for these molecules might be found on peripheral blood leukocytes. Therefore freshly isolated human leukocyte populations (neutrophils, monocytes and lymphocytes) were probed with the BSA-coupled asialo-COO⁻ glycopeptides (to enhance multivalency, which is frequently required for lectin-carbohydrate interactions). Desialylated glycopeptides were used in order to eliminate any sialic acid-dependent binding. These experiments are described in more detail below.

Human neutrophils were isolated from fresh peripheral (EDTA-anticoagulated) blood of healthy volunteers by differential migration through mono-poly resolving medium (Ficoll-Hypaque d1.114) followed by hypotonic lysis of residual erythrocytes. The preparations were found to contain more than 98% granulocytes as confirmed using Turk's staining (0.01% crystal violet in 3% acetic acid). Cells were resuspended at 5×10⁶ neutrophils/ml in HBSS buffer containing 0.2% BSA and used within 30 minutes after isolation for adhesion assays. For flow cytometric assays, mononuclear cells from the above separation were further fractionated into monocytes and lymphocytes using Nycoprep media (the cell suspensions were approximately 80% pure). For activation, cells were resuspended at 3×10⁶/ml in 10 mM HEPES buffer pH 7.5 containing 150 mM NaCl, 5 mM KCl, 1.2 mM MgCl₂, 1.3 mM CaCl₂, and 5.5 mM glucose, and incubated in the presence or absence of 1 μM PAF (neutrophils), or 100 ng/ml PMA/1 μM ionomycin (neutrophils and mononuclear cells) for 15 min at 37° C. Cells were pelleted at 300×g for 10 min and analyzed using flow cytometry as described below.

Activated and unactivated peripheral blood leukocyte populations were incubated with 50 μg/ml BSA-conjugated asialo-COO⁻ glycopeptides or asialo-CONHMe-glycopeptides in HBSS buffer for 30 min on ice, followed by incubation with mouse monoclonal anti-BSA and R-phycoerythrin-conjugated anti-mouse IgG, each for similar lengths of time on ice and with two washes in between. Cells stained with second and third stage reagents only, or with BSA followed by second and third reagents, were used as negative controls.

In addition, neutrophils were analyzed by flow cytometric assay (i.e., FACS). Cells were resuspended at 3×10⁶ cells/ml in HBSS, incubated in the presence or absence of 1 μM PAF or 100 ng/ml PMA/1 μM ionomycin for 15 minutes at 37° C., and pelleted by centrifugation at 300×g for 10 minutes. Activated and unactivated neutrophils were incubated with mouse monoclonal anti-annexin I or anti-S100A8 (both IgG₁ mouse antibodies) or isotype IgG₁ mouse control antibody in HBSS containing 1% BSA (each antibody at a concentration of 1 μg/million cells), followed by FITC-conjugated anti-mouse IgG. The cells were then analyzed by FACS, using a FACScan (Becton-Dickinson) equipped with CellQuest software and gated by the side scatter and forward scatter filters.

S100A8 and S100A9 are cytosolic proteins, but membrane-associated forms have been identified in monocytes upon differentiation or activation (Roth et al., Blood 82:1875; and Mahnke et al., J. Leukoc. Biol., 57:63 [1995]). Thus, it was conceivable that neutrophil binding to immobilized glycans is due to translocation of A100A8/A9 complexes to the surface upon activation. To test this possibility, experiments were conducted to determine the surface expression of annexin I and S100A8/A9 before and after activation of neutrophils by FACS, as indicated above. S100A8 and annexin I are not expressed on resting neutrophils, but are detected on the cell surface within 15 minutes of activation with PMA/ionomycin (See, FIG. 18) or PAF (data not shown). This also explains the observation made during the development of the present invention that neutrophils acquire surface binding sites for the glycans upon short-term activation (Srikrishna et al., J. Immunol., 166:624 [2001]).

The specificity of this binding was proven by selective binding of BSA-coupled asialo-COO⁻ glycopeptides, but more than 80% reduction in the binding of BSA-coupled asialo-CONHMe-glycopeptides. It is contemplated that the residual binding of neutralized glycopeptides is due to the fact that carboxylate-neutralization by methylamidation does not achieve 100% completion even after two rounds of treatments. In addition to these surface binding sites, secretions elicited upon activation with other physiologically relevant inflammatory mediators such as TNFα or fMLP contained proteins that bound to the novel glycans in a carboxylate-dependent manner (data not shown). Subpopulations of monocyte and lymphocyte preparations also showed weaker staining after activation (data not shown).

For further characterization of binding, functional studies, and to verify whether the interactions are mediated by the proteins themselves or in association with other molecules, human S100A8/A9 and annexin I were purified.

### Example 8

### Neutrophil Adhesion Assays with Immobilized Carboxylated Glycans

Since a mouse monoclonal antibody had to be used to detect the BSA to which the glycopeptides were coupled, the effects of the mouse mAbGB3.1 could not be tested in the above flow cytometry assay. To address this issue and to obtain further evidence for specific binding, incubated freshly isolated neutrophils were incubated with BSA conjugated asialo-COO⁻ glycopeptides coated on plastic.

First, 96 well microtiter plates were coated with 250 ng/well of BSA coupled bovine lung asialo-COO⁻ glycopeptides for 4 h at 37° C. The plates were then washed and blocked with 3% BSA in PBS overnight. Control wells were coated with BSA alone. Cells were resuspended at 5×10⁶ neutrophils in 100 μl of HBSS buffer containing 0.2% BSA and 1 μM PAF were added to each well, and incubated for 30 min at 37° C. in the presence and absence of varying concentrations of mAbGB3.1 or a control antibody, or of asialo-COO⁻ or asialo-CONHMe-glycopeptides, in a total volume of 200 μl. PAF was excluded from the incubation mixture in some wells. Unbound cells were removed by flicking out the plates and washing five times with HBSS. Bound cells were quantitated by assaying myeloperoxidase. Briefly, adherent cells were solubilized in 100 μl of 1% Triton X100, and incubated with 25 μl of 0.5% hydrogen peroxide and 25 μl of 4.7 mM orthodianisidine for 30 min at room temperature. The reaction was stopped with 0.4% sodium azide, and absorbance was read at 450 nm. Assays were performed in duplicate.

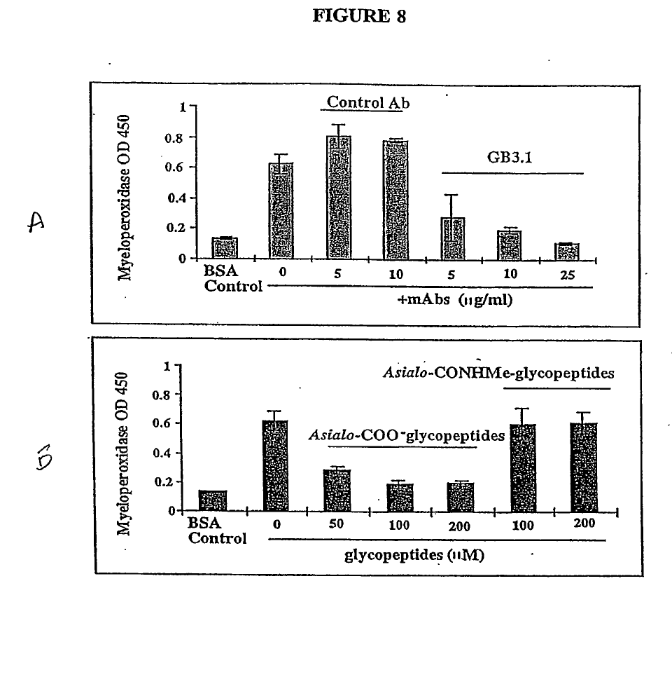

As shown in FIG. 8, activated human neutrophils bound to the carboxylated glycans, while minimal binding was observed to BSA itself. Adhesion was completely inhibited by mAbGB3.1 at various concentrations, whereas a control anti-carbohydrate antibody did not have any effect. Binding was also progressively and completely blocked by adding soluble bovine lung asialo-COO⁻ glycopeptides, but not by asialo-CONHMe-glycopeptides.

### Example 9

### Neutrophil Adhesion to Endothelial Cells

In this Example, experiments conducted to assess neutrophil adhesion to endothelial cells are described. HUVECs were grown to confluence in endothelial cell growth medium (Cell Applications), at 37° C. in 5% CO₂. Cells at passages 2-3 that were positive for cell staining and Western blotting with mAbGB3.1 were used in the adhesion assays. At confluence, HUVECs were detached and seeded on fibronectin (1 μg/cm²)-coated 96-well culture wells. Confluent EC monolayers were treated with medium containing μM PAF for 4 h at 37° C. and washed. In some experiments, unstimulated epithelial cells were also tested. Neutrophils isolated and suspended in adhesion buffer as described above were added and incubated under static conditions in the presence of PAF for 30 minutes at 30° C. in 5% CO₂, in the presence or absence of modifiers. Non-adherent cells were removed by washing and adherent cells were quantitated using the myeloperoxidase assay described above.

An isotype-specific control antibody did not have any effect on neutrophil adhesion to HUVECs. In contrast, increasing amounts of mAbGB3.1 enhanced adhesion by 2- to 3-fold (data not shown). This effect was observed regardless of whether the endothelial cells were prestimulated or the endothelial cells were incubated with the antibody before the neutrophils were allowed to adhere. Adhesion was also increased in the presence of asialo-COO⁻ glycopeptides, but not by asialo-CONHMe-glycopeptides. The reasons for the diametrically opposite effects of mAbGB3.1 and the soluble asialo-COO⁻ glycopeptides on adherence of neutrophils to plastic-immobilized glycans vs. endothelial cells are unclear. Presumably, the situation involving intact endothelial cells is more biologically complex and involves other signaling circuits and/or other adhesion pathways. For example, the interaction of activated neutrophils with the endothelial glycans could trigger cis or trans signaling in either or both of these cells, eventually resulting in inhibition of the overall interactions. Regardless, the results clearly indicate that these novel glycans on endothelial cells can modulate interactions with activated neutrophils. Moreover, an understanding of the mechanism(s) involved is not necessary in order to use the present invention.

### Example 10

### Zymosan-Induced Acute Peritoneal Inflammation

Although an understanding of the mechanism(s) is not necessary in order to use the present invention, experiments were conducted to examine the in vivo role of the novel glycans provided herein in the dynamic interaction between endothelial cells and leukocytes. In these experiments, the effects of mAbGB3.1 on the pathophysiology of acute inflammation were investigated. Since the novel glycan is also expressed in murine tissues, it was reasoned that the inflamed mouse peritoneum would be a convenient model to quantitatively examine such effects.

Female (BALB/c) mice (Harland, approximately 16-18 g body weight) were maintained on a standard chow pellet diet with tap water ad libitum, and used 2-3 days after arrival. The experimental protocols followed the criteria of the Institutional Animal Care and Use Committee at the Burnham Institute, La Jolla. Peritonitis was induced by intraperitoneal injection of 1 mg of zymosan in 0.5 ml PBS. Animals were injected intravenously with saline, mAbGB3.1, or isotype control mouse IgG at the time of zymosan injection. Mice were euthanized at different time points, and the peritoneal cavities were lavaged with RPMI medium containing 2% FBS, 3 mM EDTA and 25 U/ml heparin. Lavage fluids were centrifuged, aliquots of cells were stained with Turk's solution, and differential cell counts performed with a Neubauer hemocytometer. In parallel experiments, the peritoneal exudate cells were also immunostained with either FITC-labeled anti mouse Gr-1 (Ly6G) or PE-labeled anti-mouse Mac-1 for 20-30 min on ice, washed and analyzed by flow cytometry.

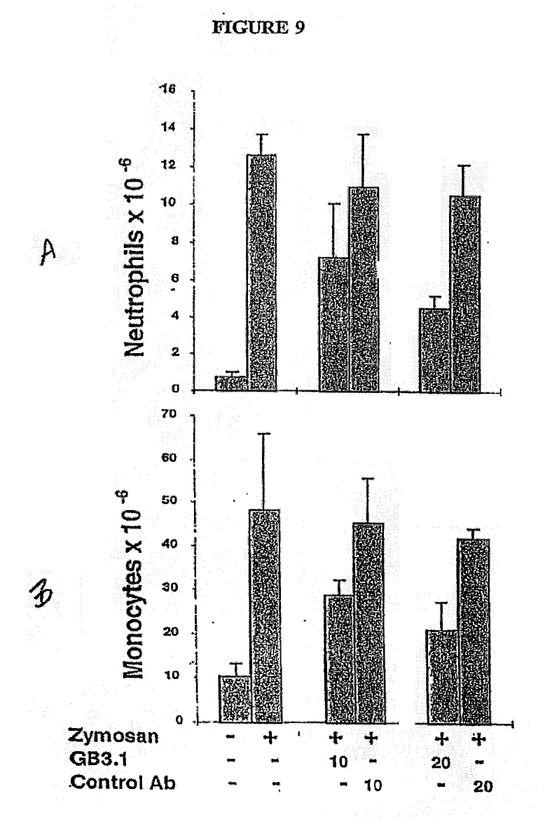

The mesenteries obtained from these animals were fixed in 10% buffered formalin, embedded in paraffin, stained with hematoxylin and eosin, and evaluated by brightfield microscopy using a Nikon microscope. Digital images were captured on a CCD camera at different magnifications and rendered with Adobe Photoshop. Zymosan-induced peritonitis in mice was characterized by a time-dependent accumulation of cells in the peritoneal cavity. Neutrophil content in peritoneal cavities rose from ˜0.75 million cells in saline-treated mice to ˜12 million cells within 4 hrs after zymosan challenge. Neutrophil influx was followed by monocyte infiltration which rose from ˜10 million cells in control mice to ˜50 million cells 16 hrs after zymosan treatment. Intravenous injection of mAbGB3.1 immediately prior to the induction of peritonitis resulted in a dose-dependent reduction in the extent of neutrophil and monocyte accumulation, while an isotype-matched mouse IgG or mAb AD7.5, or an unrelated anti-carbohydrate antibody (See e.g., Mehta et al., J. Biol. Chem., 271:10897 [1996], for a description of this antibody), had minimal effect, as shown in FIG. 9.

Titers of mAbGB3.1 checked at various time points after injection in control mice showed that antibody levels stabilized by 2 hrs, and remained steady up to 24 hrs in circulation (data not shown). The dose of 20 μg/g body weight which gave about 65% reduction in infiltration was chosen to further confirm the patterns of cells in the inflamed peritoneum by flow cytometry. Peritoneal cells from untreated and zymosan treated mice were stained with FITC-labeled anti mouse Gr-1 (anti-neutrophil) or PE-labeled anti-mouse Mac-1 (monocytes and neutrophils) and analyzed by flow cytometry. Four hours after zymosan injection, Gr-1^{high} neutrophils were the most abundant in the peritoneum, followed by recruitment of Gr-1^{low}/Mac-1^{high} monocytes at 16 hours (data not shown). Again, injection of mAbGB3.1 caused a substantial reduction in appearance of both types of cells, while a control antibody had no effect (data not shown).

Although an understanding of the mechanism is not necessary in order to use the present invention, it is contemplated that in some embodiments, decreased neutrophil and monocyte entry into the inflamed peritoneum upon injection of mAbGB3.1 results from a decreased ability to adhere to the endothelium, while in other embodiments, it is contemplated that a failure to emigrate after the cells have adhered is involved. To directly address this issue, mesenteric vessels from 4 hr and 16 hr time points were histologically examined for intravascular and extravascular accumulation of leukocytes. For both time points, zymosan injection caused extravascular migration of leukocytes, correlating with the increase in exudated cells in the peritoneal lavage fluids, which was unaffected by injection of a control antibody, as shown in FIG. 10, for the 16 hr. time point. In addition, increased leukocyte adherence to the mesenteric venules was also detected in saline or control antibody treated mice (See, FIG. 10). In contrast, mAbGB3.1 treated mice showed a clear reduction in leukocyte recruitment into the inflamed tissues. This effect of the antibody was associated with marked increase in adherence of cells to the endothelium in over half of randomly examined mesenteric venules, as shown in FIG. 10. Thus, the reduction in cell efflux into the peritoneal cavity is explained by a reduction in transmigration across the vessel walls. Although an understanding of the mechanism(s) is not necessary in order to use the present invention, it is contemplated that in some embodiments, the novel glycans selectively modulate adhesion and transmigration of activated neutrophils across the vascular endothelium in the setting of acute inflammation.

### Example 11

### Generation and Fractionation of Bovine Lung Glycopeptides

In these experiments, bovine lung glycopeptide preparations used in the remaining experiments were prepared. Five grams of bovine lung acetone powder (Sigma) was extensively digested with Proteinase K at 50° C., for 24 h in 0.1 M TrisHCl, pH 7.5. The mixture was then heat inactivated by boiling at 100° C. for 5 min, centrifuged (30 minutes at 10,000×g), and the supernatant was dialyzed against 3×4 L of water using a molecular weight cut-off of 3500 daltons. The glycopeptides were then loaded on a 75 ml column of DEAE Sephadex A25 equilibrated in 10 mM TrisHCl pH 8.0, washed with 10 column volumes of starting buffer, and eluted sequentially with 0.1 M, 0.3 M, and 1 M NaCl in starting buffer. These fractions are herein simply referred to as “0.1 M, 0.3 M or 1 M” glycopeptides. The eluates were individually dialyzed extensively against water using a molecular weight cut-off of 1000 daltons and tested for their ability to inhibit binding of mAbGB3.1 to immobilized bovine lung glycans.

Briefly, 96-well plates were coated with 50 ng of bovine albumin (BSA) neoglycoproteins containing total bovine lung glycopeptides coupled to BSA using glutaraldehyde for 4 h at 37° C. The neoglycoproteins were prepared for immunization screening as described above.

Plates were washed and blocked with 3% BSA in phosphate-buffered saline (PBS) overnight at 4° C. Wells were incubated with purified mAbGB3.1 IgG at 10 ng/ml in PBS containing 1% BSA and 0.05% Tween 20, in the absence or presence of varying concentrations of the above glycopeptides, in a total assay volume of 100 μl for 2 h at room temperature. The plates were then washed and incubated with alkaline phosphatase conjugated goat anti-mouse IgG, followed by development with p-nitrophenyl phosphate substrate. Absorbance was measured at 405 nm in an ELISA plate reader.

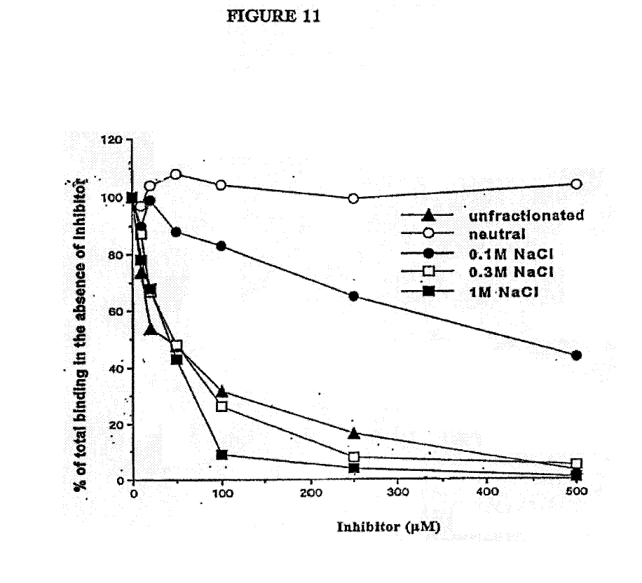

The results indicated that each of the anionic fractions inhibited binding of the antibody to bovine lung proteins, but the neutral glycopeptides did not (See, FIG. 11). The concentration of inhibitor glycopeptides was determined by neutral sugar estimation (assuming 5 hexoses per glycopeptide). Binding in the absence of inhibitor was considered to be 100%. The 1 M fractions gave variable results with different preparations (data not shown). Based on earlier work conducted during the development of the present invention, it is likely that the 1 M fractions probably included a complex array of glycoaminoglycan chains, and since the most consistent results were obtained with 0.1 M and 0.3 M fractions, these fractions were further processed. Also, for most inhibition assays described herein, desialylated 0.3 M glycopeptides were used at 100 to 200 μM final concentration.

Monosaccharide compositional analysis of the 0.1 M and 0.3 M glycopeptides obtained in a similar fractionation experiment were analyzed using an HPAEC-PAD system as known in the art (See, Norgard-Sumnicht et al., J. Biol. Chem., 270:27634 [1995]). Bovine lung glycopeptides obtained by charge separation were analyzed using a CarboPac PA1 column (Dionex). Sialic acid content was determined using a resorcinol assay as known in the art. The monosaccharide compositional analysis indicated that 0.1 M and 0.3 M fractions typically indicated that sugars commonly found in N- and O-glycans were present. The 0.1 M and 0.3 M glycopeptides contained approximately 18%, and approximately 11% carbohydrates by weight, respectively. The gradient used does not separate mannose and xylose. However, there was no evidence of any uronic acids typical of glycosaminoglycan chains. The following Table provides the results of these analyses.

TABLE 1
Monosaccharide Compositional Analysis
of Bovine Lung Glycopeptides

| | 0.1 M Glycopeptides | 0.3 M Glycopeptides |
|---|---|---|
| Monosaccharide | (nmoles/mg dry weight) | (nmoles/mg dry weight) |
| Sialic Acid | 269 | 140 |
| Fucose | 32 | 13 |
| GalNH₂ | 63 | 67 |
| GlcNH₂ | 128 | 95 |
| Gal | 161 | 112 |
| Man/Xyl | 97 | 31 |

In additional experiments to determine the proportion of the unknown carboxylate groups in the two fractions, oligosaccharides were released form the 0.1 M or 0.3 M glycopeptides by hydrazinolysis, desialylated by mild acid treatment and end-labeled with [³H]sodium borohydride. Labeled oligosaccharides were subjected to methanolysis to remove sulfate esters and convert the carboxylates to methyl esters. The neutralized oligosaccharides were then subjected to base-treatment to regenerate the carboxylate groups. Charge fractionation before and after the treatments showed that approximately 29% and 33% of the glycans in 0.1 M and 0.3 M fractions, respectively, contained the novel carboxylate species. This was further confirmed by QAE analysis of [³H]acetic anhydride labeled desialylated 0.1 M or 0.3 M glycopeptides before and after carboxylate neutralization by methylamidation using EDC/NHS and methylamine (results not shown).

### Example 12

### Generation of an Affinity Column for the Isolation of Cognate Proteins from Bovine Lung

In view of the results obtained in the previous Example (Example 11, and to eliminate any sialic acid-dependent binding, disialylated carboxylate-enriched glycopeptides were used to make an affinity column in this set of experiments. In these experiments, 0.1 M and 0.3 M NaCl eluates from the fractionation described in Example 11 were individually used to generate affinity columns The glycopeptides were first desialylated by mild acid treatment (10 mM HCl, 30 min at 100° C.) to remove sialic acids, split into two equal aliquots and lyophilized. These are referred to herein as “0.1 M or 0.3 M asialo-COO⁻ glycopeptides.” The carboxylate groups on one aliquot were modified by two cycles of methylamidation by EDC/NHS and methylamine as known in the art (See, Norgard-Sumnicht et al., supra) to produce negative controls. This treatment neutralized about 80% of the carboxylate groups (data not shown). These glycopeptides are referred to herein as “0.1 M or 0.3 M asialo-CONHMe-glycopeptides.” The glycopeptides were coupled to BSA using glutaraldehyde as described above. The reactivity of 0.1 M and 0.3 M BSA-asialo-COO⁻ glycopeptides against mAbGB3.1 was substantially reduced (approximately 75% and 95%, respectively) after carbohydrate neutralization (data not shown). These carboxylate-enriched or neutralized glycopeptide-BSA-neoglycoproteins were then individually coupled to Affigel-10, following the manufacturer's recommendations to produce the affinity columns. The efficacy of methylamidation of carboxylates was determined by loss of reactivity with mAbGB3.1.

Briefly, 96-well plates were coated with 250 ng of the individual BSA neoglycoproteins in PBS for 4 h at 37° C. Plates were washed and blocked with 3% BSA in PBS overnight at 4° C. Wells were incubated with purified mAbGB3.1 IgG at 10 ng/ml in PBS containing 1% BSA and 0.05% Tween 20, in a total assay volume of 100 μl for 2 h at room temperature. The plates were then developed as indicated above.

### Example 13

### Affinity Chromatography, and SDS-PAGE, and Western Blot Analysis of Bovine Lung Extracts

As it is contemplated that a high concentration of immobilized negatively charged sugars functions simply as an ion exchanger rather than an affinity matrix, such non-specific ionic interactions were minimized by using physiological ionic strength and pH (phosphate buffer in the presence of 150 mM NaCl) during the initial binding. Since citrate is an inhibitor of mAbGB3.1 binding to bovine lung proteins, the column was then eluted with 50 mM citrate in phosphate buffer (keeping the pH and ionic strengths unchanged). These experiments are described in more detail below.

In these experiments, 100 g wet weight of bovine lung was homogenized with a BioHomogenizer in PBS containing EDTA-free protease inhibitors, 10 mM DTT, 1 mM CaCl₂ and 1% NP40. The suspension was centrifuged at 650×g for 15 min, and then at 10,000×g for 30 min. The supernatant (approximately 2.5 g of protein) was first precleared by passing it over a 2 ml BSA-Affigel column, and equal volumes were then loaded on BSA-bovine lung glycopeptide affinity columns as prepared according to Example 12, above, or the corresponding carboxylate-neutralized columns run in PBS (10 mM phosphate, 150 mM NaCl) pH 7.5. Unbound proteins were washed out with ten column volumes of starting buffer, and bound proteins were eluted with 50 mM sodium citrate in 10 mM phosphate buffer (no change in pH or net ionic strength over loading/wash buffer). The eluates were then dialyzed, lyophilized, reconstituted in PBS, and analyzed on SDS-PAGE gels as described below.

Affinity purified bovine lung proteins were separated on 12% SDS-PAGE gels under reducing conditions and visualized by silver staining or by using Gelcode Blue. For immunodetection, proteins were electroblotted onto nitrocellulose membranes. The membranes were blocked overnight with 3% BSA in PBS, washed with PBS containing 0.05% Tween 20, and incubated with mouse anti-bovine annexin I, mouse anti-human S100A8, or mouse anti-human S100A9 diluted in PBS containing 1% BSA and 0.05% Tween 20 for 1-2 hr at room temperature. This was followed by incubation with alkaline phosphatase or peroxidase conjugated goat anti-mouse IgG. Bound proteins were visualized by incubating with BCIP/NBT, or using chemiluminescence detection reagents. For some experiments, (e.g., Example 24), the eluates were also tested for the presence of amphoterin using rabbit anti-amphoterin, as known in the art (See e.g., Parkkinen et al., [1993], supra).

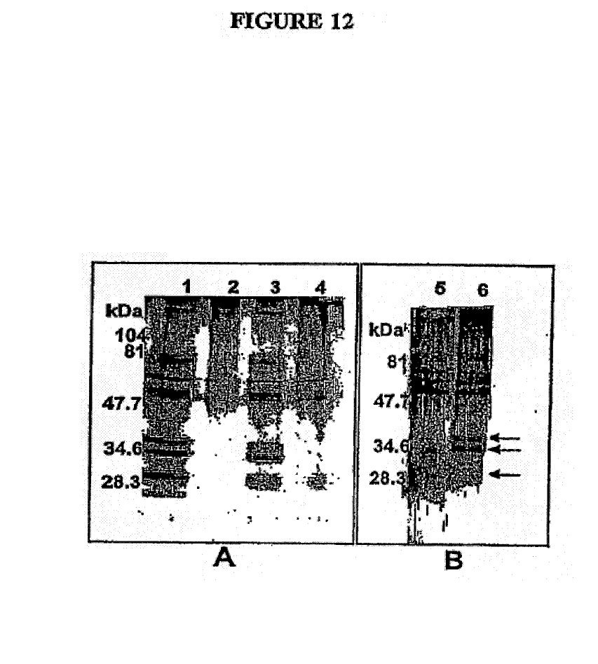

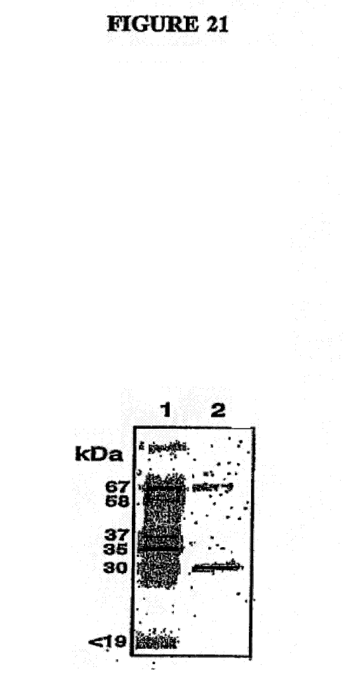

Under these conditions, less than 0.0001% of the loaded proteins bound to and eluted from the column. Of the five or more protein bands that were detected on silver gels, three proteins of masses 35, 37 and 30 kDa did not bind to the columns on which carboxylate groups were neutralized (See, FIG. 12, Panel A). As shown in FIG. 12, Panel B, the three proteins completely rebound to the unmodified column after dialysis to remove citrate, and could be eluted with as little as 5 mM citrate. Bands of >48 kDa were also present in the unbound fractions, and their binding was not carboxylate-dependent (FIG. 12, Panel B). As discussed in greater detail below, the 30 kDa protein was identified as amphoterin, based on its mass and anti-amphoterin reactivity (See, FIG. 21).

### Example 14

### N-Terminal Sequence Analysis

Bovine lung proteins eluted from the glycopeptide affinity column described above (Example 13) were separated on 12% SDS-PAGE run in electrophoretic buffer containing 0.1 M thioglycolate, and transferred to PVDF membranes. Transfer was carried out at 80 mV for 2 h, and the proteins were stained with 0.1% Coomassie brilliant blue in 40% methanol. Individual bands were excised after destaining in 40% methanol and 7.5% acetic acid, and N-terminal Edman sequencing was performed on a Model 494 Procise Sequencer (Applied Biosystems).

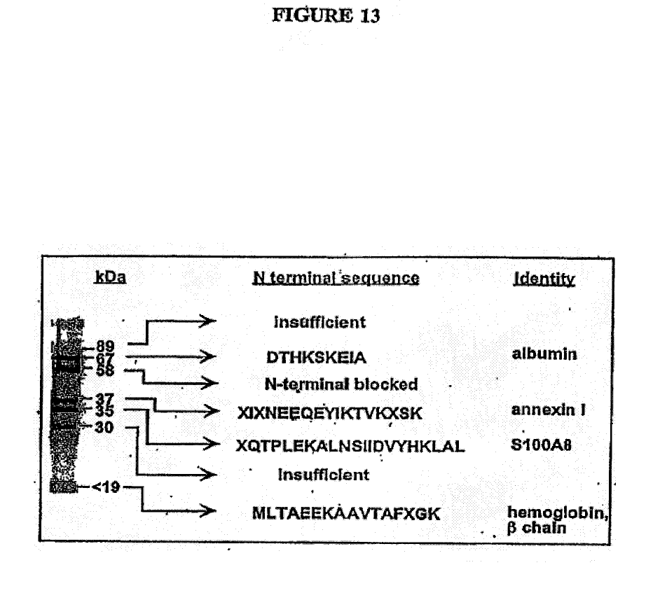

FIG. 13 shows the N-terminal sequences of the bound proteins. BLAST searches for sequence similarity of the proteins in the Genbank databases showed that the sequence of the 37 kDa band was >80% similar to a sequence beginning with the thirteenth residue of a leukocyte calcium-binding protein, annexin I. Native annexin I is known to be blocked at the N-terminus (See, Hall et al., Proc. Natl. Acad. Sci. USA 90:1927 [1993]). The identity of annexin I was further confirmed with immunoblots, using a commercially-available mouse monoclonal antibody directed against bovine annexin I. A doublet was recognized on the blots, comprising the native (37 kDa) and a “clipped” (approximately 35.5 kDa) form which is devoid of the first 12 amino acids, as deduced from protein sequencing data (not shown).

The N-terminal 21 amino acids of the 35 kDa band shared >80% sequence identity with an EF hand calcium-binding protein of the S100 family, namely migration inhibitory factor-related protein 8 (MRP8 or S100A8). Commercial monoclonal antibodies against distinct, but uncharacterized epitopes of human S100A8 did not recognize the bovine homologue (data not shown). S100A8 may not be a very conserved protein. For example, human S100A8 protein shares only about 59% sequence homology with that of the mouse (See, Lagasse and Clerc, Mol. Cell. Biol., 9:2402 [1988]; and Lagasse and Weissman, Blood 79:1907 [1992]). S100A8 has a molecular mass of 10.8 kDa. However, the protein isolated herein with the N-terminal sequence of S100A8 had a molecular mass of 35 kDa. It is known that the protein often exists and functions physiologically as a Ca²⁺-dependent complex with S100A9 (or MRP14), another protein of the S100 family (See, Teigelkamp et al., J. Biol. Chem., 266:13462 [1991]). The complexes have molecular masses of 24.5 kDa [S100A8•S100A9 heterodimer], 35 kDa [(S100A8)₂•S100A9 heterotrimer] and 48.5 kDa [(S100A8)₂•(S100A9)₂ heterotetramer] and usually break down in SDS gels under both non-reducing and reducing conditions. These complexes have been identified by ESI-MS (Strupat et al., J. Am. Soc. Mass. Spectrom., 11:780 [2000]) and the two-hybrid system (Propper et al., J. Biol. Chem., 274:183 [1999]). It is unknown how the 35 kDa [(S100A8)₂•S100A9] trimer was isolated as a non-dissociable complex. Sequence was not obtained for S100A9 on the 35 kDa band. This could be due to an N-terminal block, as is known with native human S100A9 (Kerkhoff et al., Biochim. Biophys. Acta 1448:200 [1998]). Table 2 provides a comparison of the N-terminal sequences of a few mammalian S100A8 and annexin I proteins. In this Table, the bovine sequences indicated with asterisks were obtained during the development of the present invention. These sequences are not shown in alignment.

TABLE 2
N-Terminal Sequences of Mammalian
S100A8 and Annexin I

| | | | |
|---|---|---|---|
| | GenBank | | |
| Protein | Acc. No. | Sequence | SEQ ID NO: |

S100A8

| | | | |
|---|---|---|---|
| Rabbit | D17405 | MPTDLENSLNSIIFVYHKC | SEQ ID NO: 5 |
| | | SL | |
| Mouse | X87966 | MPSELEKALSNITDVYHNY | SEQ ID NO: 6 |
| | | SN | |
| Human | M21005 | MLTELEKALNSIIDVYHKY | SEQ ID NO: 7 |
| | | SL | |
| Bovine* | | XQTPLEKALNSIIDVYHKL | SEQ ID NO: 8 |
| | | AL | |

Annexin I

| | | | |
|---|---|---|---|
| Rabbit | U24656 | MAMVSEFLKQAWFIDNEEQ | SEQ ID NO: 9 |
| | | DYINTVKTYK | |
| Mouse | M69250 | MAMVSEELKQARFLENQEQ | SEQ ID NO: 10 |
| | | EYVQAVKSYK | |
| Human | NM_000700 | MAMVSEFLKQAWFIENEEQ | SEQ ID NO: 11 |
| | | EYVQTVKSSK | |
| Bovine | X56649 | MAMVSEFLKQAWFIENEEQ | SEQ ID NO: 12 |
| | | EYIKTVKGSK | |
| Bovine* | | XIXNEEQEYIKTVKXSK | SEQ ID NO: 13 |

The 89 and 30 kDa protein bands were insufficient for sequencing. The 58 kDa protein was N-terminally blocked, and the 67 kDa protein was bovine albumin The sequence of the minor <19 kDa band was similar to the β chain of hemoglobin and a variable overlapping sequence which could not be matched with any known protein.

### Example 15

### Isolation of Human Neutrophils

For the remaining experiments involving neutrophils, human neutrophils were isolated from fresh peripheral (EDTA-anticoagulated) blood of healthy volunteers by differential migration through mono-poly resolving medium (Ficoll-Hypaque d1.114), followed by hypotonic lysis of residual erythrocytes. The preparations were >98% granulocytes as confirmed using Turk's staining (0.01% crystal violet in 3% acetic acid). Cells were lysed by sonication in PBS containing .protease inhibitors and debris was removed by centrifugation at 10,000×g for 30 minutes. Cells were resuspended at 5×10⁶ cells/ml in Hank's balanced salt solution (HESS) buffer containing 0.2% BSA and used within 30 min after isolation in adhesion assays. Specificity of binding was determined using three different approaches.

For activation, cells were resuspended at 3×10⁶ neutrophils/ml in 10 mM HEPES buffer pH 7.5 containing 150 mM NaCl, 5 mM KCl, 1.2 mM MgCl₂, 1.3 mM CaCl₂, and 5.5 mM glucose, and incubated in the presence or absence of 1 μM PAF or fMLP for 15 min at 37° C. The supematants were saved after pelleting cells by centrifugation. Where needed, soluble fractions were isolated by centrifugation at 100,000×g for 90 min after washing the cells and lysis in the above buffer.

### Example 16

### Binding of BSA-Glycopeptides Immobilized on ELISA Plates

Experiments were also conducted to determine whether human homologues of S100A8/A9 complexes and annexin showed similar binding, using three different approaches. First, the specific binding of the proteins from human neutrophil lysates to carboxylated but not to neutralized glycans was checked and confirmed by direct binding to immobilized glycans in ELISA (See, FIG. 14, Panels A and B). Next, as described in the following Examples, the binding of human neutrophil lysate proteins to the carboxylated and carboxylate-neutralized glycopeptide columns under the same conditions that were used for bovine proteins was analyzed.

In this Example, experiments conducted to determine the binding of human neutrophil lysates to BSA-glycopeptides immobilized on ELISA plates. First, 96-well plates were coated with 250 ng BSA coupled to 0.3 M asialo-COO⁻ or asialo-CONHMe glycopeptides in PBS for 4 hr at 37° C. The plates were washed and blocked with 3% BSA in PBS overnight at 4° C. The wells were then incubated with neutrophil lysate proteins in 10 mM HEPES buffer, pH 7.5, containing 150 mM NaCl, 5 mM KCl, 1.2 mM MgCl₂, and 1.3 mM CaCl₂, for 2 hr at room temperature. This was followed by incubation with anti-bovine annexin I antibody, anti-human S100A8, or anti-human S100A9 for 2 hr at room temperature. Wells were then washed and incubated with alkaline phosphatase conjugated goat anti-mouse IgG, followed by development with p-nitrophenyl phosphate substrate. The results are shown in FIG. 14.

### Example 17

### Binding to the Glycopeptide Affinity Columns

In this Example, experiments conducted to determine the binding of human neutrophil lysates to glycopeptide affinity columns are described. Lysate proteins (approximately 1 mg) were individually precleared on a BSA-Affigel column, and equal volumes were then loaded on BSA-bovine lung glycopeptide affinity columns or the corresponding carboxylate-neutralized columns run in PBS (10 mM phosphate, 150 mM NaCl), pH 7.5. Unbound proteins were washed out with 10 column volumes of starting buffer and bound proteins were eluted with sodium citrate in phosphate buffer, without change in ionic strength, as described above for bovine proteins. The unbound fractions and eluates were concentrated on YM10 membranes and equivalent proportions of each were analyzed after separation on 12% SDS gels by silver staining and Western blotting using mouse anti-bovine annexin I, mouse anti-human S100A8, or mouse anti-human S100A9, as described above for bovine proteins.

As indicated in the insets of FIG. 14, both S100A8/A9 and annexin I specifically bound to the carboxylated, but not the neutralized species. Bound proteins also consisted of few other minor bands that were detected on silver stained gels (data not shown). The identity of these were not determined. Approximately 95% of the total loaded proteins did not bind to the columns and since S100A8, S100A9, and annexin-I together account for approximately 35% of the total neutrophil cytosolic protein, the load probably exceeded the column capacity. Analysis was also conducted using reducing gels, to determine if any of the usually non-dissociable S100 complexes (i.e., as was observed with the bovine S100A8/A9 heterotrimer) were present in the bound fractions. In fact, a small fraction of the bound human S100 proteins consisted of a stable heterodimer, the identity of which was confirmed by a separate immunoblot with S100A9 (data not shown). However, a majority of the proteins broke down to monomeric forms. Also, the identity of a set of approximately 42-45 kDa anti-S100A8 reactive bands that bound to the column could not be determined, since they did not react with anti-S100A9, and their masses did not correspond to any known S100A8/A9 complexes.

### Example 18

### Depletion of Annexin I and S100 Proteins from Human Neutrophil Lysates by Incubation with Bovine Lung Glycopeptides

As the two affinity-isolated bovine proteins showed specific biding to bovine lung glycans immobilized on microliter plates, as detected by radiolabelled S100A8/A9 or anti-bovine annexin I antibody (data not shown), the following depletion assay experiments were conducted.

In these assays, extracts from activated human neutrophils were first incubated with immobilized asialo-COO⁻ glycopeptides, and the residual proteins in the supernatants were analyzed by immunoblots. Since the S100A8/A9 complexes are typically destroyed during SDS-PAGE (i.e., break down into monomers), the intensity of staining in immunoblots was beyond the linearity of reaction for any quantitative evaluation. Therefore, in order to determine if physiologically relevant multimeric complexes showed specific binding, the complexes were stabilized by chemical cross-linking before SDS-PAGE analysis.

In these experiments, 96-well plates were coated with 250 ng of BSA coupled 0.3 M asialo-COO⁻ lung glycopeptides for 4 h at 37° C. and blocked with 3% BSA in PBS overnight. Human neutrophil extracts were sequentially incubated in each of four wells for 30 min/well at 37° C. in leukocyte activation buffer (described above), in presence or absence of 200 μM 0.3 M asialo-COO⁻ glycopeptides or asialo-CONHMe-glycopeptides. After incubation, the supernatants from the incubation wells were collected and protein depletion was analyzed by immunoblotting using the respective antibodies. For detection of S100A8/A9 complexes, the proteins in the supernatants were stabilized by chemical cross-linking using bis-sulfosuccinimidyl suberate (BS³) as known in the art (See, Staros, Biochem., 21:3590 [1982]). Briefly, the cross linker was added from a fresh 2 mM stock solution to neutrophil lysates obtained as described above, to a final concentration of 200 μM, and the mixture was incubated at room temperature for 30 min. Reactions were quenched by the addition of 1 M Tris to a final concentration of 10 mM and samples were stored at −80° C., until they were assayed. Cross-linked samples were incubated with 2-mercaptoethanol prior to separation on SDS-PAGE. Since crude lysates were used in the cross-linking, it is conceivable that non-specific cross-linking could occur. Therefore, only the bands that were still strongly stained with increasing dilution of both anti-S100A8 and anti-S100A9 were identified.

As shown in FIG. 15, S100A8 homodimer, as well as S100A8/A9 heterodimer and heterotrimer were depleted from the incubation mixture after binding to the glycopeptides (lane 2). The binding of the heterodimer and homodimer, but not the heterotrimer, was blocked in the incubation was done in the presence of bovine lung 0.3 M asialo-COO⁻ glycopeptides, but not by asialo-CONHMe-glycopeptides (lanes 3 and 4, respectively). A separate immunoblotting with anti-human S100A9 confirmed the identity of the heteromeric complexes (lane 5). It is contemplated that the differences in the binding and stability of the human and bovine heteromeric complexes represent species differences. The S100A8 monomer, which arises from breakdown of the complexes on SDS gels, also showed similar binding specificity. The monomer probably does not represent the component involved in the actual binding, since complex formation is considered to be an essential prerequisite for the biological function of these S100 proteins. However, an understanding of the mechanism(s) involved is not necessary in order to use the present invention.

Residual proteins from the above incubations were also separately analyzed by immunoblots using anti-annexin I. As shown in FIG. 16, annexin I from the neutrophil extracts appeared as the native (37 kDa) and the clipped (˜35.5 kDa) doublet which is devoid of the first 12 amino acids, based on the sequencing data. While the intact protein was >90% depleted from the neutrophil preparations after binding to the carboxylate-enriched glycopeptides, the truncated protein was not (lane 2), suggesting that the N-terminal 1-12 amino acids may be important for optimal binding under these conditions. Reactivity of the intact human protein was blocked in the presence of 0.3 M asialo-COO⁻ glycopeptides (lane 3), suggesting specificity for the novel carboxylate.

### Example 19

### Purification of Human S100A8/A9 Complex From Human Peripheral Blood Neutrophils

As described in more detail below, S100A8/A9 was purified from fresh human neutrophil cytosolic fractions, since they contain abundant amounts of the proteins (30-45% of total cytosolic proteins; See, Edgeworth et al., J. Biol. Chem., 266:7706 [1991]). The high solubility of S100 proteins in ammonium sulfate was exploited, in order to obtain a preliminary enrichment of approximately 80%, before further purification of the proteins (to approximately 95% purity) on a hydrophobic column (See, Koike et al., J. Biochem., 123:1079 [1998]). The proteins remained as heteromeric complexes through purification, were even retained on YM10 and YM30 ultrafiltration membranes, and could not be separated into individual proteins without denaturation. The molecular masses of the purified proteins as determined by SDS gels was 11 kDa and 14 kDa for S100A8 and S100A9 respectively (data not shown). Purified S100A8/A9 complex showed the same specificity of binding as the proteins from the initial human neutrophil cytosol (FIG. 19, Panel A). Binding to BSA coupled asialo-COO⁻ bovine lung glycopeptides immobilized on microtiter plates was inhibited in presence of 0.3 M asialo-COO⁻ glycopeptides, but much less effectively by the asialo-CONHMe-glycopeptides. In FIG. 19, each point is the mean of two determinations.

All steps were carried out at 4° C., unless otherwise indicated. Ammonium sulfate precipitation was carried out as known in the art (See, van den Bos et al., Prot. Expr. Purif., 13:313 [1998]). Briefly, 100 ml peripheral blood was obtained from healthy volunteers and neutrophils obtained by Ficoll-Hypaque centrifugation as indicated above. The cells were lysed in PBS containing 1 mM DTT, 1% NP40 and protease inhibitors, and subjected to ultracentrifugation at 160,000×g for 30 min. The protein in the supernatant was adjusted to 2 mg/ml with the above buffer, and the solution stirred on ice. Solid ammonium sulfate was slowly added to a final concentration of 75% (w/v) and stirred for 1 h. The solution was then centrifuged at 10,000×g for 30 min and the supernatant dialysed against 4 L×3 of the above buffer for 24 h. The preparation was concentrated on YM10 membranes, and applied to a 10 ml column of Phenyl-650C resin equilibrated in 0.1% trifluoroacetic acid (See e.g., Koike et al., J. Biochem., 123:1079 [1998]). The unbound proteins were washed off with 3 column volumes of starting buffer and the bound proteins eluted with a gradient of 0-50% acetonitrile in the starting buffer. S100A8/A9 proteins eluted at 35-50% acetonitrile, and were >90% pure as determined by SDS gels. The pooled fractions were lyophilized and reconstituted in PBS containing protease inhibitors and stored at −20° C.

### Example 20

### Expression and Purification of Recombinant Human Annexin I

Annexin I purified from various mammalian tissues contains a significant proportion of protein proteolytically cleaved at the amino terminus (See, De et al., J. Biol. Chem., 261:13784 [1986]). In order to obtain sufficient quantities of full length annexin I for functional studies, the entire coding sequence of human annexin I was cloned into the expression vector pET23b as described below. As indicated, the expression construct was transformed into the E. coli strain BL21(DE3).

Nucleotide sequences encoding human annexin I were amplified by polymerase chain reaction (PCR) using a cDNA clone containing the complete coding sequence as template and the oligonucleotides Ann1_up: 5′-GTCGATAACATATGGCAATGGTATCAGAATTCC-3′ (SEQ ID NO:14) and Ann1_down: 5′-AACGTACTTCATATGTTAGTTTCCTCCACAAAGAGCC-3′ (SEQ ID NO: 15) as primers. The PCR was conducted using 94° C. for 2 minutes, followed by 30 cycles of denaturation at 94° C. for 20 seconds, annealing at 45° C. for 30 seconds, elongation at 70° C. for 1½ minutes, which was then followed by 10 minutes at 70° C. for extended elongation. This resulted in a 1067 basepair fragment containing the entire annexin I cDNA flanked by NdeI restriction sites. The fragment was digested with NdeI and cloned into the expression vector pET23b using standard procedures known in the art (See e.g., Sambrook et al., Molecular Cloning: A Laboratory Manual, Cold Spring Harbor Laboratory Press, Cold Spring Harbor, N.Y. [1989], for general description of the methods), resulting in the plasmid pWE151. Proper insertion and DNA sequences were confirmed by established DNA sequencing methods using primers oVW144: 5′-GAT GCT GAT GAA CTT-3′ (SEQ ID NO:16); oVW145: 5′-AAG TTC ATC AGC ATC-3′ (SEQ ID NO:17); oVW146: 5′-CCA CAA CTT CGC-3′ (SEQ ID NO:18); and oVW147: 5′-GCG AAG TTG TGG-3′ (SEQ ID NO:19). This revealed a conflict from the published sequence (GenBank accession number NM_{—}0007000) at position 288 (T₂₈₈G). The sequences of two other clones showed the same alteration. However, the T to a G does not alter the encoded amino acid and thus could be a polymorphism.

The expression construct was then used to transform Escherichia coli strain BL21 (DE3) (Promega), using methods known in the art. Three hours after induction with 1 mM isopropyl-1-thio-β-D galactopyranoside, the cells, grown in LB media supplemented with 100 μg/ml ampicillin, were harvested by centrifugation and resuspended in 25 mM Tris-HCl buffer, pH 8.0. Soluble fractions were obtained by freeze-thaw lysis in three to five cycles in the above buffer. The lysates were loaded on a DEAE Sephadex A25 column equilibrated with the above buffer, and the flow-through fractions were collected. The molecular mass of purified annexin I was determined by SDS gels and MALDI-TOF mass spectrometry (PerSeptive Biosystems).

Freeze-thaw lysates from the transformed E. coli contained full length annexin I with apparent molecular mass of 38 kDa as determined by SDS gels and MALDI mass spectrometry (data not shown). Since the N-terminal regions of annexins are extremely sensitive to proteolytic cleavage, rapid purifications with fewer steps are generally recommended for all annexins. In the preparation described herein, >95% enrichment from freeze-thaw lysates was possible using a DEAE column to which the protein did not bind. Purified recombinant annexin I bound to BSA-coupled 0.3 M asialo-COO⁻ bovine lung glycopeptides that were immobilized on microliter plates (See, FIG. 19, Panel B). Again, the specificity of this binding was demonstrated by a dose dependent inhibition in presence of asialo-COO⁻ glycopeptides, but not by asialo-CONHMe-glycopeptides.

### Example 21

### Enzyme-Linked Immunosorbent Assays (ELISAs) Using Purified Proteins

During the development of the present invention, it was determined that when the novel carboxylated glycans are immobilized on plastic, they support the binding of freshly activated human neutrophils. This binding was completely blocked by mAbGB3.1 and by soluble asialo-COO⁻ glycopeptides, but not by the asialo-CONHMe-glycopeptides. To determine whether this interaction was in fact mediated by annexin I or the S100, the effects of anti-annexin 1, anti-S100A8, and anti-S100A9 on the binding of neutrophils to BSA-conjugated 0.3 M asialo-COO⁻ were tested. As shown in FIG. 17, while anti-annexin I and anti-S100A8 had little or no inhibitory effects, anti-S100A9 significantly reduced binding, as did mAbGB3.1, indicating that neutrophil binding to the novel glycans may be principally mediated by S100A9.

In these adhesion tests, 96-well plates were coated with 0.3 M asialo-COO⁻ glycopeptide-BSA neoglycoproteins (250 ng/well) for 4 h at 37° C. (control wells had BSA alone) and blocked with 3% BSA in PBS overnight. Then, 0.5×10⁶ neutrophils in 100 1 oμf BHSS buffer containing 02% BSA and 1 μM PAF were added to each well, and the wells were then incubated for 30 minutes at 37° C., in the presence or absence of varying amounts of modifiers, in a total volume of 200 μl. Unbound cells were removed by washing five times with HBSS. Bound cells were quantitated by assaying for myeloperoxidase. Assays were conducted in duplicate.

In additional ELISA experiments, purified human annexin I or S100A8/A9 complexes (as appropriate), in 10 mM HEPES buffer pH 7.5 containing 150 mM NaCl, 5 mM KCl, 1.2 mM MgCl₂ and 1.3 mM CaCl₂, in the absence or presence of varying concentrations of 0.3 M asialo-COO⁻ glycopeptides or 0.3 M asialo-CONHMe-glycopeptides for 2 h at room temperature. This was followed by incubation with anti-bovine annexin I antibody or anti-human S100A8 or anti-S100A9 for 2 h at room temperature. Wells were then washed and incubated with alkaline phosphatase conjugated goat anti-mouse IgG, followed by development with p-nitrophenyl phosphate substrate.

For further characterization of binding and functional studies, and in order to verify whether the interactions are mediated by the proteins themselves or in association with other molecules, human S100A8/S100A9 and annexin I were purified and used in other experiments, as described herein.

In additional ELISA experiments, 96-well plates were coated with 0.3 M asialo-COO⁻ glycopeptide-BSA neoglycoproteins (250 ng/well) for 4 h at 37° C. (control wells had BSA alone) and blocked with 3% BSA in PBS overnight. The wells were then incubated with purified human annexin I or S100A8/A9 complexes (as appropriate), in 10 mM HUES buffer pH 7.5 containing 150 mM NaCl, 5 mM KCl, 1.2 mM MgCl₂ and 1.3 mM CaCl₂, in the absence or presence of varying concentrations of 0.3 M asialo-COO⁻ glycopeptides or 0.3 M asialo-CONHMe-glycopeptides for 2 h at room temperature. This was followed by incubation with anti-bovine annexin I antibody or anti-human S100A8 or anti-S100A9 for 2 h at room temperature. Wells were then washed and incubated with alkaline phosphatase conjugated goat anti-mouse IgG, followed by development with p-nitrophenyl phosphate substrate.

### Example 22

### Precipitation of Mannose-Labeled Glycoproteins

In previous Examples, it was shown that a major fraction of the mAbGB3.1-reactive carboxylated epitopes are carried on N-linked glycans, and that mAbGB3.1 recognized several proteins on endothelial cells. These experiments, mAbGB3.1 was tested to determine whether it consistently immunoprecipitates mannose-labeled glycoproteins. Further, to determine if the same glycoproteins were also recognized by the S100A8/A9 complex or annexin I, cross-binding assays were carried out as described in more detail below.

Calf pulmonary artery endothelial cells (CPAE) cells were labeled with [2-³H] mannose at 20 μCi/ml for 24 h in alpha MEM containing 10% fetal bovine serum. Cells were harvested by trypsinization, washed thrice in PBS, and lysed in PBS containing protease inhibitors and 0.5% NP40. Protein-rich lysates were prepared by ultrafiltration on YM10 membranes. Purified S100A8/A9 or annexin I proteins were immobilized on Affigel-10, and mixed with the labeled glycoproteins from CPAE cells in 10 mM HEPES buffer pH 7.5 containing 150 mM NaCl, 5 mM KCl, 1.2 mM MgCl₂ and 1.3 mM CaCl₂ after preclearing the lysates with BSA-Affigel 10. For immunoprecipitations using mAbGB3.1, lysates were precleared with normal mouse IgG-protein G beads and then incubated with Protein G coupled to mAbGB3.1 IgG in PBS. After overnight incubation at 4° C., the individual pellets were washed free of all unbound label until no more counts appeared in the washes, and the radioactivity associated with the pellets was measured.

For cross-binding, label associated with each pellet was first eluted using high salt (5 M MgCl₂, which eluted >70% bound label). The eluates were repeatedly desalted on YM10 membranes and resuspended in the respective buffers. Label eluted from mAbGB3.1 gels was mixed with immobilized S100A8/A9 or annexin I and vice versa. Again, after overnight incubation at 4° C., the individual pellets were washed free of all unbound label and counted. Table 3 below, provides data for immunoprecipitation experiments with mAbGB3.1 As shown in Table 3, S100A8/A9 complex precipitated 9% of mannose-labeled glycoproteins from CPAE cells, as compared to 14% of label immunoprecipitated by the antibody. When the precipitated proteins were eluted either from the S100A8/A9 complex or mAbGB3.1, and cross-bound, S100A8/A9 complex precipitated about one-third of the label eluted from mAbGB3.1, while mAbGB3.1 immunoprecipitated almost two-thirds of the label eluted from the S100A8/A9 complex. These experiments showed that the S100A8/A9 complex bound to a subset of endothelial glycoproteins recognized by mAbGB3.1. In comparison, immobilized annexin I precipitated only about 2-3% of mannose labeled glycoproteins from endothelial cells (data not shown). It is contemplated that this may either indicate differences in binding affinities, or suggest that more of the annexin I-reactive epitopes could be present on O-glycans.

TABLE 3
S100A8/A9 Complex Recognizes a Subset of [2-³H]Mannose-
Labeled CPAE Glycoproteins Immunoprecipitated by mAbGB3.1

| | | [2-³H]Mannose-labeled CPAE | | |
|---|---|---|---|---|
| Sample Processing | | Glycoproteins (cpm) | | % Bound |
| Primary | Rebinding | Total | | Radioactivity |
| Binding To: | to: | Added Gel | Bound to: | (mean ± SD) |
| mAbGB3.1 | — | 22,949 | 2,674 | 13.5 ± 1.8 |
| | | 22,565 | 3,132 | |
| | | 11,160 | 1,768 | |
| | | 11,206 | 1,420 | |
| S100A8/A9 | — | 20,386 | 1,698 | 8.7 ± 0.5 |
| | | 10,286 | 928 | |
| mAbGB3.1 | S100A8/A9 | 4,951 | 1,569 | 31.5 ± 0.2 |
| | | 1,961 | 615 | |
| S100A8/A9 | mAbGB3.1 | 1,074 | 693 | 62.5 ± 2.8 |
| | | 504 | 305 | |

### Example 23

### Surface Binding of Iodinated S100A8/A9 to Endothelial Cells

Human umbilical vein endothelial cells (HUVECs) were grown in Endothelial Cell growth Medium (Cell Applications) at 37° C. in 5% CO₂. Cell staining and Western blotting were performed to confirm expression of mAbGB3.1 antigens. HUVECS at passages 3-4 were used in the binding assays. Purified proteins were radioiodinated using Na¹²⁵I and Iodo-Beads Iodinating Reagent as per the manufacturer's protocols. Unbound label was removed on a Sephadex G25 column, followed by concentration of the void volume fractions using Centricon10 concentrators.

Cells grown to confluence in 12 well plates and activated with 1 μM PAF for 4 hr. Activated and unactivated cells were washed with cold HBSS and then incubated with 4 nM to 80 nM ¹²⁵I-S100A8/A9 in 1 ml of HBSS, or 20 nM labeled complex in the absence and presence of modulators at 4° C. for 2 h. The reaction was stopped by rapid removal of unbound label, and the cells were washed thrice with cold HBSS. They were solubilized in 1 M NaOH followed by addition of an equal volume of 1 M HCl, and the radioactivity associated with the lysates was measured using a gamma counter. FIG. 20 shows the results obtained in these experiments. Panel 1 shows the results for HUVECs incubated with 4 nM to 80 nM ¹²⁵I-S100A8/A9. Panels 2 and 3 show the results for HUVECs incubated with 20 nM labeled complex in the absence or presence of modulators, in 1 ml of HBSS at 4° C. for 2 hours.

As indicated in FIG. 20, dose-dependent and saturable binding of ¹²⁵I-S100A8/A9 on PAF-activated HUVECs was observed, as shown in Figure (FIG. 20, Panel A). Binding to unactivated cells was 2-fold less (data not shown). This binding was specific since it was competed by increasing amounts of unlabeled ligand, with half maximal competition of specific binding at approximately 500 nM (FIG. 20, Panel B), and also by mAbGB3.1, the blocking being as effective as competition using unlabeled ligand, while a control antibody had minimal effect (FIG. 20, Panel C). Binding was also inhibited by anti-S100A8 and anti-S100A9 (data not shown).

Efforts to identify annexin I binding sites on endothelial cells using radiolabeled annexin I were hampered by degradation of annexin I upon iodination, a problem also reported previously (See, Goulding et al., Biochem. J., 316:593 [1996]). Biotinylation did not destroy the protein, but abolished binding of both annexin I and S100A8/A9 to the glycans, (data not shown) suggesting involvement of critical lysines in the interactions.

### Example 24

### Affinity Chromatography

In these experiments, affinity chromatography was performed as described above in Example 13. Briefly, affinity columns containing immobilized COO⁻ glycans, and purification and analysis of bound lectins were produced and used as described above. BSA neoglycoproteins generated as described above were coupled to Affigel-10. Fresh bovine lung homogenates prepared as described earlier, were first precleared by passing over a 2 ml BSA-Affigel column, and equal volumes were then loaded on BSA-COO⁻ glycopeptide affinity column or the corresponding CONHMe-glycopeptide column run in PBS (10 mM phosphate, 150 mM NaCl), pH 7.5. Unbound proteins were washed out with ten column volumes of starting buffer, and bound proteins were eluted with 50 mM sodium citrate in 10 mM phosphate buffer (no change in pH or net ionic strength over loading/wash buffer). The eluates were then dialyzed, lyophilized, reconstituted in PBS, and analyzed on SDS-PAGE gels/Western blots. Amphoterin was detected in the affinity purified bovine lung proteins using rabbit anti-amphoterin (Parkldnen et al., [1993] supra).

As discussed above, a non-selectin ligand-based pathway functions during acute inflammation. The critical feature of this mechanism is a previously unknown carboxylate modification on endothelial cell glycoproteins. Thus, as indicated herein, during the development of the present invention lectins that might mediate these endothelial-leukocyte interactions were sought. As indicated in Example 13, a very small fraction of the proteins detergent-solubilized from whole bovine lung bound to affinity columns with immobilized carboxylated glycans. This included three proteins of masses 35, 37 and 30 kDa which did not bind to columns on which the carboxylate groups were converted to methylamides. The 35 and 37 kDa bands were S100A8/A9 and annexin I, respectively, as discussed above. The 30 kDa protein was identified as amphoterin, based on its mass and anti-amphoterin reactivity (See, FIG. 21).

Native amphoterin from human erythroleukemia cell lysates, and purified recombinant amphoterin (Parkkinen et al., [1993], supra) also bind to immobilized glycans in a carboxylate dependent manner (data not shown). Amphoterin binds to chondroitin-sulfate, heparin, and heparin sulfate proteoglycans (Miley et al., J. Biol. Chem., 273:6998-7005 [1998]; Rauvala and Pihlaskari, [1987], supra; and Salmivirta et al., Exp. Cell Res., 200:444-451 [1992]). However, desialylated carboxylate-enriched glycopeptides that were used in the glycan affinity column and in binding assays are moderately anionic and composed of sugars commonly found in N- and O-glycans. Their compositional analysis also shows no evidence for the presence of any uronic acids typical of glycosaminoglycan (GAG) chains. This rules out amphoterin binding to any potential GAG chains in the preparations.

### Example 25

### Immunoassays for Amphoterin Binding

In this Example, experiments conducted to assess amphoterin binding to COO⁻ glycopeptide coated plates was assayed as described earlier for annexin I and S100 proteins. In addition, bound protein was detected using anti-amphoterin. mAbGB3.1 binding to sRAGE was assessed by first coating sRAGE on microtiter wells for 4 h at 37° C. After blocking with 3% BSA in PBS overnight, wells were incubated with mAbGB3.1 in the presence and absence of COO⁻or CONHMe-glycopeptide and developed as described above.

Soluble RAGE (sRAGE), consisting of only the extracellular domain, was found to bind to mAbGB3.1. Soluble COO⁻ glycopeptides, but not CONHMe-species blocked binding at low concentrations (See, FIG. 22, Panel A). Purified oligosaccharides released from the glycopeptides by hydrazinolysis inhibited equally well (data not shown), showing that this effect did not depend on the peptides from the proteins.

### Example 26

### Overexpression and Purification of Recombinant RAGE Fragment

The internal BamHI sites of human RAGE cDNA were used to subclone the fragment coding for amino acids 90 to 347 into expression vector pQE-32. Overexpression of the recombinant RAGE with an N-terminal His-tag was performed in E. coli M15p (REP4), as recommended by the manufacturer. RAGE was purified from cell lysates on Ni-NTA-resin and purity assessed by SDS-PAGE and Western blotting using mouse anti-penta-His mAb using methods described above.

While overexpression of RAGE enhances tumor growth and metastasis, anti-RAGE, expression of dominant negative RAGE lacking a cytoplasmic tail, or addition of soluble RAGE prevent tumor growth and metastasis in mice. These effects are brought about by three co-existing MAPK molecules, namely p38, JNK, and p42/p44 (Taguchi et al., [2000], supra).

### Example 27

### Generation and Characterization of anti-RAGE Monoclonal Antibody

Recombinant RAGE fragment was used to immunize BALB/c mice and hybridomas were generated according to standard procedures. The hybridomas were screened against recombinant RAGE fragment, and positive colonies were cloned. One IgG2a (mAbA11) recognizing recombinant RAGE fragment, bovine RAGE, and High Five insect cells stably transfected with human full length RAGE cDNA (cloned into vector pIZ/V5-His) was used in the following experiments as culture supernatant. This antibody was used in various analyses of RAGE expression.

### Example 28

### Immunoprecipitation and Deglycosylation of RAGE

Bovine lung homogenates were prepared as described above. A 50% ammonium sulfate precipitation was carried out for enrichment of RAGE. For immunoprecipitations using mAbGB3.1, enriched homogenates were precleared with normal mouse IgG immobilized on Affigel 10 beads, followed by incubation with mAbGB3.1 immobilized beads. After overnight incubation at 4° C., the individual pellets were washed several times to remove unbound material. RAGE was released from the immunoprecipitate by 0.1 M triethanolamine, pH 11.5 or 0.1 M glycine, pH 2.5, and neutralized using 1 M Tris-HCl, pH 7.5. The protein was digested using PNGaseF (New England Biolabs) according to manufacturer's protocol, but where necessary, effective deglycosylation of RAGE was achieved without protein denaturation.

Native RAGE from bovine lung was immunoprecipitated with mAbGB3.1. Bovine lung was used as the source since it has the highest level of RAGE expression, and was also the original source of the carboxylated glycans (Brett et al., Am. J. Pathol., 143:1699-1712 [1993];. and Norgard-Sumnicht et al., J. Biol. Chem., 270:27634-27645 [1995]).

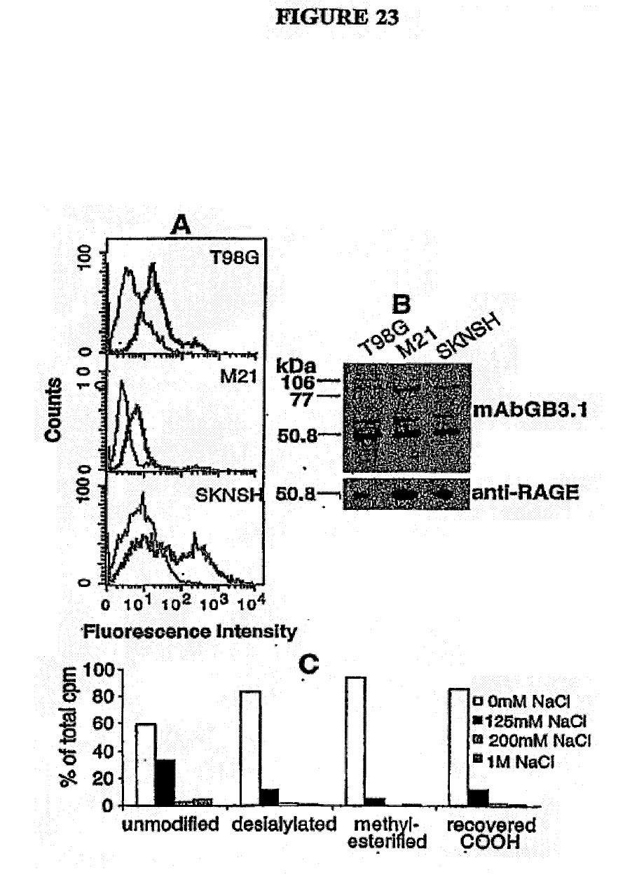

Low or high pH buffers release bound RAGE from the antibody, as do 200 mM COO⁻ glycans alone at neutral pH (data not shown), showing that the antibody-RAGE interaction is carbohydrate-based. Anti-peptide RAGE and mAbGB3.1 both recognize immunoprecipitated RAGE. PNGaseF digestion eliminates mAbGB3.1 binding and decreases the protein mass by about 4500 daltons, consistent with the loss of two N-linked oligosaccharide chains (See, FIG. 22, Panel B). RAGE is expressed in early neuronal development, downregulated in adulthood, and re-expressed in many types of tumors (Schmidt et al., Trends Endocrinol. Metab., 11:368-375 [2000a]). Carboxylated glycans were found to be expressed on the surface of many tumor cells, especially glioblastomas and neuroblastomas (See, FIG. 23, Panel A). In fact, RAGE was by far the most prominent mAbGB3.1 reactive band in these and several other tumor cells that we examined (See e.g., FIG. 23, Panel B). mAbGB3.1 immunoprecipitates 65% of RAGE from bovine lung, whereas more than 90% of RAGE from neuroblastoma cells is immunoprecipitable (data not shown). The extent of precipitation may depend on the precise structure of the sugar chains, or number or density of epitopes. It may also be cell-type specific.

### Example 29

### Analysis of Tumor Cells

Tumor cells were grown in DMEM (high glucose, Gibco BRL) containing 10% fetal bovine serum, 2 mM L-glutamine, 100 units/ml penicillin and 100 μg/ml streptomycin, and 100 μM non-essential amino acids. Cells were detached by incubation with PBS and 10 mM EDTA, washed thrice with ice-cold HBSS containing 1% BSA (staining buffer) and incubated with mAbGB3.1. Bound antibody was detected with FITC-conjugated goat anti-mouse Ig secondary antibody. Flow cytometry was performed on a Becton Dickinson FACscan equipped with CellQuest software. Proteins from whole cell lysates or membrane preparations were also analyzed by Western blotting using anti-RAGE or mAbGB3.1.

### Example 30

### Metabolic Labeling of RAGE and Analysis of Labeled Oligosaccharides

SKNSH human neuroblastoma cells were grown as described in Example 29, above. Metabolic labeling of cells using [2-³H] mannose and immunoprecipitation of labeled RAGE were done as described above. N-linked carbohydrate chains were released from the protein using PNGaseF. Analysis of oligosaccharides by Concanavalin A lectin affinity chromatography, desialylation, methylesterification to neutralize carboxylates and to remove sulfate esters, and QAE-Sephadex fractionations were performed as known in the art (See, Etchison et al., J. Biol. Chem., 270:756-764 [1995]; Norgard-Sumnicht et al., J. Biol. Chem., 270:27634-27645 [1995]). Saponification (0.1 N NaOH, 2 h, 50° C.) was used to regenerate the free carboxylate from the methylesters followed by neutralization with 1 M HCl.

More than 80% of the ³H label on mAbGB3.1 immunoprecipitated proteins from these cells was associated with RAGE (data not shown). Concanavalin A affinity chromatography of the PNGaseF-released sugar chains showed that about 25% of the label occurred in tri- and tetraantennary chains, 10% in biantennary and 15% in hybrid-type chains, with 50% in high mannose type species (data not shown). Based on the 2-3-fold higher mannose content in high mannose-type oligosaccharides, each RAGE molecule is therefore likely to have at least one complex-type N-linked glycan chain. QAE-Sephadex chromatography was used to select chemical modifications to identify the anionic substituents. In replicate experiments, approximately 42% of the radiolabel was anionic without any treatment, and 15-17% remained charged after desialylation with mild acid that removes sialic acid (FIG. 23, Panel C). The remaining charges were nearly all neutralized by methanolysis, which cleaves sulfate esters and converts carboxylates to neutral methylesters. Base hydrolysis of the desialylated, methanolyzed material, almost completely regenerates anionic species seen on desialylated oligosaccharides, consistent with reconversion of the methyl ester into the carboxylate. This provides further chemical evidence that RAGE oligosaccharides contain these unusual carboxylate groups. Amphoterin is identical to a previously described sulfoglycolipid binding protein-1 (SBP-1), which recognizes 3-sulfoglucuronyl β1-3 galactoside or HNK-1 (Chou et al., J. Neurochem., 77:120-131 [2001]; and Nair and Jungalwala, J. Neurochem., 68:1286-1297 [1997]). It was therefore considered possible that amphoterin-RAGE binding simply involves HNK-1 glycans on RAGE.

### Example 31

### Anti-HNK-1 Reactivity of RAGE

HNK-1 epitope content of RAGE was estimated by comparing anti-HNK-1 binding to an equivalent amount of HNK-1-reactive proteins secreted by CHO Lec2 cells co-transfected with glucuronyl and HNK-1 sulfotransferase (See, Ong et al., supra). As indicated in Example 30, it was considered possible that amphoterin-RAGE binding simply involves HNK-1 glycans on RAGE.

However, bovine lung RAGE did not react with HNK-1 antibody under conditions that can easily detect 0.1 pmole HNK-1/nmole of protein (data not shown). Therefore, it appears that RAGE does not contain this epitope. In addition, mAbGB3.1 does not recognize HNK-1 carbohydrates and the carboxylate-enriched bovine lung fractions used to prepare the affinity columns do not contain HNK-1 epitope or GAG chains either (data not shown). Furthermore, oligosaccharide analysis shows that RAGE contains complex type anionic N-glycans with non-sialic acid carboxylate groups, but not the HNK-1 (3-sulfoglucuronyl b1-3 galactoside) epitope.

### Example 32

### Binding Assays Using ¹²⁵I Amphoterin

Amphoterin was radioiodinated using Na ¹²⁵I and Iodo-Beads Iodinating Reagent (Pierce) to a specific activity of 5×10³ cpm/ng protein. Saturation binding experiments were done by a slight modification of the method of Hori et al (Hori at al., J. Biol. Chem., 270:25752-61 [1995]). Briefly, purified RAGE was immobilized on microliter wells and the wells blocked with 3% BSA. Wells were incubated at 4° C. overnight with increasing concentrations of ¹²⁵I amphoterin in HBSS containing 0.1% BSA in the presence or absence of desialylated carboxylate-enriched (COO⁻) or CONHMe bovine lung glycopeptides, or 20 μg/ml mAbGB3.1 or an unrelated anti-carbohydrate monoclonal antibody, or 100 fold excess of unlabeled amphoterin. Binding was also tested on RAGE deglycosylated by PNGase F under non-denaturing conditions. Wells were washed, bound amphoterin was eluted with 2 M NaCl, and radioactivity was measured using a gamma counter. Non-linear regression analysis was done using the GraphPad Prism program.

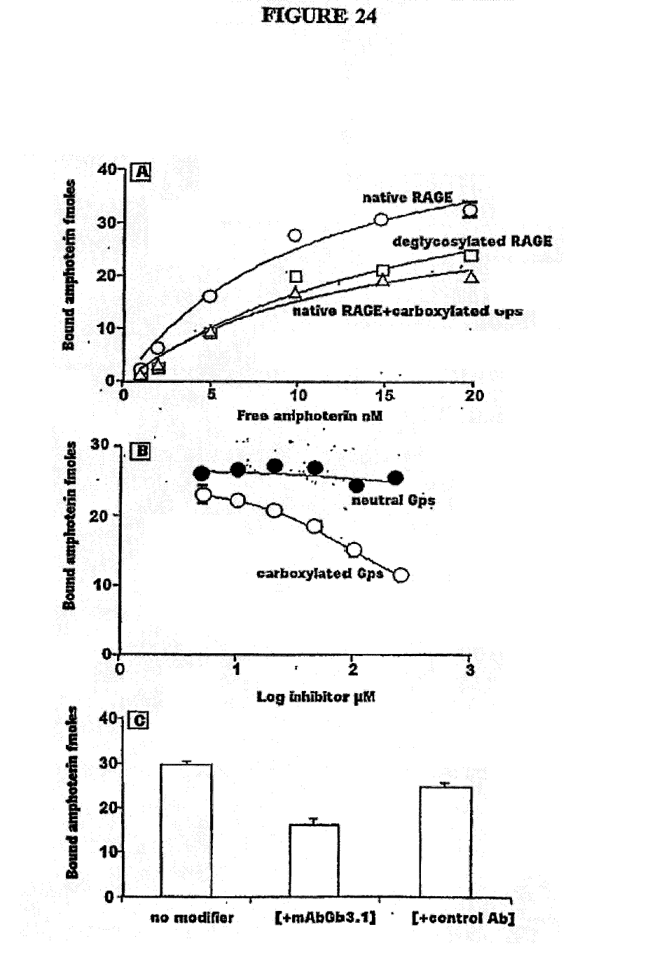

As shown in FIG. 24, Panel A, purified RAGE specifically binds ¹²⁵I amphoterin, with a Kd of approximately 10.7±1.9 nM and a Bmax of approximately 52.7±4.3 fmole/well (binding potential Bmax/Kd of 4.9; See, FIG. 24), consistent with previous observations using recombinant rat amphoterin and bovine lung RAGE (Hori et al., [1995], supra). Specific binding is defined as the total binding minus non-specific binding measured in presence of 100-fold excess of unlabeled amphoterin. The non-specific.15 binding was 3-4% of total binding. Then, the possibility that amphoterin binding is dependent upon the N-glycans on RAGE was examined. Non-linear transformations showed that the binding potential of amphoterin-RAGE (Bmax/Kd) is significantly reduced in presence of soluble COO⁻ glycopeptides (Kd of approximately 12.6±2.7 nM and a Bmax of approximately 34.7±3.7 fmole/well; Bmax/Kd=2.8), while it is unaffected by neutral glycopeptides (data not shown).

In addition, it was possible to effectively deglycosylate RAGE using PNGaseF without prior protein denaturation (data not shown), suggesting that the glycans are exposed and readily accessible in the native protein. Deglycosylation again significantly reduces the binding potential (Kd of approximately 18.2±5.3 nM and a Bmax of approximately 47.9±_{—}7.9 fmole/well; Bmax/Kd=2.6). The binding of a single concentration of labeled ligand was also tested in presence of various concentrations of COO⁻ or neutral glycopeptides. Again, while the COO⁻ glycopeptides progressively inhibit binding, neutral species are without effect (FIG. 24, Panel B). Binding was also found to be reduced in presence of mAbGB3.1, while a non-relevant antibody is ineffective (FIG. 24, Panel C). The inability of COO⁻ glycopeptides, mAbGB3.1 or of RAGE deglycosylation to completely block binding indicates that the interaction between amphoterin and RAGE occurs at more than one site. However, these results strongly imply that glycans on RAGE are important in defining conformational epitopes on amphoterin-binding V-domain of the native receptor.

### Example 33

### Neurite Outgrowth Assays and Immunohistochemistry

Chamber slides were coated with recombinant amphoterin (20 mg/ml) for 16 h at 37° C. Cortical neurons were isolated from the cerebral hemispheres of day 15 mouse embryos as described (Miura et al., J. Neurochem., 76:413-424 [2001]), plated on amphoterin coated slides, and incubated for 24 h in neurobasal medium containing B27 serum substituent (both from GibcoBRL) in the presence and absence of modifiers. Cells were washed, fixed with 4% buffered paraformaldehyde, and stained with lipophilic neuronal tracers DiO or DiI (Molecular Probes).

In separate experiments, cells were also grown on BSA-conjugated COO⁻ or CONHMe glycopeptides (20 mg/ml, generated as described above) coated on polylysine matrices. About 70% of the added conjugates bound to the matrices based on protein estimation before and after coating (data not shown). Morphometric analysis of neurite lengths was made on captured images of fixed, DiI stained cultures. Neurite-bearing cells were defined as cells having neurites greater than one cell body diameter. Neurite outgrowth assays using N18 mouse neuroblastoma cells stably transfected with full length or cytoplasmic domain-deleted RAGE were performed as known in the art (See, Huttunen et al., J. Biol. Chem., 274:19919-19924 [1999]; and Huttunen et al., J. Biol. Chem., 275:40096-40105 [2000]). For immunochemical localization of mAbGB3.1 glycotope, embryonic cortical neurons were grown on amphoterin substrate and fixed as above. They were blocked with 3% BSA in PBS, incubated with mAbGB3.1 or non-specific control IgG, followed by FITC-conjugated secondary antibody and examined by standard immunofluorescence microscopy.

RAGE and amphoterin co-localize at the leading edges of various motile cells, including embryonic neurons and tumor cells. Their interaction is crucial for embryonic neurite outgrowth and tumor cell invasion (Huttunen et al., [1999], supra; Huttunen et al., [2000], supra; Rauvala et al., [2000], supra; and Taguchi et al., [2000], supra). To determine if the carboxylated glycans mediated this interaction in vitro, experiments were conducted in order to establish whether embryonic cortical neurons expressed mAbGB3.1 glycotope.

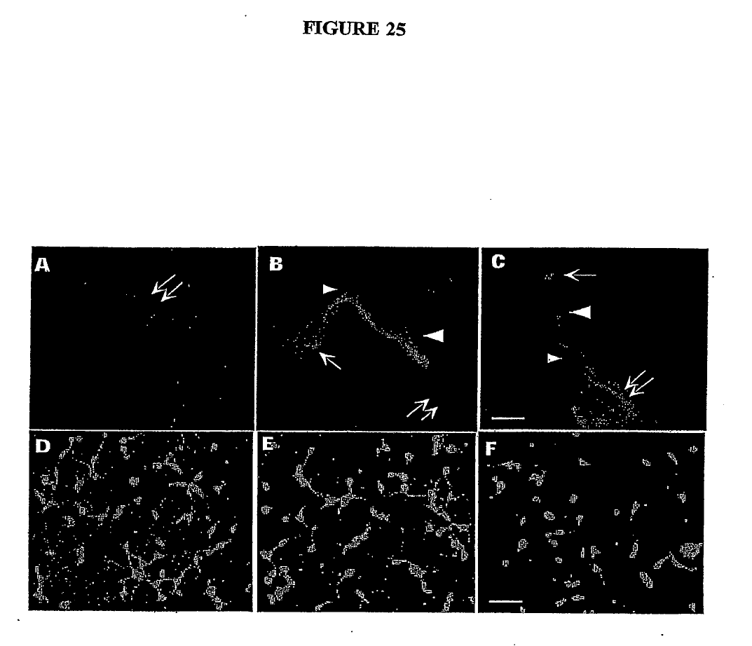

mAbGB3.1 was found to stain axons and growth cones of embryonic cortical neurons grown on amphoterin, as shown in FIG. 25, Panels B and C. Cells incubated with a control IgG are negative, as shown in FIG. 25, Panel A. It was also determined that soluble mAbGB3.1 inhibits neurite outgrowth (See, FIG. 25, Panel F), but a control antibody does not (See, FIG. 25, Panel E). In the absence of a modifier, the % of total cells bearing neurites is 38.7±10.9 (mean±SD) as determined by examination of 4-6 different fields in two replicate analyses. In the presence of mAbGB3.1, the neurites are shorter, and the percentage of cells with neurites is significantly reduced (14.9±6.9, p<0.005). Migrating cells are known to secrete amphoterin (Parkkinen et al., [1993], supra). Cell surface localization of amphoterin has also been previously demonstrated (Rauvala et al., [1988], supra). It was also reasoned that COO⁻ glycopeptides coated on the culture dish could provide an excellent amphoterin-binding surface and thus promote extension of processes. Also, immunoglobulin superfamily members quite often homophilically interact. Though it is not known if RAGE ectodomain is able to bind to itself, it is likely that coated glycopeptides could promote neurite outgrowth by interacting with RAGE itself, or by forming a RAGE-amphoterin-immobilized glycan complex.

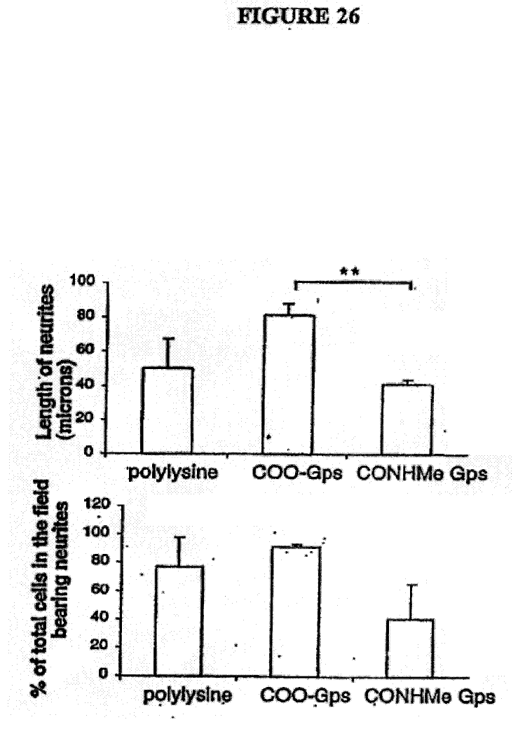

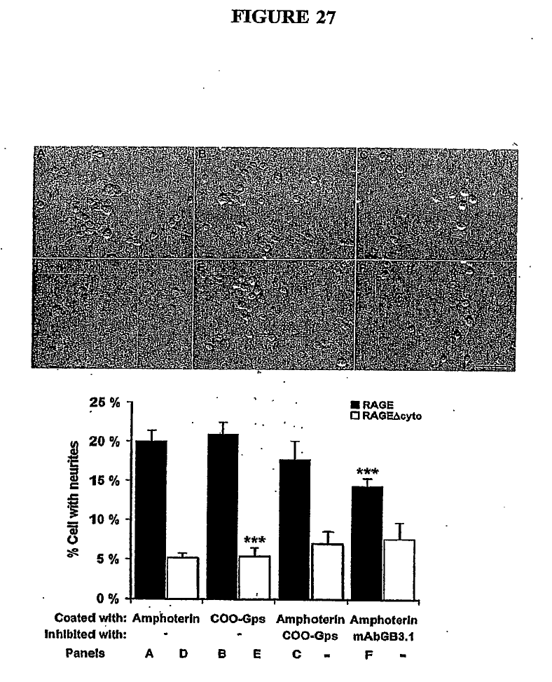

While an understanding of the mechanism(s) is not necessary in order to use the present invention, experiments were conducted to test the growth of cortical neurons on BSA conjugated glycopeptides immobilized on polylysine matrices. It was found that COO⁻ glycopeptides enhance neurite outgrowth, but CONHMe glycopeptides do not, as shown in FIG. 26. Both the number of neurite-bearing cells, and the length of the neurites decrease on CONHMe-glycopeptide substrate. To confirm that the effects of glycopeptides and mAbGB3.1 on neurite outgrowth are RAGE-dependent, they were tested in N18 mouse neuroblastoma cells transfected with either full-length or cytoplasmic domain-deleted RAGE. This domain is crucial for RAGE signaling (Huttunen et al., [1999], supra; and Taguchi et al., [2000], supra). Cells that express the tail-deleted form cannot extend neurites on surfaces coated with RAGE ligands such as amphoterin, whereas the cells expressing the full-length RAGE display a clear neurite morphology (Huttunen et al., [1999], supra; and Huttunen et al., [2000], supra). Parental neuroblastoma cells show very low mAbGB3.1 reactivity (data not shown), while amphoterin is expressed and secreted by N18 cells (Merenmies et al., J. Biol. Chem., 266:16722-16729 [1991]). As shown in FIG. 27, mAbGB3.1 significantly inhibits amphoterin-induced outgrowth of N18 neuroblastoma cell neurites. The neurites are shorter and have a distorted morphology.

Soluble COO⁻ glycopeptides at 200 μM do not block amphoterin-induced neurite outgrowth. However, as indicated above for cortical neurons, when the glycans were used as immobilized matrices, they were fully capable of inducing neurite outgrowth. These neurite outgrowth promoting effects of glycopeptides are RAGE-dependent, since cells expressing the cytoplasmic domain-deleted RAGE do not display neurite morphology. Collectively, the results indicated that amphoterin binds carboxylated N-glycans; RAGE is a glycoprotein containing these glycans; and the glycans play an important role in RAGE-amphoterin binding and signaling in neurite growth.

### Example 34

### Antibody mAbGB3.1 Blocks Acute Peritoneal Inflammation in a Mouse Model of Colitis and Crohn's Disease by Preventing Neutrophil Extravasation

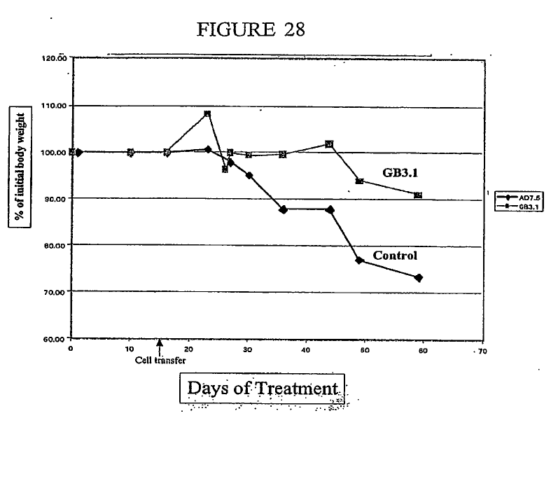

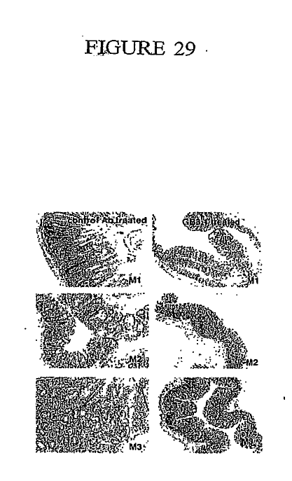

As discussed above, the carboxylated glycans bind to four proteins: annexin-I, S100A8/A9 and S100A12, and amphoterin, which have been variously linked to inflammation and sepsis. The glycans are present on RAGE, which is a signal-transducing receptor for S100 proteins and amphoterin, and is also a key progression factor in the pathology of colitis, arthritis, diabetes and malignancy. mAbGB3.1 blocks inflammation in a Th-1 mediated model of murine colitis. Inflammation was first induced by transfer of CD4+ CD45RBhigh T cells to Rag-1−/− immune deficient mice. Animals were injected with a non-blocking control antibody or mAbGB3.1 and monitored for 6 weeks following cell transfer. Animals given the control antibody showed weight loss and distress, diarrhea, severe colonic inflammation, and 30% of the mice died. This contrasted with mice given mAbGB3.1, which remained healthy, active, showed no significant weight loss, and retained normal colon histology. This is shown in FIGS. 28 and 29. mAbGB3.1 epitopes were strongly expressed in the lamina propria cells of normal mouse intestine and flow cytometric analysis showed co-localization of mAbGB3.1 reactive epitopes on CD4+, CD8+ and CD11b+ cells from intestine and colon of normal mice.

FIG. 28 shows the time-dependent loss of body weight of mice treated with control antibody or with antibody mAbGB3.1. Mice treated with the control lost an average of 25% weight and showed signs of stress, inactivity, and diarrhea. One third of these mice died. Mice treated with mAbGB3.1 appeared completely healthy and did not lose more than 8% body weight during the experiment. This data demonstrates that mAbGB3.1 prevented the onset of colitis symptoms and weight loss in the colitis-sensitive immune-deficient mice.

FIG. 29 shows the histological analysis of the colon from immune-deficient mice treated with a control monoclonal antibody or with mAbGB3.1. Colitis was induced in immunodeficient mice by administering proinflammatory CD4+ CD45RBhi T-cells. Untreated/control antibody treated mice developed diarrhea, weight loss, and moderate to severe inflammation of the colon (colitis). ⅓ of the mice died. mAbGB3.1 treatment blocked onset of colitis. All mice were healthy and survived till the end of the experiment. Those treated with the experimental antibody appeared normal while those treated with the control antibody showed extensive inflammation and abnormal appearance of the tissue. This data demonstrates that mAbGB3.1 prevents colonic inflammation in a mouse model of colitis. This is consistent with the normal appearance of the mice treated with mAbGB3.1. These results also suggest a causative role for the carboxylated glycans in Th-1 mediated inflammatory response in colitis. This data demonstrates that mAbGB3.1 is useful as a therapeutic agent for the treatment of colitis and/or Crohn's disease in humans. Since annexin-I, S100A8/A9 and S100A12, and amphoterin are all involved in inflammation and also bind to the carboxylated glycans recognized by antibody mAbGB3.1, this antibody, or agents that mimic the carboxylated sugar chains are contemplated to be useful to treat, for example, arthritis, diabetes and malignancy.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in diagnostics, cell signaling, and/or related fields are intended to be within the scope of the present invention.

1-52. (canceled)

53. A hybridoma cell line that produces a monoclonal antibody selected from the group consisting of mAbEE4.1, mAbGB3.1, mAbB2.6, and mAbEH2.7.

54. A hybridoma cell line that produces monoclonal antibody mAbGB3.1.

55-122. (canceled)

123. An antibody produced by the hybridoma cell line of claim 53.

124. An antibody produced by the hybridoma cell line of claim 54.
